(19) United States
(12) Patent Application Publication
(10) Pub. No.: US 2024/0082393 A1
(43) Pub. Date: Mar. 14, 2024

(54) ADJUVANTS TO STIMULATE BROAD AND PERSISTENT INNATE IMMUNITY AGAINST DIVERSE ANTIGENS

(71) Applicant: The Board of Trustees of the Leland Stanford Junior University, Stanford, CA (US)

(72) Inventors: Bali Pulendran, Redwood City, CA (US); Florian Wimmers, Palo Alto, CA (US)

(21) Appl. No.: 18/270,996

(22) PCT Filed: Jan. 18, 2022
(86) PCT No.: PCT/US2022/012801

§ 371 Date: Jul. 5, 2023

Related U.S. Application Data
(60) Provisional application No. 63/138,163, filed on Jan. 15, 2021.

(51) Int. Cl. A61K 39/39; A61K 39/12; A61P 31/16; C12N 7/00

(52) U.S. Cl. CPC A61K 39/39; A61K 39/12; A61P 31/16; C12N 7/00; A61K 2039/55505

(57) ABSTRACT

Methods are provided herein for modulating the epigenome of immune cells by administration of an immunostimulatory composition comprising adjuvants, e.g. vaccine adjuvants, to stimulate broad and persistent innate immunity against pathogens unrelated to antigens present in the composition.

### CROSS REFERENCE TO RELATED APPLICATION

The present application claims the benefit of PCT Application No. PCT/US2022/012801, filed Jan. 18, 2022, which claims priority to U.S. Provisional Patent Application No. 63/138,163, filed Jan. 15, 2021, which applications are incorporated herein by reference in their entirety.

### GOVERNMENT SUPPORT RESEARCH

This invention was made with Government support under contract A1090023 awarded by the National Institutes of Health. The Government has certain rights in the invention.

### BACKGROUND

Recent research has highlighted a central role for the epigenome in the regulation of fundamental biological processes. The epigenome can maintain particular chromatin states over prolonged periods of time that span generations of cells, thus enabling the durable storage of gene expression information. In the context of the immune system, epigenomic events have been described during hematopoiesis, generation of immunological memory and exhaustion in T lymphocytes, and the development of B and plasma cells. Recent studies have also revealed that epigenomic changes in monocytes and NK cells imprint a form of immunological memory in the innate immune system.

The concept of epigenetic imprinting on the innate immune system has acquired a particular significance in the context of vaccination. Vaccination with BCG has been shown to induce epigenomic changes in monocytes, and it has been suggested that such changes result in a durable state of innate activation. However, the extent to which such epigenomic imprinting, observed with BCG vaccination, reflects a more general phenomenon with other vaccines is an open question. Furthermore, the critical parameters that determine vaccination induced epigenomic imprinting, such as the type of vaccine or adjuvant used, or the impact of the microbiome, are not known. Importantly, a comprehensive single-cell analysis of the epigenomic landscape during an immune response to a vaccine, or to any stimulus, in humans is lacking.

Recently, researchers have used systems biological approaches to comprehensively analyze the transcriptional, metabolic, proteomic and cellular landscape in response to vaccination in humans, and identified novel correlates and mechanisms of vaccine immunity. Despite these advances, a comprehensive systems biological assessment of the epigenomic landscape during an immune response in humans is missing.

### SUMMARY

Methods are provided herein for modulating the epigenome of immune cells by administration of an immunostimulatory composition comprising adjuvants, e.g. vaccine adjuvants, to stimulate broad and persistent innate immunity against pathogens, e.g. virus, unrelated to antigens present in the composition. Specifically, innate immune cells, e.g. myeloid cells including monocytes, macrophages, dendritic cells, polymorphonuclear cells (PMN), neutrophils, etc. are epigenetically altered in response to adjuvants, thereby increasing their ability to mount a response against pathogens.

In some embodiments the immunostimulatory composition for administration to an individual comprises an adjuvant but lacks additional antigens, e.g. polypeptides, mRNA encoding polypeptides, DNA encoding polypeptides, complex glycosaccharides, small molecules, siRNA and the like. In some embodiments the immunostimulatory composition for administration to an individual comprises adjuvant and a non-pathogen antigen, e.g. polypeptides, mRNA encoding polypeptides, DNA encoding polypeptides, complex glycosaccharides, and the like derived from a non-pathogenic source. In some embodiments the immunostimulatory composition for administration to an individual comprises adjuvant and antigenic material for influenza virus, e.g. polypeptides, mRNA encoding polypeptides, DNA encoding polypeptides, and the like derived from an influenza virus, where the dose of antigen may be therapeutic or sub-therapeutic.

In some embodiments the adjuvant in an immunostimulatory composition is a water-in-oil emulsion. In some embodiments the emulsion comprises squalene. In some embodiments the adjuvant is AS03 and/or MF59, or TLR ligands, including without limitation TLR7/8 or TLR3 or TLR4 ligands. In some embodiments, the immunostimulatory composition comprises viral vectors. In some embodiments, administration is prophylactic for a viral infection, including without limitation epidemic and pandemic rates of infection. Administration may be repeated at suitable intervals as the immune responsive state fades.

In some embodiments an immunostimulatory composition is administered prophylactically, prior to a period of time in which an individual will be at increased risk of pathogen exposure, including without limitation hospital admission, incarceration, travel, communal living, etc. The pathogen may be a virus, e.g. a respiratory virus. It is shown herein that adjuvants can act as broad immune enhancing agents that engender a broad state of enhanced immune responsiveness for a period of at least about 2 weeks, at least to about 3 weeks, at least to about 4 weeks, and in some instances can be detected after about 2 months, or more. Prophylactic administration may be performed to provide for these periods of increased immune responsiveness during a period of increased risk of pathogen exposure.

The capacity of an individual to respond to pathogen challenge is highly correlated with the epigenetic state of myeloid cells, e.g. monocytes, macrophages and dendritic cells. This state is not static, but rather can be profoundly influenced by prior immune responses, including immunostimulatory composition administration. In particular classical monocytes and myeloid dendritic cells (mDC) are shown to be altered by the immunostimulatory composition administration.

Individuals selected for treatment with the methods of the disclosure may include those with reduced adaptive immune responses, who particularly benefit from enhanced innate immunity. Such individuals may include without limitation, neonates, elderly, individuals being treated with immunosuppressants, e.g. transplant recipients, autoimmune patients, and the like; cancer patients, e.g. those treated with chemotherapeutic drugs or radiotherapy; and the like. For example, a reduced ability to produce antibodies, or other adaptive immune responses, in response to vaccination or exposure can be an indicator of reduced adaptive immune response.

Epigenetic changes that enhance innate immunity can be manifested in enhanced resistance to viral infections, characterized by increased chromatin accessibility at interferon regulatory factor (IRF) loci, enhanced antiviral gene expression, and elevated interferon production. In addition, monocytes and mDC exhibit a state of immune refractoriness (as judged by reduced production of inflammatory cytokines), which state of refractoriness is characterized by reduced histone acetylation and decreased chromatin accessibility at AP-1 loci.

In some embodiments, the effectiveness of administration of an immunostimulatory composition is assessed by analysis of the epigenetic state of immune cells from the individual. Such analysis may be performed on a suitable cell sample, e.g. peripheral blood monocytic cells (PBMC). Cells of particular interest, e.g. CD14+ monocytes and mDC, may be purified, e.g. by selecting for CD14+ cells, for analysis as single cells or in bulk, or may be phenotyped at the single cell level during analysis in the absence of purification. Various methods are known in the art for this purpose, including for example single-cell techniques, including EpiTOF (Epigenetic landscape profiling using cytometry by Time-Of-Flight), single-cell ATAC-seq, and single-cell RNA-seq. Any suitable method for determining histone modification information may be used, e.g. ChIP-Seq, ATAC-seq, etc. EpiTOF panels for mass cytometry may include markers to determine immune cell identity, markers to estimate total histone levels, and markers to assess different histone modifications, including acetylation, methylation, phosphorylation, ubiquitination, citrullination, and crotonylation.

In some embodiments the efficacy of a candidate adjuvant, immunostimulatory composition or administration regimen in enhancing innate immune responsiveness is monitored by detecting the presence of one or more of increased chromatin accessibility at IRF loci, enhanced antiviral gene expression, and elevated interferon production in myeloid cell populations of interest, where increased chromatin accessibility is indicative of continued immune responsiveness.

In other embodiments, a candidate adjuvant is screened for efficacy in enhancing immune responsiveness, by administering the candidate adjuvant to an individual or an animal model, and determining the effect on the epigenetic state of myeloid cells. An adjuvant suitable for the purposes described herein can induce a responsiveness state in relevant myeloid cells, and may be selected for administration.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. Trivalent inactivated seasonal influenza vaccine (TIV) alters the global histone modification profile of immune cells. (A) Study overview. Healthy subjects (n=21) were vaccinated with TIV at day 0. A subgroup of 11 subjects received an additional oral antibiotic regimen, consisting of neomycin, vancomycin, and metronidazole, between days −3 and 1. The global histone modification profile of PBMCs in these subjects was then determined using EpiTOF. (B) UMAP was used to create a dimensionality-reduced representation of the global histone mark profiles of all immune cell subset. (C) UMAP was used to visualize epigenomic similarity at the sample level. (D, E) The effect size of vaccine-induced changes to the global histone modification profile at day 30 after vaccination compared to day 0 before vaccination were calculated. D) Top-10 most significantly increased and reduced histone modifications. E) Heatmap showing histone modification changes in innate immune cells. Only changes with an FDR<=0.2 are shown. (F) Change in histone modification levels relative to day 0 before vaccination for a set of highly reduced histone modifications in C monos and mDCs. Dots and lines indicate average, error bars indicate standard error of mean. (G) Histone modification levels of H2BK5ac, H3K37ac, H3K9ac, H4K5ac, and PADI4 were used to generate a UMAP representation of single monocytes and mDCs at all time points. Left panel: cell density at each time point, right panel: H3K27ac levels in each single cell.

FIG. 2. TIV-induced histone modification changes correlate with cytokine production. (A) Schematic overview of experiment. PBMCs from subjects in the EpiTOF experiment were stimulated with three cocktails of synthetic TLR ligands, mimicking bacterial (10 μg/mL Pam3, 25 ng/mL LPS, 300 ng/mL Flagellin) and viral (25 μg/mL pl:C, 4 μg/mL R848) pathogen-associated molecular patterns. After 24 h, Luminex was used to measure the cytokine concentration in supernatants. (B) Heatmap showing the relative change in cytokine concentration at indicated time points compared to day 0. (C) Cytokine concentration at day 0 before and day 30 after vaccination for each investigated subject. Wilcoxon signed rank test was used for hypothesis testing. *p<=0.05, **p<=0.01, ***p<=0.001, ****p<=0.0001. (D) Change in cytokine concentration relative to day 0 for cytokines in C. Dots and lines indicate average. (E, F) Pearson correlation was used to correlate the cytokine concentration of the 10 cytokines in C) with histone modification levels in C monos as well as C mono frequency in PBMCs as determined by EpiTOF and sample viability. E) Boxplots show correlation coefficient for each cytokine after stimulation with either viral or bacterial cocktail. F) Scatter plots for the indicated histone modifications and cytokines. (G, H,) PBMCs from healthy donors were pre-treated with the pharmacological inhibitors A-485 (P300/CBP), and CI-Amidine (PADI4) for 2 h and subsequently stimulated with either LPS (25 ng/mL) or R848 (4 μg/mL) for 6 h. BrefA was added for the last 4 h of stimulation. H3K27ac, total H3, IL-1b and TNFα levels were measured using intracellular flow cytometry. G) Gating scheme showing the production of IL-1b and TNFa in C monos after indicated treatment. H) Boxplot summary of the fraction of IL-1b+ or TNFa+ cells in multiple donors. Wilcoxon rank sum test, *p<=0.05, **p<=0.01, ***p<=0.001, ****p<=0.0001, n=4-11

FIG. 3. TIV induces reduced chromatin accessibility in immune response genes and AP-1 controlled regions. (A) Schematic overview of the experiment. C monos, mDCs, and pDCs were isolated from PBMCs of vaccinated subjects (n=8) at day −21 (BL), 0 and 30 using FACS. ATAC-seq and RNA-seq were used to analyze the chromatin accessibility landscape as well as the transcriptional landscape in these cells. (B) Differentially accessible chromatin regions (DARs) at day 30 after vaccination compared to day 0 before vaccination were identified using DESeq2. Pval<=0.05. (C) Heatmap representation of the normalized accessibility at the top 200 as well as cytokine-associated DARs in C monos for each analyzed sample. p: promoter −2000 bp to +500 bp; d: distal −10 kbp to +10 kbp—promoter; t: trans<−10 kbp or >+10 kbp. (D) Network representation of gene set enrichment analysis of DARs in C monos using the Reactome database. Shown are significantly enriched terms with p<=0.05. Color indicates whether majority of enriched regions showed enhanced (red) or reduced (blue) accessibility. Heatmaps show signed −log 10(pval) for significantly enriched terms in highlighted clusters. (E) Motif-based overrepresentation analysis of transcription factor binding sites in DARs at day 30 compared to day 0. (F) Scatter plot showing the change in TF gene expression (x-axis) plotted against the enrichment in DARs for selected transcription factors in the Encode database. Blue color indicates AP-1 members with significantly reduced expression. (G) The change in gene expression of AP-1 family members was calculated using bulk transcriptomics data from 3-9 independent flu vaccine trials previously conducted. Heatmap indicates average log 2 fold change in gene expression over all trials. N indicates subject and study number at each time point. Wilcoxon signed rank test, *p<=0.05, **p<=0.01, ***p<=0.001. (H) DARs in indicated cell type were correlated with H3K27ac levels as measured by EpiTOF and DARs with correlation coefficient>0.5 were analyzed for transcription factor target gene enrichment using the Encode database. Blue color indicates significantly changed AP-1 members. (I, J) PBMCs from healthy donors were pre-treated with the pharmacological inhibitors A-485 (P300/CBP), and CI-Amidine (PAD14) for 2 h and subsequently stimulated with either LPS (25 ng/mL) or R848 (4 μg/mL) for 6 h. BrefA was added for the last 4 h of stimulation. Phospho-c-Jun levels were measured using intracellular flow cytometry. Histogram showing the level of phospho-c-Jun in C monos in the indicated conditions (I). Box plot summary of the fraction of phospho-c-Jun positive cells in C monos (J). Wilcoxon rank sum test, *p<=0.05, **p<=0.01, ***p<=0.001, ****p<=0.0001, n=4-11

FIG. 4. Heterogeneity within monocyte population drives TIV induced epigenomic changes. (A) Schematic overview of the experiment. Innate immune cells were isolated from PBMCs of 3 vaccinated subjects at days 0, 1, and 30, and analyzed using scATAC-seq and scRNA-seq. (B) UMAP representation of scATAC-seq landscape after pre-processing and QC filtering. (C) Heatmap showing the difference in chromatin accessibility at the indicated time points for the top 5 transcription factors per subset. (D) UMAP representation of epigenomic subclusters within the classical monocyte population. (E) Density plot showing the relative contribution of different epigenomic subclusters to the total monocyte population at a given vaccine time point. (F) Variability in TF accessibility within the monocyte population. Value indicates range of accessibility values in all single monocytes. (G) Heatmap showing the difference in chromatin accessibility between monocyte subclusters subset. (H) UMAP representation of monocyte subclusters showing differences in AP-1 accessibility. (I) UMAP representation of monocyte subclusters showing difference in accessibility at Hotspot module 2,3 gene loci. (J) Enrichment analysis of genes associated with loci in Hotspot module 2,3. (K) UMAP representation of the transcriptional landscape of single monocytes. Color indicates expression of genes associated with Hotspot modules 2,3.

FIG. 5. H5N1+AS03 induces repressive epigenomic state akin to TIV. (A) Schematic overview of experiment. Healthy subjects were vaccinated with H5N1 or H5N1 with Adjuvant System 03 (H5N1+AS03) at day 0 and 21. Innate immune cells were isolated from PBMCs at day 0, 21, and 42 and analyzed using scATAC-seq and scRNA-seq (n=2/2). Ex-vivo TLR stimulation and EpiTOF analysis were conducted on PBMCs at days 0, 7, 21, 28, 42. (n=9-13/9-13) (B) UMAP representation of EpiTOF landscape. (C) Histone modification levels in classical monocytes at day 0 and day 42 as measured by EpiTOF. (D) Cytokine concentration in supernatant of TLR-stimulated PBMCs at day 0 and day 42 after vaccination with H5N1+AS03. (C, D) Wilcox signed rank test; *p<=0.05, **p<=0.01; EpiTOF: n=9/9, Luminex: n=13/13. (E) UMAP representation of scATAC-seq (left) and scRNA-seq (right) landscape after pre-processing and QC filtering. (F) Change in accessibility of detected AP-1 family TFs in classical monocytes. Color indicates whether cells are derived from subjects vaccinated with H5N1 (green) or H5N1+AS03 (orange). (G) Overrepresentation analysis of significantly different DARs in classical monocytes using the Reactome database. Color indicates whether enriched genes were predominantly up- or down-regulated. (H) Volcano plot showing changes in expression of AP-1 TF genes in classical monocytes at D42 compared to DO.

FIG. 6. H5N1+AS03 induces epigenomic state of enhanced antiviral immunity. (A) Heatmap showing the change in chromatin accessibility at day 42 vs day 0 for the top5 transcription factors per subset. Color indicates the difference in accessibility, grey fields indicate non-significant changes (fdr>0.05). (B) Line graph showing the difference in transcription factor (TF) accessibility during the course of the vaccine. (C, D) Volcano plot showing the change in gene expression for IRF/STAT TF genes. (E) MA plot showing the average accessibility and log 2(FC) accessibility for genomic regions containing an IRF1 binding motif. Red color indicates regions with significantly changed accessibility (P<=0.05). (F) Gene set enrichment analysis of significantly changed regions in E) occurring in at least 5% of C monos using the Reactome database. (G) Interferon alpha, gamma and IP10 levels were measured in plasma of vaccinated subjects at the indicated time points. Dots and lines indicate average, ribbons indicate standard error of mean. (H5N1/H5N1+AS03: IFNA, n=5/11; IFNG, n=7/14; IP10, n=16/34) (H) Scatter plot showing the change in chromatin accessibility (x-axis) and the change in gene expression (y-axis) at day 21 vs day 0 for C monos (scATAC P<=0.05 and occurring in at least 5% of cells). Indicated statistics are based on Pearson correlation analysis and Chi-square test. (I) Change in gene expression for selected antiviral and interferon-related BTMs in bulk RNA-seq analysis for subjects vaccinated with H1N1 (green) and H1N1+AS03 (orange) at indicated time points. (H1N1: n=16, H1N1+AS03: n=34. (J) Scatter plot showing the change in chromatin accessibility at day 21 vs day 0 in C monos (x-axis) and the significant change (p<=0.05, log 2(FC)>+/−0.03) in vaccine-induced gene expression at the booster vaccination compared to the prime vaccination (y-axis, Day22day21 vs Day1day0). Chi-square test was used to determine whether both variables were related. (K) Bubble plot showing enrichment results using the Encode TF target gene database. Color indicates the original of the analyzed genes in j).

FIG. 7. H1N1+AS03 induces enhanced resistance to in-vitro infection with heterologous viruses. (A) Schematic overview of the experiment. PBMCs from 10 healthy subjects at day 0, 21 and 42 after vaccination with H5N1+AS03 were infected with Dengue virus or Zika virus at an MOI of 1 and cultured for 0, 24 and 48 hours. After culture, viral copy numbers in cell pellet were determined via qPCR. (B) Boxplot showing viral titers in Dengue-, Zika-, and mock-infected samples. (C) Line graph showing the viral growth curve for Dengue virus (red) and zika virus (blue). Dots and lines indicate average, error bars indicate standard error of mean. n>21 samples (D) Log 2 fold change in viral titers relative to day 0 before vaccination. Wilcoxon signed rank test was used to compare changes within group; **p<=0.01, *p<=0.05, n=8-9. (E) Boxplot showing the concentration of IFNa, IFNg, and IP10 in Dengue-, Zika-, and mock-infected cultures at 24 h after incubation. Wilcoxon rank-sum test was used to compare groups. (F, G) Pearson correlation analysis of the change in viral titers (d0 vs d21) with change in vaccine-induced, in-vivo expression of enhanced antiviral genes at prime (d0 vs d1) and boost (d21 vs d22) (red genes FIG. 6g). F) Boxplot showing correlation coefficient per viral condition. G) Scatter plot showing change in vaccine-induced expression of IRF1 (x-axis) and viral titers (y-axis). B, E) Wilcoxon rank sum test was used to compare groups.

FIG. 8. Cell type abundance and vaccine induced epigenomic changes by EpiTOF, related to FIG. 1. (A) PBMC viability after thawing by vaccination time point. (B) Change in cell type abundance per subject. Wilcoxon signed rank test was used to compare changes at post-vaccine time points with d0 and p-values were corrected using the FDR approach. No comparison passed the threshold of fdr<=0.05. (C) Heatmap showing histone modification changes at day 30 compared to day 0 in all detected immune cell subsets. Changes were calculated using the effect size approach. Only changes with an FDR<=0.2 are shown. (D) Correlation of histone modification changes at day 30 compared to day 0 calculated separately for subjects in the control (x-axis) and antibiotics group (y-axis). For monocytes and mDCs, only significantly changed histone modifications are shown (FDR<=0.2). (E) Correlation matrix showing the pair-wise correlation coefficient between all histone modification in classical monocytes.

FIG. 9. Analysis of vaccine-induced change in gene expression of histone modifying enzyme by bulk transcriptomics, related to FIG. 1. (A) Heatmap showing the log 2 fold change in gene expression relative to day 0 before vaccination. T-test was used for statistical testing. *p<=0.05

FIG. 10. Histone modification profile distance of CD34⁺ progenitor cells by EpiTOF, related to FIG. 1. (A) Cartoon of the analysis approach. The Euclidean distance between the histone modification profile of every single CD34⁺ progenitor cell to an average lymphoid or myeloid profile was calculated. (B) Violin plot showing the histone modification profile distance of single CD34⁺ progenitor cells to a common lymphoid (purple) or myeloid (turquoise) profile at the indicated time point using EpiTOF panel 2. (C) Median change in histone modification profile distance over time. (D) Change in histone modification profile distance of CD34⁺ progenitor cells to indicated cell types at day 30 after vaccination compared to day 0.

FIG. 11. Cytokine production upon TLR stimulation, related to FIG. 2. (A) Dot plot showing log 2 cytokine concentration in each TLR-stimulated PBMC culture by stimulation condition. (B) Heatmap showing the change in cytokine concentration relative to day 0 separately for antibiotics and control subjects.

FIG. 12. Vaccine-induced epigenomic changes by bulk ATAC-seq, related to FIG. 3. (A) DARs at day 30 compared to day 0 (left) and day 0 vs baseline before antibiotics treatment (right, antibiotics subjects only). (B) DARs at day 30 compared to day 0 were calculated separately for control and antibiotics subjects. Log 2 FC values from peaks that were significantly changed in the combined analysis (FIG. 3b) were correlated with each using Pearson.

FIG. 13. Changes in cell abundance and cytokine production upon TLR stimulation, related to FIG. 5. (A) EpiTOF/Luminex PBMC viability after thawing by vaccination time point. (B) Change in cell type abundance per subject as measured by EPITOF. Wilcoxon signed rank test was used to compare changes at post-vaccine time points with d0 and p-values were corrected using the FDR approach. No comparison passed the threshold of fdr<=0.05. (C) Dot plot showing log 2 cytokine concentration in each TLR-stimulated PBMC culture by stimulation condition.

FIG. 14. Model of bi-directional epigenomic reprogramming.

FIG. 15. SARS-CoV-2 RBD-NP immunization induces robust antibody responses. a, Schematic representation of the study design. b, SARS-CoV-2 S-specific IgG titers (plotted as reciprocal EC₅₀) in sera collected at days 21 and 42 measured by ELISA. The box shows median and 25^{th} and 75^{th} percentiles and the error bars show the range. c-d, Serum nAb titers (plotted as reciprocal IC₅₀) determined using a SARS-CoV-2 S pseudovirus (c) and authentic SARS-CoV-2 (d) entry assay at day −7, 21 and 42. In c and d, the black line represents the geometric mean of all data points. The numbers represent geometric mean titers on day 42. Asterisks represent the statistically significant differences between two groups analyzed by two-sided Mann-Whitney rank-sum test (*p<0.05, **p<0.01). e, NAb titers against the authentic SARS-CoV-2 virus measured at time points indicated on X-axis. The numbers represent GMT. Statistical difference between the time points was analyzed by two-sided Wilcoxon matched-pairs signed-rank.

FIG. 16. Adjuvanted RBD-NP immunization elicits nAb responses against emerging SARS-CoV-2 variants. a, Serum nAb titers against the wild-type (circles) or the B.1.1.7 or B.1.351 (squares) variant live-viruses measured in serum collected at day 42, 3 weeks following secondary immunization. The arrows and numbers in brackets within the plots indicate the direction of change in the magnitude of nAb titers against the variant strains and the fold change, respectively. b, The fold change between nAb titers measured against the WT (Wuhan) and the SA (B.1.351) strains in animals from groups indicated on X-axis. The statistical difference between two groups was determined by two-sided Mann-Whitney rank-sum test. c, Serum nAb titers against the wild-type (circles) or the B.1.351 (squares) variant live-viruses measured on day 42 or day 154. The statistical difference between the time points was determined by two-sided Wilcoxon matched-pairs signed-rank. The numbers within the plots indicate GMT.

FIG. 17. Cell-mediated immune responses to SARS-CoV-2 RBD-NP immunization. a-b, RBD-specific CD4 T cell responses measured in blood at time points indicated on the x axis. CD4 T cells secreting IL-2, IFN-γ, or TNF-α were plotted as Th1-type responses (a) and the Th2-type responses show the frequency of IL-4-producing CD4 T cells (b). c, Pie charts representing the proportions of RBD-specific CD4 T cells expressing one, two, or three cytokines as shown in the legend. d, Flow cytometry plots showing expression of IL-21 and CD154 after ex vivo stimulation with DMSO (no peptide, top) or an overlapping peptide pool spanning the SARS-CoV-2 RBD (bottom). e, RBD-specific CD154⁺±IL-21⁺CD4⁺ T cell responses measured in blood at day 28. Asterisks represent statistically significant differences. The differences between groups were analyzed by two-sided Mann-Whitney rank-sum test and the differences between time points within a group were analyzed by two-sided Wilcoxon matched-pairs signed-rank test (*p<0.05, ** p<0.01).

FIG. 18. Protection against SARS-CoV-2 challenge. a-b, SARS-CoV-2 viral load in pharynges (a) and nares (b) of vaccinated and control macaques measured using subgenomic E gene PCR. c, Peak (day 2) viral load in pharyngeal and nasal compartments in each group. d, Viral load in BAL fluid measured using subgenomic N gene PCR. e, Inflammation in the lungs of two animals from each group indicated in the legend, pre-challenge (day 0) and post-challenge (day 4 or 5 after infection), measured using PET-CT scans. f, Representative PET-CT images of lungs from one animal in each group. In a, b, and d, the numbers within each box denote the number of infected animals per total number of animals in each group. PET signal is scaled 0 to 15 SUV. Statistical differences between groups were measured using two-sided Mann-Whitney rank-sum tests (*p<0.05, **p<0.01).

FIG. 19. Immune correlates of protection. a, Heatmap showing Spearman's correlation between peak nasal viral load (day 2) and various immune analyses readouts. All measurements were from peak time points (day 42 for antibodies, day 25 for plasmablast, and day 28 for T cell responses). The p-values were calculated for Spearman's correlation and corrected for multiple-testing. Asterisks represent statistical significance. b, Spearman's correlation plots between peak nasal viral load and the top three immune parameters shown in a.

FIG. 20. Functional antibody profiling by systems serology. a-c, SARS-CoV-2 Spike-specific binding IgM (a), IgG1 (b) and IgA (b) responses in sera collected at days 21 and 42. The box shows median and 25^{th} and 75^{th} percentiles and the error bars show the range. d-e, FcR-binding antibody responses, FcR2A-2 (d) and FcR3A (e) measured in serum collected at days 21 and 42. (f) PLSDA analysis of all antibody features measured using systems serology. (g) The top 3 antibody features discriminating protected vs. infected animals on day 42 in the PLSDA analysis. (h) Heatmap showing spearman's correlation between peak nasal viral load (left) or pharyngeal vial load (right) and antibody responses (day 42) indicated on the Y-axis. The p-values were calculated for Spearman's correlation and corrected for multiple-testing. In a-e, the statistically significant difference between two groups were determined by two-sided Mann-Whitney rank-sum test (*p<0.05, **p<0.01, ***p<0.001 and ****p<0.0001).

FIG. 21. RBD-NP or HexaPro immunization with AS03 elicits comparable nAb responses. a, Schematic representation of the study design. b-c, Serum nAb titers (plotted as reciprocal IC₅₀) determined using a SARS-CoV-2 S pseudovirus (b) or authentic SARS-CoV-2 (c) assay at day 21 and 42. The box shows median and 25^{th} and 75^{th} percentiles and the error bars show the range. Asterisks represent statistically significant differences between two groups analyzed by two-sided Mann-Whitney rank-sum test (*p<0.05). Open circles denote animals from the earlier study shown in FIG. 1. d, Neutralizing antibody titers measured against live WT (circle) or B.1.1.7. or B.1.351 variants (squares) in sera collected on day 42 from animals that received soluble HexaPro.

FIG. 22. Structural, biophysical, and antigenic characterization of RBD-16GS-I53-50. a, Structural model of the RBD-16GS-I53-50 (RBD-NP) immunogen. The genetic linker connecting the RBD antigen to the I53-50A trimer is expected to be flexible and thus the RBD may adopt alternate orientations to that shown. b, Negative stain electron microscopy of RBD-NP. Scale bar, 100 nm. c, Dynamic light scattering (DLS) of RBD-NP and unmodified 153-50 lacking displayed antigen. The data indicate the presence of monodisperse nanoparticles with size distributions centered around 36 nm for RBD-NP and 30 nm for 153-50. In b and c, the samples were analyzed following a single freeze/thaw cycle. d, Antigenic characterization by biolayer interferometry (BLI). RBD-NP was bound to immobilized CR3022 mAb and maACE2-Fc receptor, both before and after one freeze/thaw cycle. Monomeric SARS-CoV-2 RBD was used as a reference antigen.

FIG. 23. Comparison of anti-SARS-CoV-2 spike vs. anti-153-50 nanoparticle scaffold antibody responses. a, Serum concentrations of anti-Spike IgG and anti-153-50 nanoparticle IgG (anti-153-50) in individual NHPs detected by ELISA at day 42. Boxes show median and 25^{th} and 75^{th} percentiles and the error bars show the range. The statistical difference between anti-Spike and anti-153-50 IgG response was determined using two-sided Wilcoxon matched-pairs signed-rank test (*p<0.05). b, Spearman's correlation between anti-Spike IgG (described in FIG. 1) and anti-153-50 IgG responses at d-y 42. c-d, Serum nAb titers (plotted as reciprocal IC₅₀) determined using a SARS-CoV-2 S pselvirus (c) and authentic SARS-CoV-2 (d) entry assay at day −7, 21 and 42. In c and d, 5 animals were randomly selected from the AS03 group using “sample” function in R. The black line represents the geometric mean of all data points. The numbers represent geometric mean titers. Asterisks represent the statistically significant differences between two groups analyzed by two-sided Mann-Whitney rank-sum test (*p<0.05, **p<0.01).

FIG. 24. Humoral immune responses. a, Pseudovirus nAb response against human convalescent sera from 4 COVID-19 patients. b, Spearman's correlation between pseudovirus and authentic virus nAb titers measured at day 42. c, RBD-NP-specific IgG secreting plasmablast response measured at day 4 post-secondary vaccination using ELISPOT. The difference between groups was analyzed using two-sided Mann-Whitney rank-sum test (**p<0.01). d, Spearman's correlation between plasmablast response on day 25 and pseudovirus nAb titer measured at day 42.

FIG. 25. Durability and cross-neutralization. a, Pseudovirus nAb response measured in the AS03 durability group at time points indicated in X-axis. b, ACE-2 blocking measured in sera collected at time points indicated on the X-axis. c, SARS-CoV-2 nAb titers against pseudovirus wild-type containing D641G mutation on the Wuhan-1 Spike (circles) or the B.1.1.7 variant (squares) strain measured in day 42 sera.

FIG. 26. Cell-mediated immune responses to RBD-NP immunization. a-b, RBD- and NP-specific CD4 T cell responses measured in blood at time points indicated on the x axis. c, Pie charts representing the proportions of NP-specific CD4 T cells expressing one, two, or three cytokines as shown in the legend. d, Ratio of frequencies of RBD-specific to NP-specific CD4 T cells expressing cytokines indicated within each box. Asterisks represent statistically significant differences. The differences between time points within a group were analyzed by two-sided Wilcoxon matched-pairs signed-rank test (*p<0.05, **p<0.01).

FIG. 27. Clinical parameters before and after SARS-CoV-2 challenge. Clinical parameters measured on the day of challenge, 2 days, 1-, 2- and 3-weeks post SARS-CoV-2 challenge. Body weight (kg), body temperature (° F.), Oxygen saturation (SpO₂) and respiratory rate (BPM) are shown in first, second, third and fourth rows, respectively.

FIG. 28. Neutralizing antibody response post SARS-CoV-2 challenge. Serum nAb titers (plotted as reciprocal IC₅₀) determined using a SARS-CoV-2 S pseudovirus entry assay on the day of challenge, 1, 2 and 3 weeks post challenge. The black line represents the geometric mean of all data points. The circle and triangle shape of the points represent animals protected or infected (in any compartment, i.e., nares, pharynges or BAL), respectively.

FIG. 29. Inflammation in the lung. PET-CT images obtained from the lungs of SARS-CoV-2 infected animals from no vaccine, AS03, or CpG-Alum groups pre-challenge (day 0) and post-challenge (day 4 or 5).

FIG. 30. Cytokine analysis in BAL fluid post SARS-CoV-2 challenge. a, Heatmap showing expression of 24 cytokines measured in BAL fluid collected 1 week post SARS-CoV-2 challenge. b, Expression of Eotxin-3 (CCL26), an eosinophil-recruiting chemokine known to be induced by the Th2 cytokine IL-13, and IL-5, a Th2 cytokine in the BAL fluid collected 1 week post challenge shows no significant increase in vaccinated animals compared to no vaccine controls. c, Abundance of cytokines known to be induced by SARS-CoV-2 infection in humans such as IL-8, MCP-4, IL-6 and IFN-γ in BAL collected 1 week post challenge.

FIG. 31. Immune correlates of protection. a, Heatmap showing Spearman's correlation between peak pharyngeal viral load (day 2) and various immune parameters. All measurements were from peak time points (day 42 for antibodies, day 25 for plasmablast, and day 28 for T cell responses). The p-values were calculated for Spearman's correlation and corrected for multiple-testing using the Benjamini-Hochberg method. b, Spearman's correlation plots between peak nasal (left) or pharyngeal (right) viral load and the frequency of NP-specific IL-2⁺INF-α⁺ CD4 T cells measured at day 28, 1 week after secondary immunization. c, Spearman's correlation between the frequency of NP-specific IL-2⁺INF-α⁺ CD4 T cells measured at day 28 and nAb response measured on day 42.

FIG. 32. Antibody correlates of protection. Heatmap showing spearman's correlation between peak nasal viral load (day 2) and antibody responses indicated on the Y-axis in groups of animals immunized with RBD-NP plus O/W (a), AS03 (b), AS37 (c), CpG-Alum (d) I Alum (e). The p-values were calculated for Spearman's correlation and corrected for multiple-testing.

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “adjuvant” refers to a composition that increases the humoral or cellular immune response of an individual. Adjuvants of interest stimulate the immune system, and as shown herein, alter the epigenomics of innate immune cells to increase responsiveness.

The terms “subject,” “individual,” and “patient” are used interchangeably herein to refer to a mammal being assessed for treatment and/or being treated. In some embodiments, the mammal is a human. The terms “subject,” “individual,” and “patient” encompass, without limitation, individuals having a disease. Subjects may be human, but also include other mammals, particularly those mammals useful as laboratory models for human disease, e.g., mice, rats, etc.

The term “sample” with reference to a patient encompasses blood and other liquid samples of biological origin, solid tissue samples such as a biopsy specimen or tissue cultures or cells derived therefrom and the progeny thereof. The term also encompasses samples that have been manipulated in any way after their procurement, such as by treatment with reagents; washed; or enrichment for certain cell populations, such as diseased cells. The definition also includes samples that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like.

The term “diagnosis” is used herein to refer to the identification of a molecular or pathological state, disease or condition in a subject, individual, or patient.

The term “prognosis” is used herein to refer to the prediction of the likelihood of death or disease progression, including recurrence, spread, and drug resistance, in a subject, individual, or patient. The term “prediction” is used herein to refer to the act of foretelling or estimating, based on observation, experience, or scientific reasoning, the likelihood of a subject, individual, or patient experiencing a particular event or clinical outcome. In one example, a physician may attempt to predict the likelihood that a patient will survive, or the severity of an infection.

As used herein, the terms “treatment,” “treating,” and the like, refer to administering an agent, or carrying out a procedure, for the purposes of obtaining an effect on or in a subject, individual, or patient. The effect may be prophylactic in terms of completely or partially preventing a disease, for example infection by a pathogen, or symptom thereof and/or may be therapeutic in terms of effecting a partial or complete cure for a disease and/or symptoms of the disease.

Treating may refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of an agent to prevent or delay, to alleviate, or to arrest or inhibit development of the symptoms or conditions associated with infectious disease or other diseases. The term “therapeutic effect” refers to the reduction, elimination, or prevention of the disease, symptoms of the disease, or side effects of the disease in the subject.

As used herein, a “therapeutically effective amount” refers to that amount of the immunostimulatory composition sufficient to induce an enhanced immune response. A therapeutically effective amount may refer to the amount of immunostimulatory composition sufficient to reduce infection upon pathogen exposure, e.g., to delay or minimize infection. A therapeutically effective amount may also refer to the amount of the therapeutic agent that provides a therapeutic benefit in the treatment or management of a disease. Further, a therapeutically effective amount means the amount of immunostimulatory composition alone, or in combination with other therapies, that provides a therapeutic benefit in the treatment or management of a disease.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

In some embodiments, for example with clinically approved adjuvants, the unit dose is the same or comparable to the clinically approved dose. For example, a dose for prophylactic purposes disclosed herein may be from about 10% to about 500% of a clinically approved dose of an adjuvant for vaccine administration, and may be from about 25% to about 250%, from about 50% to about 150%, and may be substantially similar in dose.

“In combination with”, “combination therapy” and “combination products” refer, in certain embodiments, to the concurrent administration to a patient of the immunostimulatory compositions described herein in combination with additional therapies, e.g. inclusion of antigenic material, and the like. When administered in combination, each component can be administered at the same time or sequentially in any order at different points in time. Thus, each component can be administered separately but sufficiently closely in time so as to provide the desired therapeutic effect.

“Concomitant administration” means administration of one or more components, such as immunostimulatory compositions, known therapeutic agents, etc. at such time that the combination will have a therapeutic effect. Such concomitant administration may involve concurrent (i.e. at the same time), prior, or subsequent administration of components. A person of ordinary skill in the art would have no difficulty determining the appropriate timing, sequence and dosages of administration.

The use of the term “in combination” does not restrict the order in which prophylactic and/or therapeutic agents are administered to a subject. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure. A “separated” compound refers to a compound that is removed from at least 90% of at least one component of a sample from which the compound was obtained. Any compound described herein can be provided as an isolated or separated compound.

“Antibody” refers to an immunoglobulin molecule that can bind to a specific antigen as the result of an immune response to that antigen. Immunoglobulins are serum proteins composed of “light” and “heavy” polypeptide chains having “constant” and “variable” regions and are divided into classes (e.g., IgA, IgD, IgE, IgG, and IgM) based on the composition of the constant regions.

“Antigen” or “immunogen” refers to any substance that stimulates an immune response. The term includes killed, inactivated, attenuated, or modified live bacteria, viruses, or parasites. The term antigen also includes polynucleotides, polypeptides, recombinant proteins, synthetic peptides, protein extract, cells (including bacterial cells), tissues, polysaccharides, or lipids, or fragments thereof, individually or in any combination thereof. The term antigen also includes antibodies, such as anti-idiotype antibodies or fragments thereof, and to synthetic peptide mimotopes that can mimic an antigen or antigenic determinant (epitope).

“Immune response” in a subject refers to the development of an adaptive immune response, e.g. humoral immune response, cellular immune response, or a humoral and a cellular immune response to an antigen. Immune response also refers to an innate immune response. Immune responses may be determined using standard immunoassays and neutralization assays, which are known in the art.

“Innate Immunity”. The term “innate immunity” refers to immune responses that rely primarily on cells of the myeloid system, not B or T lymphocytes, and that do not generate a memory response. Innate immune responses are not specific to a particular pathogen in the way that the adaptive immune responses are, but rather utilize conserved features of pathogens of pathogen associated immunostimulants to initiate responses.

Pathogen-associated molecular patterns (PAMPs) stimulate two types of innate immune responses: inflammatory responses, and phagocytosis by cells such as neutrophils and macrophages. Both of these responses can occur quickly, even if the host has never been previously exposed to a particular pathogen. PAMPs are of various types, including, for example, formylmethionine-containing peptides, peptidoglycan cell walls and flagella of bacteria, as well as lipopolysaccharide (LPS) on Gram-negative bacteria and teichoic acids on Gram-positive bacteria. They also include molecules in the cell walls of fungi such as zymosan, glucan, and chitin. Many parasites also contain unique membrane components that act as immunostimulants, including glycosylphosphatidylinositol. Short sequences in bacterial DNA can also act as immunostimulants, such as CpG motifs.

PAMPs are recognized by several types of dedicated receptors in the host, that are collectively called pattern recognition receptors, including soluble receptors in the blood (complement) and TLR receptors on the cell surface. TLR receptors initiate phagocytosis, and stimulate gene expression for stimulating innate immune responses. Humans have at least ten TLRs, several of which have been shown to play important parts in innate immune recognition of pathogen-associated immunostimulants, including lipopolysaccharide, peptidoglycan, zymosan, bacterial flagella, and CpG DNA. The different human TLRs are activated in response to different ligands.

The recognition of a microbial invader is usually quickly followed by its engulfment by a phagocytic cell, e.g. macrophages and neutrophils. Macrophages and neutrophils display a variety of cell-surface receptors that enable them to recognize and engulf pathogens. These include pattern recognition receptors such as TLRs. In addition, they have cell-surface receptors for the Fc portion of antibodies produced by the adaptive immune system, as well as for the C3b component of complement.

Pathogens frequently elicit an inflammatory response, which is mediated by a variety of signaling molecules. Activation of TLRs results in the production of both lipid signaling molecules such as prostaglandins and protein (or peptide) signaling molecules such as cytokines, all of which contribute to the inflammatory response. Some of the cytokines produced by activated macrophages are chemoattractants (chemokines). Some of these attract neutrophils, others later attract monocytes and dendritic cells. The dendritic cells pick up antigens from the invading pathogens and carry them to nearby lymph nodes, where they present the antigens to lymphocytes.

“Cellular immune response” or “cell mediated immune response” is one mediated by T-lymphocytes or other white blood cells or both, and includes the production of cytokines, chemokines and similar molecules produced by lymphocyte, leukocytes, or both.

“Immunogenic” means evoking an immune or antigenic response. Thus an immunogenic composition would be any composition that induces an immune response.

“Emulsifier” means a substance used to make an emulsion more stable.

“Emulsion” means a composition of two immiscible liquids in which small droplets of one liquid are suspended in a continuous phase of the other liquid.

“Pharmaceutically acceptable” refers to substances, which are within the scope of sound medical judgment, suitable for use in contact with the tissues of subjects without undue toxicity, irritation, allergic response, and the like, commensurate with a reasonable benefit-to-risk ratio, and effective for their intended use.

“Reactogenicity” refers to the side effects elicited in a subject in response to the administration of an adjuvant, an immunogen, a vaccine composition, etc. It can occur at the site of administration, and is usually assessed in terms of the development of a number of symptoms. These symptoms can include inflammation, redness, and abscess. It is also assessed in terms of occurrence, duration, and severity. A “low” reaction would, for example, involve swelling that is only detectable by palpitation and not by the eye, or would be of short duration. A more severe reaction would be, for example, one that is visible to the eye or is of longer duration.

“Immunostimulatory composition” refers to a composition that includes an adjuvant, as defined herein and may optionally further include an antigen, in which case it may be more conventionally referred to as a vaccine. Administration of the composition to a subject results in an increased responsive state of myeloid immune cells. The amount of a composition that is therapeutically effective may vary depending on the presence of antigen, the adjuvant, and the condition of the subject, and can be determined by one skilled in the art. A non-antigenic adjuvant composition does not comprise an antigen for the disease of interest.

### Adjuvant Compositions

In some embodiments an adjuvant composition is provided for use in an immunostimulatory composition. Exemplary adjuvants include, without limitation, oil in water emulsions, and may comprise squalene in the oil phase. For example, AS03 is an adjuvant system composed of α-tocopherol, squalene and polysorbate 80 in an oil-in-water emulsion. MF59 is another immunologic adjuvant that comprises a squalene emulsion. The dose of adjuvant administered may depend on whether an antigen is present, on the antigen with which it is used and the antigen dosage to be applied. It is also dependent on the intended species and the desired formulation. Usually the quantity is within the range conventionally used for adjuvants. For example, adjuvants typically comprises from about 1 μg to about 1000 μg, inclusive, of a 1-mL dose.

Adjuvants of interest include those approved for clinical use, for example:

| Adjuvant | Composition | Vaccines |
|---|---|---|
| Aluminum | One or more of the following: | Anthrax, DT, DTaP (Daptacel), DTaP |
| | amorphous aluminum | (Infanrix), DTaP-IPV (Kinrix), DTaP- |
| | hydroxyphosphate sulfate (AAHS), | IPV (Quadracel), DTaP-HepB-IPV |
| | aluminum hydroxide, aluminum | (Pediarix), DTaP -IPV/Hib (Pentacel), |
| | phosphate, potassium aluminum | Hep A (Havrix), Hep A (Vaqta), Hep B |
| | sulfate (Alum) | (Engerix-B), Hep B (Recombivax), |
| | | HepA/Hep B (Twinrix), HIB |
| | | (PedvaxHIB), HPV (Gardasil 9), |
| | | Japanese encephalitis (Ixiaro), MenB |
| | | (Bexsero, Trumenba), Pneumococcal |
| | | (Prevnar 13), Td (Tenivac), Td (Mass |
| | | Biologics), Tdap (Adacel), Tdap |
| | | (Boostrix) |
| AS04 | Monophosphoryl lipid A (MPL) + | Cervarix |
| | aluminum salt | |
| MF59 | Oil in water emulsion composed of | Fluad |
| | squalene | |
| AS01_{B} | Monophosphoryl lipid A (MPL) and | Shingrix |
| | QS-21, a natural compound | |
| | extracted from the Chilean soapbark | |
| | tree, combined in a liposomal | |
| | formulation | |
| CpG 1018 | Cytosine phosphoguanine (CpG), a | Heplisav-B |
| | synthetic form of DNA that mimics | |
| | bacterial and viral genetic material | |

TLR agonists are also of interest as adjuvants in immunostimulatory compositions. These compounds activate TLRs. Examples of TLR agonists include pathogen-associated molecular patterns (PAMPs) and mimetics thereof. These microbial molecular markers may be composed of proteins, carbohydrates, lipids, nucleic acids and/or combinations thereof, and may be located internally or externally, as known in the art. Examples include, without limitation, lipopolysaccharide (LPS), zymosan, peptidoglycans, flagellin, synthetic TLR2 agonist Pam3cys, Pam3CSK4, MALP-2, triacylated lipoproteins, lipoteichoic acid, peptidoglycans, diacylated lipopeptides, and the like. The TLR2 ligand may include one or more of lipoteichoic acid (LTA), a synthetic tripalmitoylated lipopeptide (PAM 3 CSK4), zymosan, a lipoglycan such as lipoarabinomannan or lipomannan, a peptidoglycan, diacylated lipoprotein MALP-2, synthetic diacylated lipoprotein FSL-1, heat shock protein HSP60, heat shock protein HSP70, heat shock protein HSP96 or high-mobility-group protein 1 (HMG-1).

TLR3, 4, 7/8 and 9 agonists are of particular interest as immunostimulatory agents. Included in the group are, without limitation: 852A: Synthetic imidazoquinoline mimicking viral ssRNA; VTX-2337: Small-molecule selective TLR8 agonist mimicking viral ssRNA; BCG: Bacillus of Calmette—Guerin, Mycobacterium bovis; CpG ODN: CpG oligodeoxynucleotide; Imiquimod: Synthetic imidazoquinoline mimicking viral ssRNA; LPS: Lipopolysaccharide; MPL: Monophosphoryl lipid A; Poly I:C: Polyriboinosinic-polyribocytidylic acid; PolyICLC: Poly 1:C-poly-1-lysine; Resiquimod: Synthetic imidazoquinoline mimicking viral ssRNA.

Imiquimod is a synthetic imidazoquinoline that targets TLR7. A newer imidazoquinoline TLR7 agonist, 852A, administered parenterally as monotherapy has shown modest clinical efficacy with disease stabilization as a monotherapy. Resiquimod is an imidazoquinoline TLR7/8 agonist in humans.

CpG are single-strand oligodeoxynucleotides (ODNs), characterized by motifs containing cytosines and guanines. Based on their immunologic effects, CpG ODNs are divided into three different classes: CpG-A, a potent stimulator of NK cells owing to its IFN-α-producing effect on pDCs; CpG-B, a moderate IFN-α inducer, and enhancer of antigen-specific immune responses (upregulates costimulatory molecules on pDCs and B cells, induces Th1 cytokine production and stimulates antigen presentation by pDCs); and CpG-C, which combines the stimulatory capacity of both CpG-A and CpG-B. CpG 7909 (PF-3512676, a CpG type B and TLR9 agonist) has been evaluated in several tumor types including renal cell carcinoma, glioblastoma, melanoma, cutaneous T-cell lymphoma and non-Hodgkin's lymphoma. Polyriboinosinic-polyribocytidylic acid (Poly I:C) is a synthetic analog of viral dsRNA that stimulates endosomal (TLR3) and/or cytosolic melanoma differentiation-associated gene 5 (MDA5), leading to increased production of type 1 IFNs. Lipid A molecules that target the TLR4 complex include monophosphoryl lipid A (MPL), a derivative of lipid A from Salmonella minnesota.

Adjuvant formulations for use as immunostimulatory compositions can be homogenized or microfluidized. The formulations are subjected to a primary blending process, typically by passage one or more times through one or more homogenizers. Any commercially available homogenizer can be used for this purpose, e.g., Ross emulsifier (Hauppauge, N.Y.), Gaulin homogenizer (Everett, Mass.), or Microfluidics (Newton, Mass.). In one embodiment, the formulations are homogenized for three minutes at 10,000 rpm. Microfluidization can be achieved by use of a commercial mirofluidizer, such as model number 110Y available from Microfluidics, (Newton, Mass.); Gaulin Model 30CD (Gaulin, Inc., Everett, Mass.); and Rainnie Minilab Type 8.30H (Miro Atomizer Food and Dairy, Inc., Hudson, Wis.). These microfluidizers operate by forcing fluids through small apertures under high pressure, such that two fluid streams interact at high velocities in an interaction chamber to form compositions with droplets of a submicron size. In one embodiment, the formulations are microfluidized by being passed through a 200 micron limiting dimension chamber at 10,000+/−500 psi.

The routes of administration for the adjuvant compositions include parenteral, oral, oronasal, intranasal, intratracheal, topical, etc. Any suitable device may be used to administer the compositions, including syringes, droppers, needleless injection devices, patches, and the like. The route and device selected for use will depend on the composition of the adjuvant, the antigen, and the subject, and such are well known to the skilled artisan.

The adjuvant compositions can further include one or more immunomodulatory agents such as, e.g., quaternary ammonium compounds (e.g., DDA), and interleukins, interferons, or other cytokines. These materials can be purchased commercially. The amount of an immunomodulator suitable for use in the adjuvant compositions depends upon the nature of the immunomodulator used and the subject. However, they are generally used in an amount of about 1 lag to about 5,000 lag per dose. For a specific example, adjuvant compositions containing DDA can be prepared by simply mixing an antigen solution with a freshly prepared solution of DDA.

The adjuvant compositions can further include one or more polymers such as, for example, DEAE Dextran, polyethylene glycol, and polyacrylic acid and polymethacrylic acid (eg, CARBOPOL®). Such material can be purchased commercially. The amount of polymers suitable for use in the adjuvant compositions depends upon the nature of the polymers used. However, they are generally used in an amount of about 0.0001% volume to volume (v/v) to about 75% v/v. In other embodiments, they are used in an amount of about 0.001% v/v to about 50% v/v, of about 0.005% v/v to about 25% v/v, of about 0.01% v/v to about 10% v/v, of about 0.05% v/v to about 2% v/v, and of about 0.1% v/v to about 0.75% v/v. In another embodiment, they are used in an amount of about 0.02 v/v to about 0.4% v/v. DEAE-dextran can have a molecular size in the range of 50,000 Da to 5,000,000 Da, or it can be in the range of 500,000 Da to 2,000,000 Da. Such material may be purchased commercially or prepared from dextran.

The adjuvant compositions can further include one or more Th2 stimulants such as, for example, Bay R1005 ™ and aluminum. The amount of Th2 stimulants suitable for use in the adjuvant compositions depends upon the nature of the Th2 stimulant used. However, they are generally used in an amount of about 0.01 mg to about 10 mg per dose. In other embodiments, they are used in an amount of about 0.05 mg to about 7.5 mg per dose, of about 0.1 mg to about 5 mg per dose, of about 0.5 mg to about 2.5 mg per dose, and of 1 mg to about 2 mg per dose. A specific example is Bay R1005 ™, a glycolipid with the chemical name “N-(2-deoxy-2-L-leucylamino-β-D-glucopyranosyl)-N-octadecyldodecanamide acetate.” It is an amphiphilic molecule which forms micelles in aqueous solution.

Some examples of bacteria causing disease for which non-specific immune responsiveness may be obtained include, for example, Aceinetobacter calcoaceticus, Acetobacter paseruianus, Actinobacillus pleuropneumoniae, Aeromonas hydrophila, Alicyclobacillus acidocaldarius, Arhaeglobus fulgidus, Bacillus pumilus, Bacillus stearothermophillus, Bacillus subtilis, Bacillus thermocatenulatus, Bordetella bronchiseptica, Burkholderia cepacia, Burkholderia glumae, Campylobacter coli, Campylobacter fetus, Campylobacter jejuni, Campylobacter hyointestinalis, Chlamydia psittaci, Chlamydia trachomatis, Chlamydophila spp., Chromobacterium viscosum, Erysipelothrix rhusiopathieae, Listeria monocytogenes, Ehrlichia canis, Escherichia coli, Haemophilus influenzae, Haemophilus somnus, Helicobacter suis, Lawsonia intracellularis, Legionella pneumophilia, Moraxellsa sp., Mycobactrium bovis, Mycoplasma hyopneumoniae, Mycoplasma mycoides subsp. mycoides LC, Clostridium perfringens, Odoribacter denticanis, Pasteurella (Mannheimia) haemolytica, Pasteurella multocida, Photorhabdus luminescens, Porphyromonas gulae, Porphyromonas gingivalis, Porphyromonas salivosa, Propionibacterium acnes, Proteus vulgaris, Pseudomnas wisconsinensis, Pseudomonas aeruginosa, Pseudomonas fluorescens C9, Pseudomonas fluorescens SIKW1, Pseudomonas fragi, Pseudomonas luteola, Pseudomonas oleovorans, Pseudomonas sp B11-1, Alcaliges eutrophus, Psychrobacter immobilis, Rickettsia prowazekii, Rickettsia rickettsia, Salmonella typhimurium, Salmonella bongori, Salmonella enterica, Salmonella dublin, Salmonella typhimurium, Salmonella choleraseuis, Salmonella newport, Serratia marcescens, Spirlina platensis, Staphlyoccocus aureus, Staphyloccoccus epidermidis, Staphylococcus hyicus, Streptomyces albus, Streptomyces cinnamoneus, Streptococcus suis, Streptomyces exfoliates, Streptomyces scabies, Sulfolobus acidocaldarius, Syechocystis sp., Vibrio cholerae, Borrelia burgdorferi, Treponema denticola, Treponema minutum, Treponema phagedenis, Treponema ref ringens, Treponema vincentii, Treponema palladium, and Leptospira species, such as the known pathogens Leptospira canicola, Leptospira grippotyposa, Leptospira hardjo, Leptospira borgpetersenii hardjo-bovis, Leptospira borgpetersenii hardjo-prajitno, Leptospira interrogans, Leptospira icterohaemorrhagiae, Leptospira pomona, and Leptospira bratislava, and combinations thereof.

Examples of viruses causing disease for which non-specific immune responsiveness may be obtained include, for example, SARS-Cov1, SARS-Cov2, and other coronaviruses, Avian herpesviruses, Bovine herpesviruses, Canine herpesviruses, Equine herpesviruses, Feline viral rhinotracheitis virus, Marek's disease virus, Ovine herpesviruses, Porcine herpesviruses, Pseudorabies virus, Avian paramyxoviruses, Bovine respiratory syncytial virus, Canine distemper virus, Canine parainfluenza virus, canine adenovirus, canine parvovirus, Bovine Parainfluenza virus 3, Ovine parainfluenza 3, Rinderpest virus, Border disease virus, Bovine viral diarrhea virus (BVDV), BVDV Type I, BVDV Type II, Classical swine fever virus, Avian Leukosis virus, Bovine immunodeficiency virus, Bovine leukemia virus, Bovine tuberculosis, Equine infectious anemia virus, Feline immunodeficiency virus, Feline leukemia virus (FeLV), Newcastle Disease virus, Ovine progressive pneumonia virus, Ovine pulmonary adenocarcinoma virus, Canine coronavirus (CCV), pantropic CCV, Canine respiratory coronavirus, Bovine coronavirus, Feline Calicivirus, Feline enteric coronavirus, Feline infectious peritonitis, virus, Porcine epidemic diarrhea virus, Porcine hemagglutinating encephalomyletitis virus, Porcine parvovirus, Porcine Circovirus (PCV) Type I, PCV Type II, Porcine Reproductive and Respiratory Syndrome (PRRS) Virus, Transmissible gastroenteritis virus, Turkey coronavirus, Bovine ephemeral fever virus, Rabies, Rotovirus, Vesicular stomatitis virus, lentivirus, Avian influenza, Rhinoviruses, Equine influenza virus, Swine influenza virus, Canine influenza virus, Feline influenza virus, Human influenza virus, Eastern Equine encephalitis virus (EEE), Venezuelan equine encephalitis virus, West Nile virus, Western equine encephalitis virus, human immunodeficiency virus, human papilloma virus, varicella zoster virus, hepatitis B virus, rhinovirus, and measles virus, and combinations thereof. Examples of parasites causing disease for which non-specific immune responsiveness

may be obtained include, for example, Anaplasma, Fasciola hepatica (liver fluke), Coccidia, Eimeria spp., Neospora caninum, Toxoplasma gondii, Giardia, Dirofilaria (heartworms), Ancylostoma (hookworms), Trypanosoma spp., Leishmania spp., Trichomonas spp., Cryptosporidium parvum, Babesia, Schistosoma, Taenia, Strongyloides, Ascaris, Trichinella, Sarcocystis, Hammondia, and Isopsora, and combinations thereof. Also contemplated are external parasites including, but not limited to, ticks, including Ixodes, Rhipicephalus, Dermacentor, Amblyomma, Boophilus, Hyalomma, and Haemaphysalis species, and combinations thereof.

Oil, when added as a component of an adjuvant, generally provides a long and slow release profile. In the present invention, the oil can be metabolizable or non-metabolizable. The oil can be in the form of an oil-in-water, a water-in-oil, or a water-in-oil-in-water emulsion.

Oils suitable for use in the present invention include alkanes, alkenes, alkynes, and their corresponding acids and alcohols, the ethers and esters thereof, and mixtures thereof. The individual compounds of the oil are light hydrocarbon compounds, i.e., such components have 6 to 30 carbon atoms. The oil can be synthetically prepared or purified from petroleum products. The moiety may have a straight or branched chain structure. It may be fully saturated or have one or more double or triple bonds. Some non-metabolizable oils for use in the present invention include mineral oil, paraffin oil, and cycloparaffins, for example.

The term oil is also intended to include “light mineral oil,” i.e., oil which is similarly obtained by distillation of petrolatum, but which has a slightly lower specific gravity than white mineral oil.

Metabolizable oils include metabolizable, non-toxic oils. The oil can be any vegetable oil, fish oil, animal oil or synthetically prepared oil which can be metabolized by the body of the subject to which the adjuvant will be administered and which is not toxic to the subject. Sources for vegetable oils include nuts, seeds and grains.

Other components of the compositions can include pharmaceutically acceptable excipients, such as carriers, solvents, and diluents, isotonic agents, buffering agents, stabilizers, preservatives, vaso-constrictive agents, antibacterial agents, antifungal agents, and the like. Typical carriers, solvents, and diluents include water, saline, dextrose, ethanol, glycerol, oil, and the like. Representative isotonic agents include sodium chloride, dextrose, mannitol, sorbitol, lactose, and the like. Useful stabilizers include gelatin, albumin, and the like.

Surfactants are used to assist in the stabilization of the emulsion selected to act as the carrier for the adjuvant and antigen. Surfactants suitable for use in the present inventions include natural biologically compatible surfactants and non-natural synthetic surfactants. Biologically compatible surfactants include phospholipid compounds or a mixture of phospholipids. Preferred phospholipids are phosphatidylcholines (lecithin), such as soy or egg lecithin. Lecithin can be obtained as a mixture of phosphatides and triglycerides by water-washing crude vegetable oils, and separating and drying the resulting hydrated gums. A refined product can be obtained by fractionating the mixture for acetone insoluble phospholipids and glycolipids remaining after removal of the triglycerides and vegetable oil by acetone washing. Alternatively, lecithin can be obtained from various commercial sources. Other suitable phospholipids include phosphatidylglycerol, phosphatidylinositol, phosphatidylserine, phosphatidic acid, cardiolipin, and phosphatidylethanolamine. The phospholipids may be isolated from natural sources or conventionally synthesized.

Non-natural, synthetic surfactants suitable for use in the present invention include sorbitan-based non-ionic surfactants, e.g. fatty-acid-substituted sorbitan surfactants, fatty acid esters of polyethoxylated sorbitol (TWEEN™), polyethylene glycol esters of fatty acids from sources such as castor oil; polyethoxylated fatty acid, polyethoxylated isooctylphenol/formaldehyde polymer, polyoxyethylene fatty alcohol ethers (BRIJ™); polyoxyethylene nonphenyl ethers (TRITON™), polyoxyethylene isooctylphenyl ethers (TRITON™ X).

As used herein, “a pharmaceutically-acceptable carrier” includes any and all solvents, dispersion media, coatings, adjuvants, stabilizing agents, diluents, preservatives, antibacterial and antifungal agents, isotonic agents, adsorption delaying agents, and the like. The carrier(s) must be “acceptable” in the sense of being compatible with the other components of the compositions and not deleterious to the subject. Typically, the carriers will be will be sterile and pyrogen-free, and selected based on the mode of administration to be used. It is well known by those skilled in the art that the preferred formulations for the pharmaceutically acceptable carrier which comprise the compositions are those pharmaceutical carriers approved in the applicable regulations promulgated by the United States (US) Department of Agriculture or US Food and Drug Administration, or equivalent government agency in a non-US country. Therefore, the pharmaceutically accepted carrier for commercial production of the compositions is a carrier that is already approved or will be approved by the appropriate government agency in the US or foreign country.

The compositions optionally can include compatible pharmaceutically acceptable (i.e., sterile or non-toxic) liquid, semisolid, or solid diluents that serve as pharmaceutical vehicles, excipients, or media. Diluents can include water, saline, dextrose, ethanol, glycerol, and the like. Isotonic agents can include sodium chloride, dextrose, mannitol, sorbitol, and lactose, among others. Stabilizers include albumin, among others.

The compositions can also contain antibiotics or preservatives, including, for example, gentamicin, merthiolate, or chlorocresol. The various classes of antibiotics or preservatives from which to select are well known to the skilled artisan.

### Methods of Use

In some embodiments an immunostimulatory composition, which may comprise, consist or consist essentially of an adjuvant as described above, is administered to an individual to enhance innate immune responsiveness. The individual may be at risk of exposure to a pathogen, e.g. in a pandemic, or other circumstances.

The individual may be administered an immunostimulatory composition prior to a period of time in which an individual will be at increased risk of pathogen exposure, including without limitation: hospital admission, incarceration, travel, entering a communal living situation, etc. The pathogen of increased risk may be a bacteria, virus, parasite, etc., e.g. a respiratory virus.

It is shown herein that adjuvants can act as broad immune enhancing agents that engender a broad state of enhanced immune responsiveness for a period of at least about 2 weeks, at least to about 3 weeks, at least to about 4 weeks, and in some instances can be detected after about 2 months or more. Prophylactic administration may be performed to provide for increased immune responsiveness during a period of increased risk of pathogen exposure.

Administration may be performed once, twice, three or more times as required. Multiple administrations can be spaced apart by about 2, 3, 4, 5, 6, 7, 8 or more weeks initially, and can be further spaced by 2, 3, 4, 5, 6, or more months for subsequent administrations.

Although it is not required, individuals selected for treatment with the methods of the disclosure may include those with reduced adaptive immune responses, who particularly benefit from enhanced innate immunity. Such individuals may include without limitation, neonates, elderly, individuals being treated with immunosuppressants, e.g. transplant recipients, autoimmune patients, and the like; cancer patients, e.g. those treated with chemotherapeutic drugs or radiotherapy; and the like. For example, a reduced ability to produce antibodies, or other adaptive immune responses, in response to vaccination or exposure can be an indicator of reduced adaptive immune response.

In some embodiments, the effectiveness of administration of an immunostimulatory composition is assessed by analysis of the epigenetic state of immune cells from the individual. Such analysis may be performed on a suitable cell sample, e.g. peripheral blood monocytic cells (PBMC). Cells of particular interest, e.g. CD14+ monocytes and mDC, may be purified, e.g. by selecting for CD14+ cells, for analysis as single cells or in bulk, or may be phenotyped at the single cell level during analysis in the absence of purification. Various methods are known in the art for this purpose, including for example single-cell techniques, including EpiTOF (Epigenetic landscape profiling using cytometry by Time-Of-Flight), single-cell ATAC-seq, and single-cell RNA-seq. Any suitable method for determining histone modification information may be used, e.g. ChIP-Seq, ATAC-seq, etc. EpiTOF panels for mass cytometry may include markers to determine immune cell identity, markers to estimate total histone levels, and markers to assess different histone modifications, including acetylation, methylation, phosphorylation, ubiquitination, citrullination, and crotonylation.

In some embodiments the efficacy of a candidate adjuvant, immunostimulatory composition or administration regimen in enhancing innate immune responsiveness is monitored by detecting the presence of one or more of increased chromatin accessibility at IRF loci, enhanced antiviral gene expression, and elevated interferon production in myeloid cell populations of interest, where increased chromatin accessibility is indicative of continued immune responsiveness.

### Screening Methods

In other embodiments, a candidate adjuvant is screened for efficacy in enhancing immune responsiveness, by administering the candidate adjuvant to an individual or an animal model, and determining the effect on the epigenetic state of myeloid cells. An adjuvant suitable for the purposes described herein can induce a responsiveness state in relevant myeloid cells, and may be selected for administration.

Candidate agents of interest are biologically active agents that encompass numerous chemical classes, primarily organic molecules, which may include organometallic molecules, inorganic molecules, etc., including TLR agonists, squalene emulsions, and the like. Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents are screened for biological activity by adding the agent to at least one and usually a plurality of cells, e.g. myeloid cells, or administered to a test animal, usually in conjunction with assay combinations lacking the agent. The change in epigenetics of myeloid cells readout in response to the agent is measured, desirably normalized. In cultures, the agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Epigenetic changes that enhance innate immunity can be manifested in enhanced resistance to viral infections, characterized by increased chromatin accessibility at interferon regulatory factor (IRF) loci, enhanced antiviral gene expression, and elevated interferon production. In addition, monocytes and mDC exhibit a state of immune refractoriness (as judged by reduced production of inflammatory cytokines), which state of refractoriness is characterized by reduced histone acetylation and decreased chromatin accessibility at AP-1 loci.

Kits may be provided. Kits may further include cells or reagents suitable for isolating and culturing cells in preparation for conversion; reagents suitable for culturing T cells; and reagents useful for determining the epigenomic effect of a vaccine adjuvant. Kits may also include tubes, buffers, etc., and instructions for use.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Single-Cell Analysis of the Epigenomic and Transcriptional Landscape of Innate Immunity to Seasonal and Adjuvanted Pandemic Influenza Vaccination in Humans

Emerging evidence indicates a fundamental role for the epigenome in immunity. Here, we used a systems biological approach to map the epigenomic and transcriptional landscape of immunity to influenza vaccination in humans at the single-cell level. Vaccination against seasonal influenza resulted in persistently reduced expression of H3K27ac in monocytes and myeloid dendritic cells, that was associated with impaired cytokine responses to TLR stimulation. Single cell ATAC-seq analysis of 120,305 single cells revealed an epigenomically distinct subcluster of monocytes with reduced chromatin accessibility at AP-1 targeted loci after vaccination. Similar effects were also observed in response to vaccination with the AS03-adjuvanted H5N1 pandemic influenza vaccine. However, this vaccine also stimulated persistently increased chromatin accessibility at loci targeted by the interferon response factors (IRFs). This was associated with elevated expression of antiviral genes and type 1 IFN production, and heightened resistance to infection with the heterologous viruses Zika and dengue. These results demonstrate that influenza vaccines stimulate persistent epigenomic remodeling of the innate immune system. Notably, AS03-adjuvanted vaccination remodeled the epigenome of myeloid cells to confer heightened resistance against heterologous viruses, revealing its unappreciated role as an “epigenetic adjuvant.”

In the current study, we used single-cell techniques, including EpiTOF (Epigenetic landscape profiling using cytometry by Time-Of-Flight), single-cell ATAC-seq, and single-cell RNA-seq, to study the epigenomic and transcriptional landscape of immunity to influenza vaccination in humans. We find that vaccination with the trivalent inactivated seasonal influenza vaccine (TIV) induced global changes to the chromatin state in multiple immune cell subsets, which persisted for up to six months after vaccination. These changes were most pronounced in myeloid cells, which demonstrated a transition to inaccessible chromatin in loci targeted by AP-1 transcription factors, and reduced cytokine production in response to TLR stimulation. Single-cell analysis revealed multiple epigenomic substrates within the monocyte population which drove the observed changes by altering their relative abundance in response to vaccination. Vaccination with the AS03 adjuvanted H5N1 pandemic influenza vaccine also induced similar epigenomic and functional changes in the innate immune system. Strikingly however, AS03 adjuvanted vaccine also induced a concomitantly enhanced state of antiviral vigilance characterized by increased chromatin accessibility at IRF and STAT loci, and heightened resistance against heterologous viral infection.

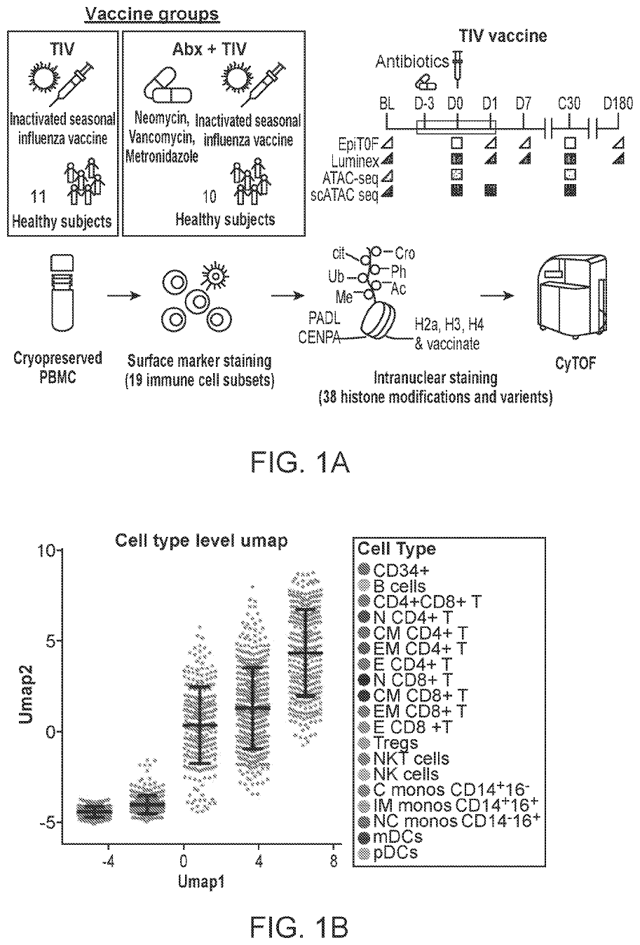

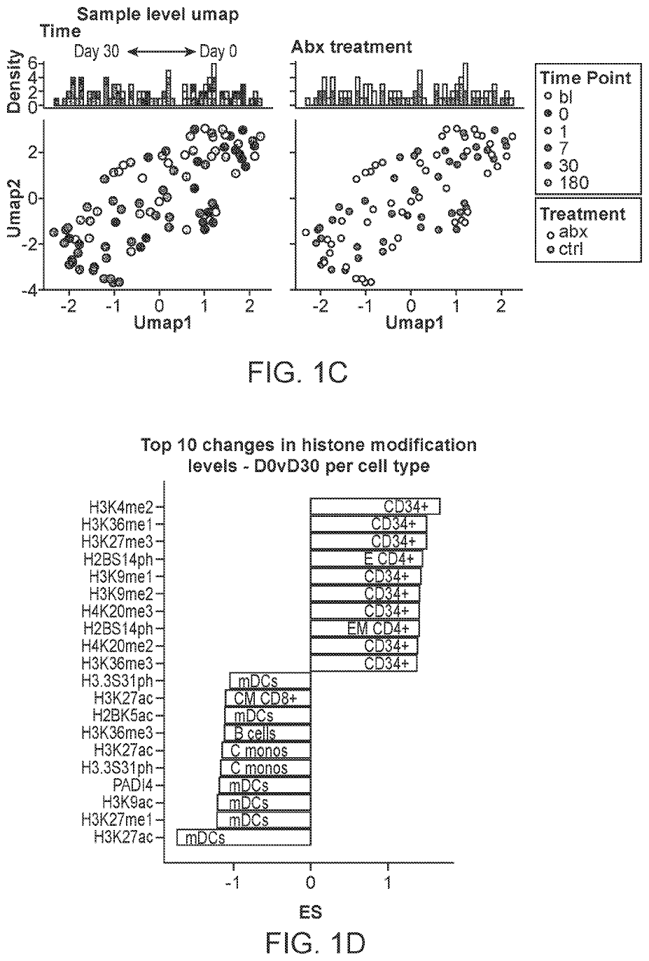

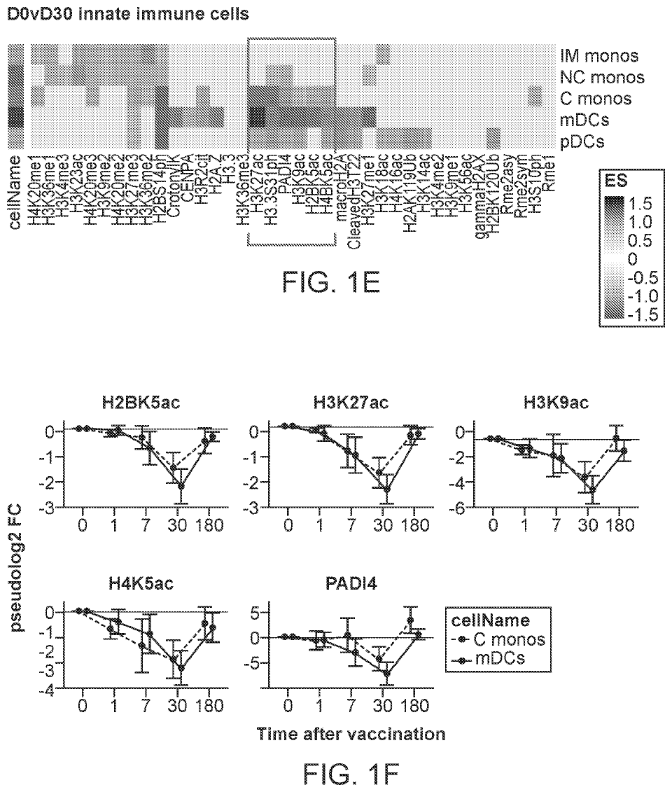

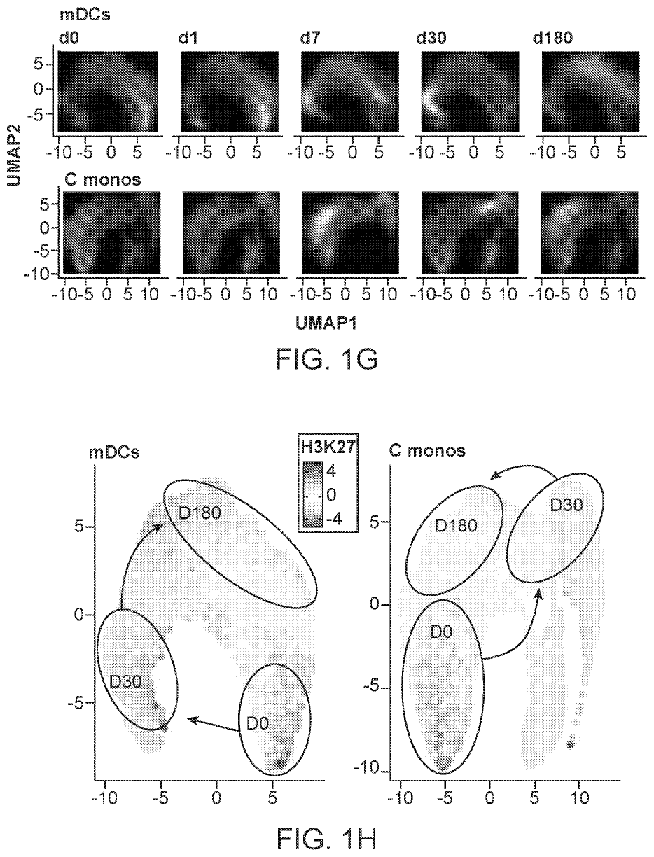

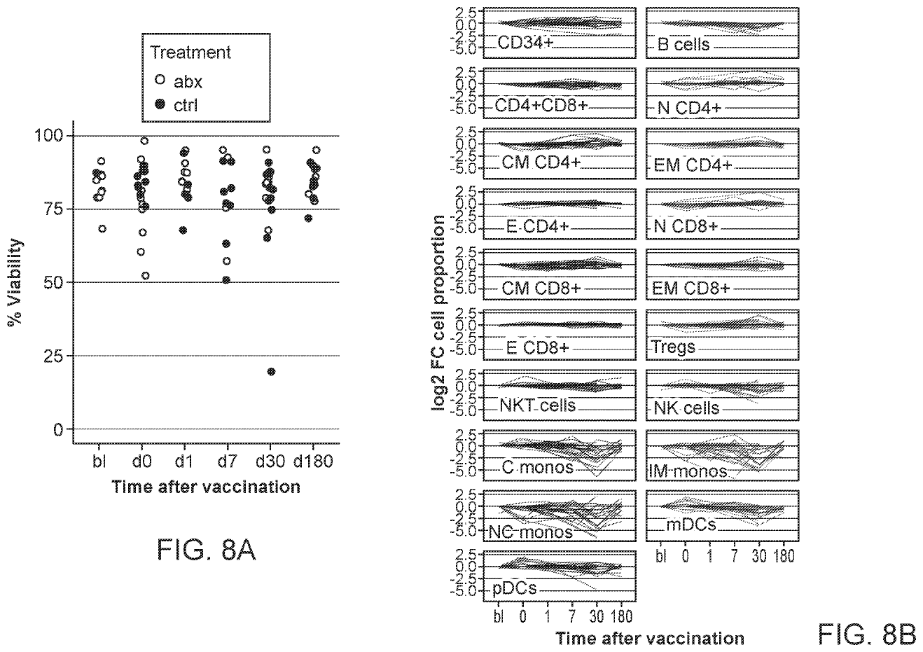

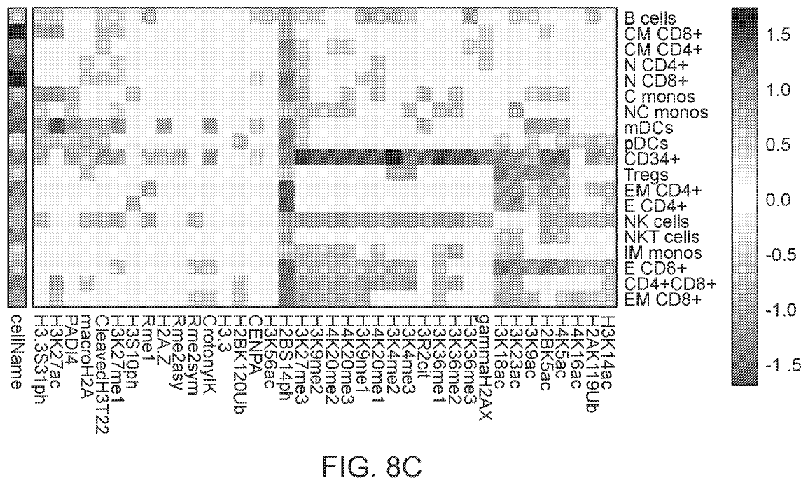

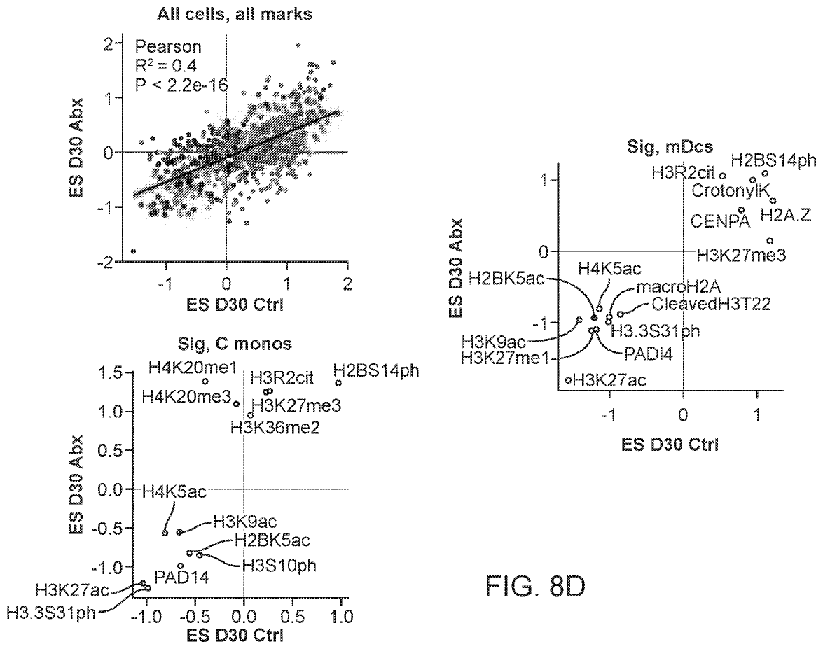

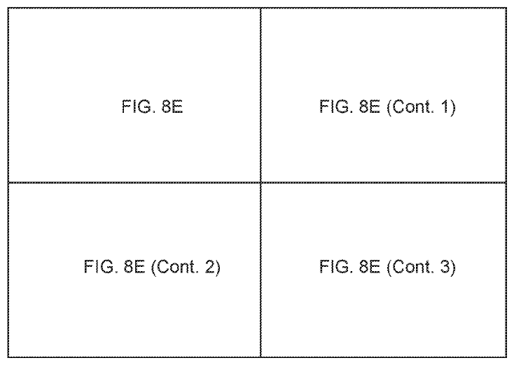

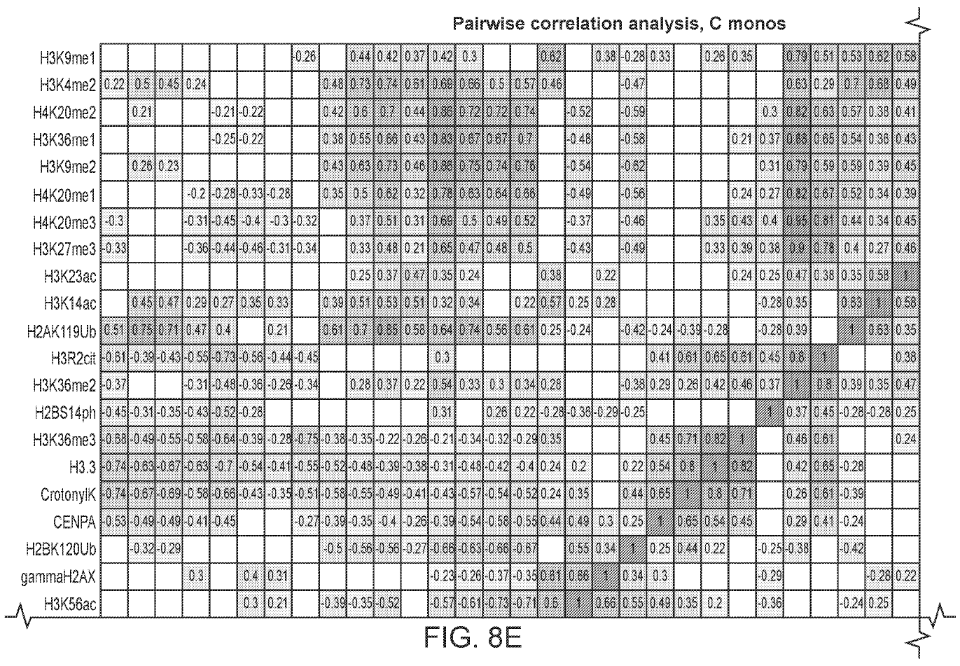

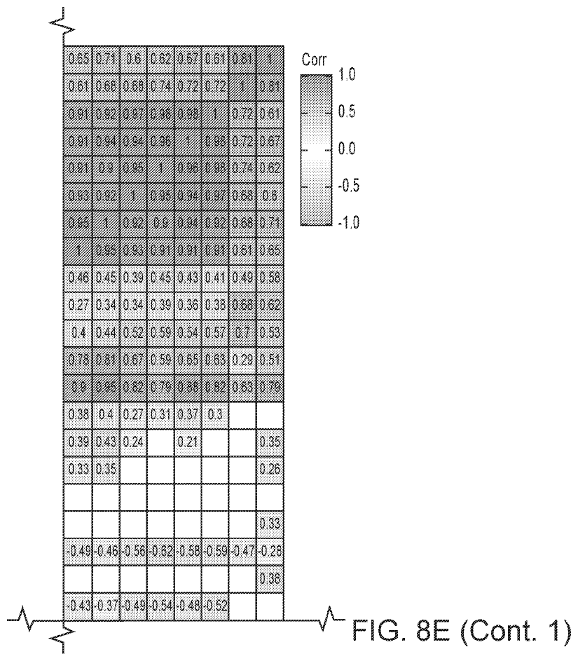

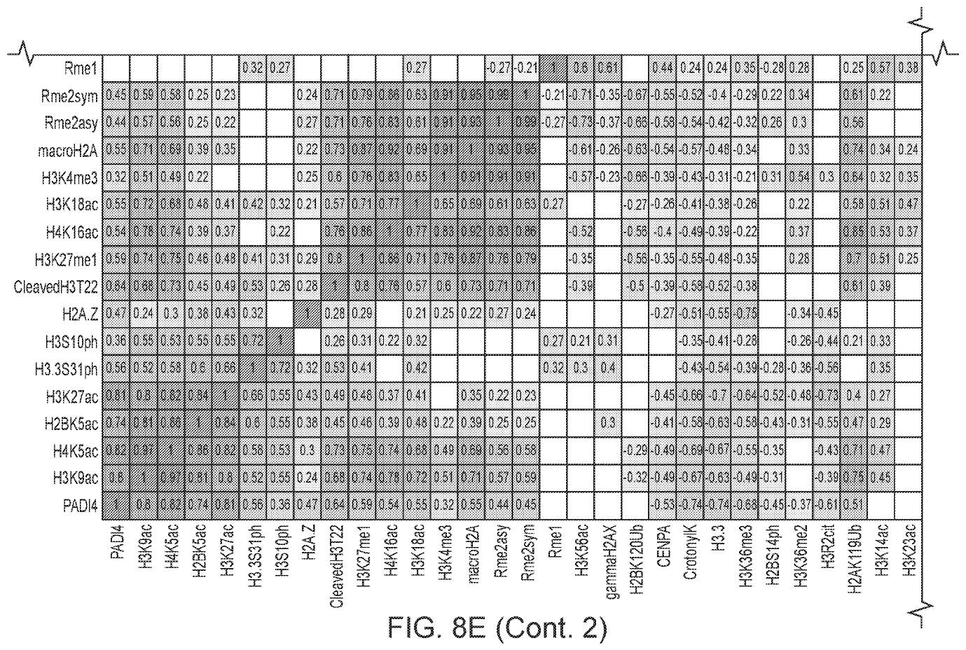

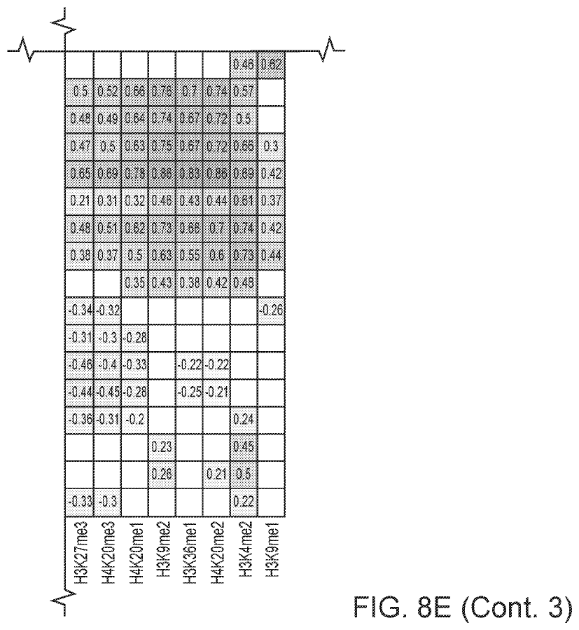

Global epigenomic reprogramming of immune cell subsets after vaccination with TIV. To determine how TIV reprograms the epigenomic landscape of the immune system at the single-cell level, we applied EpiTOF technology to a cohort of 21 healthy individuals aged 18-45. All subjects received TIV at day 0. In order to determine the impact of the gut microbiota on the epigenomic immune cell landscape, a subgroup of 11 subjects received an additional oral antibiotic regimen, consisting of neomycin, vancomycin, and metronidazole, between days −3 and 1 (FIG. 1a). Our previous work with this cohort had demonstrated that antibiotics administration induced significant changes in the transcriptional and metabolic profiles of peripheral blood mononuclear cells (PBMCs). Therefore, we hypothesized antibiotics administration would induce epigenomic reprogramming of PBMCs. To test this hypothesis, we developed two EpiTOF panels of metal-labeled antibodies consisting of 19 markers to determine immune cell identity, 2 markers to estimate total histone levels, and 38 markers to assess different histone modifications, including acetylation, methylation, phosphorylation, ubiquitination, citrullination, and crotonylation. Using these panels, we analyzed PBMCs (FIG. 8a) isolated at day −21 and 0 prior to vaccination, and days 1, 7, 30, and 180 after vaccination. Using a manual gating approach, we detected all major immune cell populations. While the frequency of most immune cell populations did not change during the course of vaccination, we observed a transient increase of pDCs during antibiotics treatment (d0, d1) and a trend towards reduced fractions of myeloid cells in some subjects at later time points (FIG. 8b). Next, we extracted and normalized the histone modification information for each subset and then used this information to generate a UMAP representation of the epigenomic immune cell landscape (FIG. 1b). Histone modification information alone was sufficient to separate the immune cell subsets, and clusters containing lymphoid cells (NK, B, T cells), myeloid cells (monocytes, dendritic cells) and hematopoietic progenitors (CD34⁺) emerged.

Combining the global histone modification data from all immune cell subsets, we observed that samples drawn after vaccination, especially day 30, separated from samples drawn before vaccination indicating that TIV induces changes to the epigenomic immune cell landscape (FIG. 1c). Surprisingly, antibiotics status had no measurable impact on histone modification levels and samples from antibiotics- and control-subjects were intermixed (FIG. 1c). This observation was surprising given the pronounced impact of antibiotics treatment on blood transcriptome and metabolome previously observed in these subjects. Rather we observed changes in the acetylation, methylation, and phosphorylation states of several histone marks in response to vaccination, regardless of the antibiotics status (FIG. 8c, d). In particular, we detected an increase in several histone methylation marks in CD34⁺ cells and a decrease in multiple acetylation and phosphorylation marks in myeloid cells (FIG. 1d). In addition, we observed an increase in H2BS14ph levels in all immune cell subsets at day 30 after vaccination (FIG. 8c). H2BS14ph, together with gammaH2AX, another histone modification, is catalyzed by the protein kinase Mst1 (STK4) and both proteins previously have been associated with apoptosis. However, in our data, we did not observe reduced viability of PBMCs nor increased levels of gammaH2AX at any time point (FIG. 8a). Together, this suggest that, in our case, an apoptosis-induced increase in H2BS14ph is unlikely. Instead, the observed increase in H2BS14ph might be part of the vaccine response as Mst1/STK4 has been shown to be involved in modulation of immune cell activity.

Persistent epigenomic reprogramming in myeloid cells. Interestingly, we observed the strongest changes in histone modification levels in a group of histone acetylation marks, including H2BK5ac, H3K9ac, and H3K27ac which were all reduced in dendritic cells and classical monocytes at day 30 (FIG. 1d, e). Pairwise correlation analysis demonstrated high correlation coefficients between these marks and two additional histone modifications, H4K5ac and PADI4 (Figure S1e). Longitudinal analysis of these five histone modifications demonstrated similar kinetics with a reduction that starts around day 1 to 7 after vaccination, reaches a nadir at day 30, and almost returns to baseline levels at day 180 (FIG. 1f), indicating an epigenomic reprogramming in myeloid cells that persisted for well over a month after vaccination. Non-classical monocytes showed no change in these marks. Importantly, these changes were observed, both in antibiotics- and control-subjects (Figure Sid).

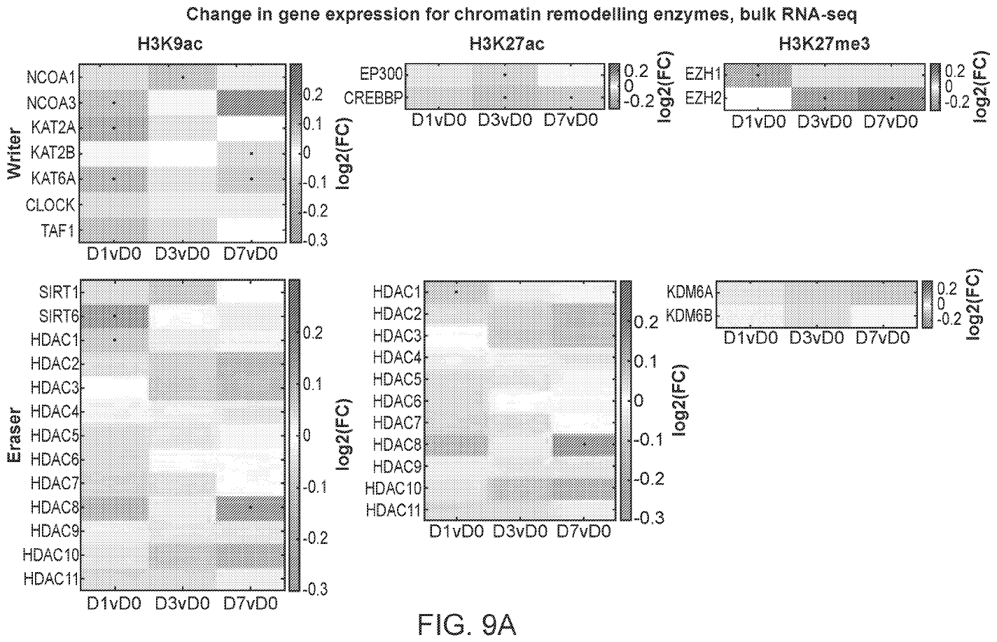

Blood transcriptomics data obtained from PBMCs at day 0 before and day 1, 3, 7 after vaccination from the same subjects (Hagan et al., 2019) revealed corresponding changes in the expression of histone modifying enzymes: histone acetylation writers, especially CREBBP/CBP (H3K27ac, H2BK5ac, H4K5ac,) and KAT6A (H3K9ac,) were significantly decreased after vaccination, while acetylation erasers (various HDACs) showed a trend towards increase (FIG. 9a). Histone acetylation is associated with active gene expression and especially H2BK5ac, H3K27ac, were shown to be highly predictive for global gene expression activity. Of note, the repressive histone mark, H3K27me3, which is also an antagonist of H3K27ac, was significantly increased in classical monocytes and myeloid dendritic cells (mDCs) (FIG. 1e) and the H3K27me3-writer, EZH2, showed increased expression in RNA-seq (FIG. 9a). Finally, PADI4 was previously shown to be an EpiTOF mark characteristic for myeloid cells and several reports show its involvement in monocyte and macrophage differentiation, activation and inflammation.

Next, we investigated the epigenomic reprogramming observed in myeloid cells at single-cell resolution (FIG. 1g). By performing sub-clustering and UMAP-based dimensionality reduction analysis of mDCs and classical monocytes using the H3K27ac, H2BK5ac, H4K5ac, H3K9ac, and PADI4 marks, we constructed the single-cell histone modification landscape. Importantly, in both cell types, single cells segregated according to vaccination time point with cells at day 0 and 1 clustering together on one side of the 2d space, and cells at day 30 occupying the opposite side (FIG. 1g). Cells at day 7 assumed an intermediate position and were spread between day 0 and day 30 clusters. In line with our previous findings, single cells at day 30 mostly had low levels of H3K27ac while cells at day 0 had high levels. Interesting, and undetected by the bulk kinetics analysis (FIG. 1f), cells at day 180 did not return to the baseline position occupied by day 0 cells but assumed an intermediate state with not fully restored H3K27ac levels (FIG. 1g). Together, these results point towards a concerted, potentially repressive, reprogramming of the myeloid epigenomic landscape that is driven by changes at the single-cell level.

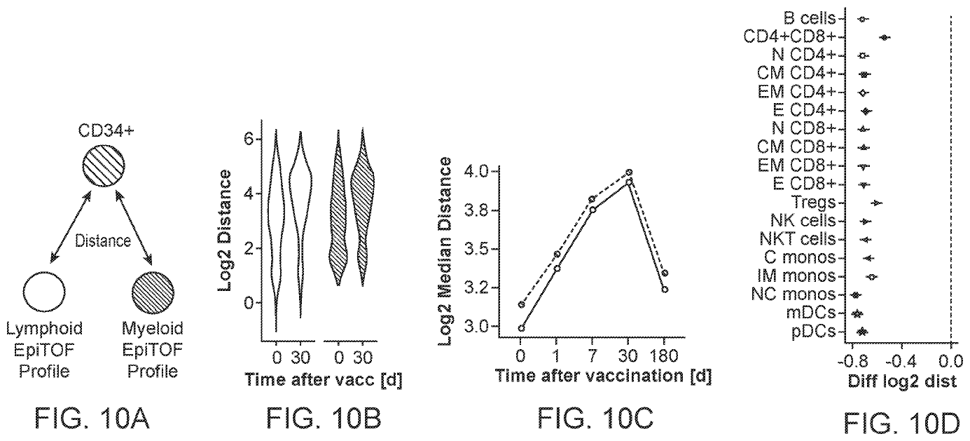

These observations raise the question of how persistent epigenetic changes that persist for up to 6 months, can be maintained in monocytes and mDCs are known to have a relatively short turnover. Recent studies indicate that such persistent changes in circulating myeloid cells is associated with persistent epigenetic changes in the hematopoietic stem and progenitor cell compartment in the bone marrow. To determine if this was also evident in the current study, we calculated the epigenomic distance to a consensus profile of differentiated lymphoid or myeloid cells (FIG. 10a). Interestingly, we detected multiple populations of CD34⁺ cells based on their epigenomic distances with minor populations showing relatively small distances to differentiated immune cells, possibly resembling pre-committed clones (FIG. 10b). After vaccination, the overall distance of CD34⁺ cells to either lymphoid or myeloid cells increased, and the fraction of potentially pre-committed progenitors were greatly reduced (FIG. 10b-d) indicating a potential shift of the stem cell pool towards an immature phenotype after vaccination. At day 180, the distances returned to their pre-vaccination state.

Together, our results demonstrate that vaccination with TIV leads to global epigenomic reprogramming of various immune cell subsets. In particular classical monocytes and dendritic cells are characterized by a concerted reduction in multiple histone acetylation marks and PADI4, that persists for up to 180 days, potentially indicating a repressed epigenomic state in these cells.

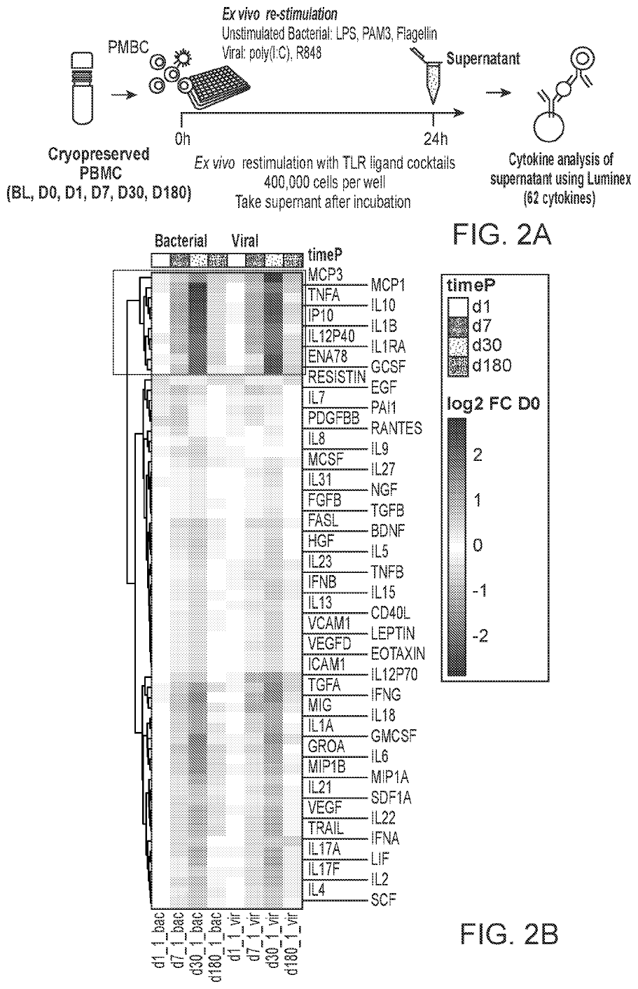

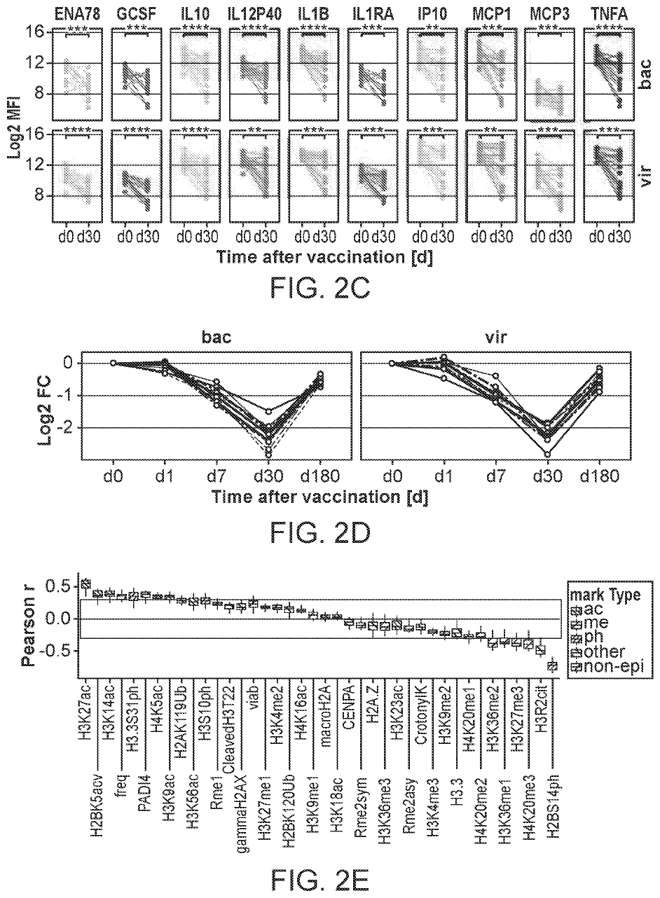

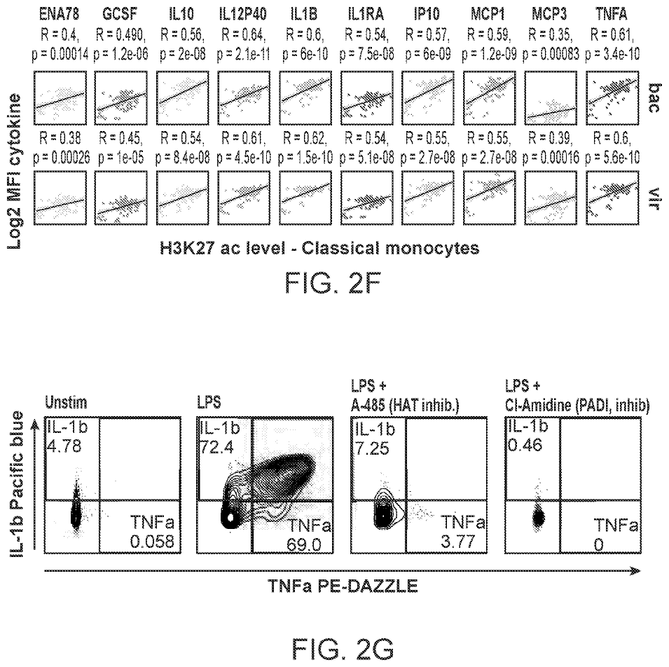

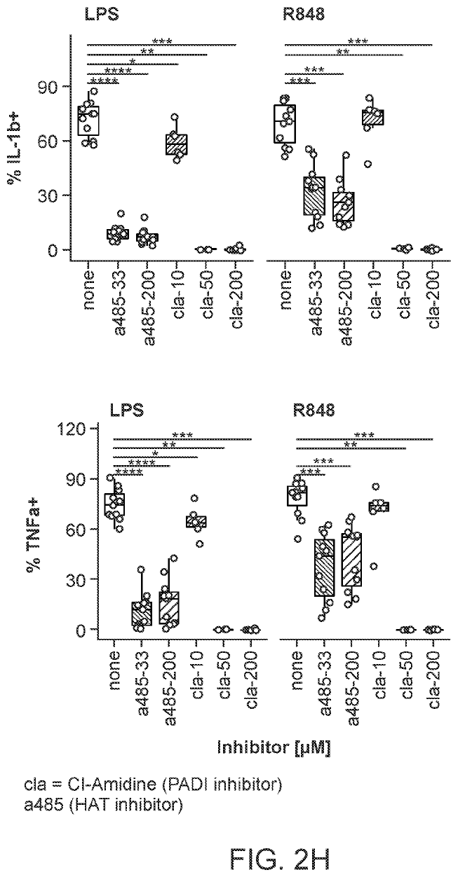

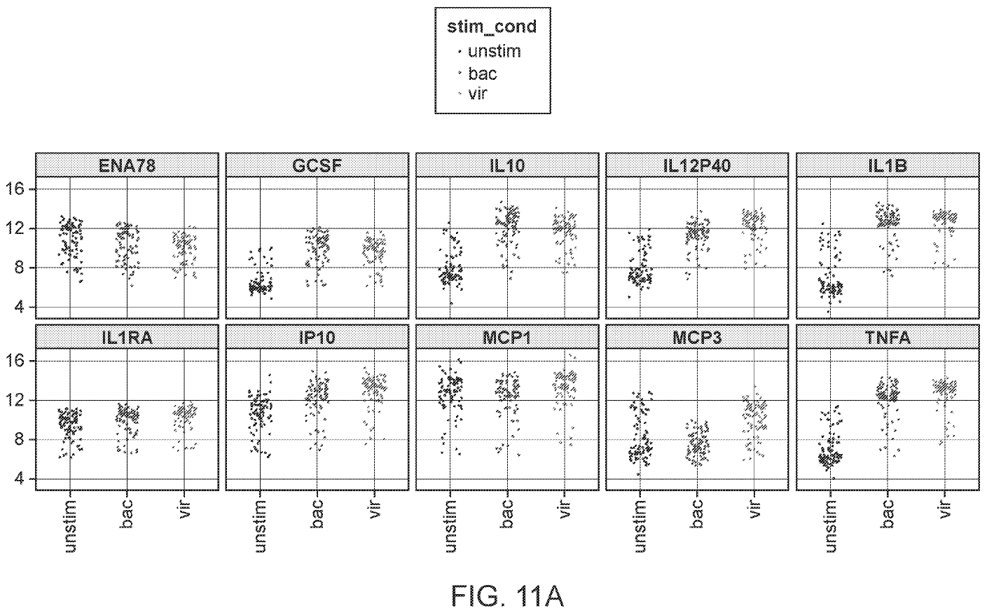

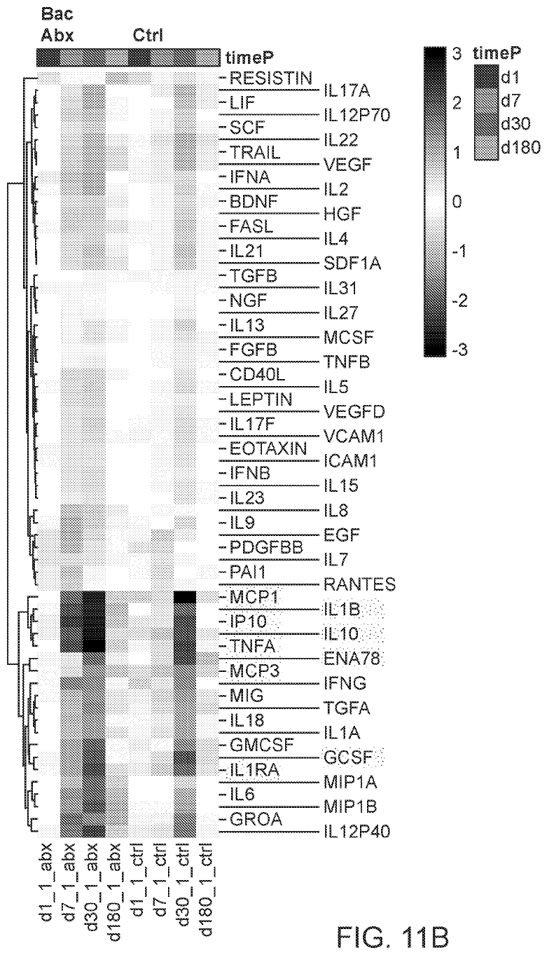

TIV induces persistent functional changes in innate immune cells. Given that both histone acetylation and PADI4 activity are associated with gene expression and monocyte function, we wondered whether the observed reduction in these marks at day 30 after TIV had any impact on myeloid cell function. To answer this question, we stimulated PBMCs from vaccinated individuals prior to vaccination, or at various time points after vaccination with cocktails of synthetic TLR ligands mimicking bacterial (LPS, Flagellin, Pam-3-Cys) or viral (pl:C, R848) pathogen-associated molecular pattern (FIG. 2a). After 24 h of stimulation, we measured the concentration of 62 secreted cytokines in culture supernatants using Luminex. To determine whether PBMCs from time points after vaccination showed any alterations in cytokine production, we calculated the relative change in cytokine concentration compared to DO (FIG. 2b). Indeed, using hierarchical clustering, we identified a subset of cytokines that displayed a significant reduction at day 30 after vaccination (FIG. 2b red box, c). These cytokines include TNF-α, IL-1b, IL-1 RA, IL-12, and IL-10, the monocytic chemokines MCP1, MCPS, ENA78 (CXCL5), and IP-10 (CXCL10), as well as the monocyte growth factor GCSF. Similar to the epigenomic changes, cytokine levels begin to fall around day 1 to 7 after vaccination, reaching a nadir at day 30, and almost returning to baseline levels at day 180 (FIG. 2d). All of these cytokines were strongly induced by both TLR cocktails (FIG. 11a) and a reduction relative to DO was observed in both antibiotics and control subjects (FIG. 11b).

Next, we investigated whether there is a direct relationship between global histone modification levels and TLR-induced cytokine production. We used pairwise correlation analysis to correlate the cytokine concentrations in a sample with the EpiTOF histone modification levels in classical monocytes as well was with monocyte frequency in the PBMC sample and cell viability (FIG. 2e). Strikingly, the histone acetylation marks previously identified in FIG. 1c, especially H3K27ac and PADI4, showed strong positive correlation with cytokine production (FIG. 2e, f). In contrast, H2BS14ph and several repressive methylation marks, including H3K27me3 and H4K2Ome3, were negatively correlated with cytokine production (FIG. 2e).

Next, we determined whether there is a causal relationship between the observed reduction in H3K27ac histone acetylation and PADI4 levels, and the diminished cytokine production. We conducted an ex-vivo stimulation experiment using specific inhibitors for the histone acetyl transferases CBP/p300 (A-485, inhibits acetylation at H3K27, H2BK5, and H4K5, and PADI4 (CI-Amidine) followed by stimulation with synthetic TLR ligands. We also used trichostatin A (TSA, enhances histone acetylation via inhibition of HDACs, as a positive control for detecting histone acetylation. Using flow cytometry, we assessed expression of H3K27ac and the intracellular accumulation of IL-1β and TNFα. As expected, treatment with the histone acetyl transferase inhibitor A-485 leads to a concentration-dependent decrease in global histone H3K27ac levels in classical monocytes while treatment with the HDAC inhibitor TSA leads to a concentration-dependent increase. Furthermore, treatment with the PADI4 inhibitor CI-Amidine lead to similar reductions in H3K27ac in line with tight correlation of PADI4 and H3K27ac levels in EpiTOF and the previously observed ability of PADI4 to regulate CBP/P300 (Lee et al., 2005). Notably, none of these inhibitors had an effect on cell viability (Data not shown). Next, we asked whether inhibition of CBP/P300 and PADI4 has an impact on cytokine production. Indeed, treatment with A-485 led to a major diminution in the frequency of IL-1b and TNFα positive monocytes after stimulation with LPS or R848 (FIG. 2g, h). CI-Amidine treatment, strikingly, led to a complete abrogation of cytokine production in these cells (FIG. 2h).

Together, these results demonstrate that TIV vaccination induces reduced innate immune cells functionality and this reduction was correlated and causally linked with changes in the epigenomic landscape of classical monocyte.

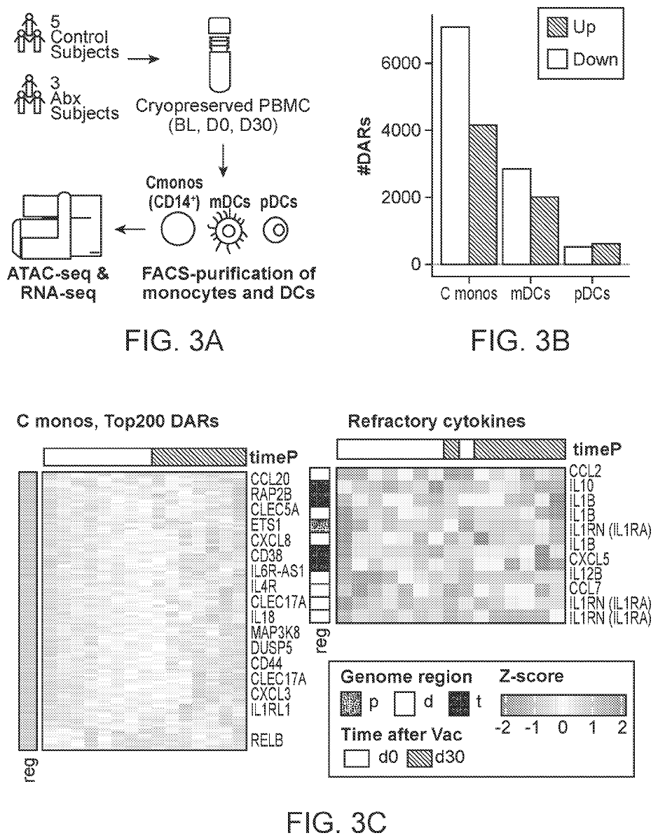

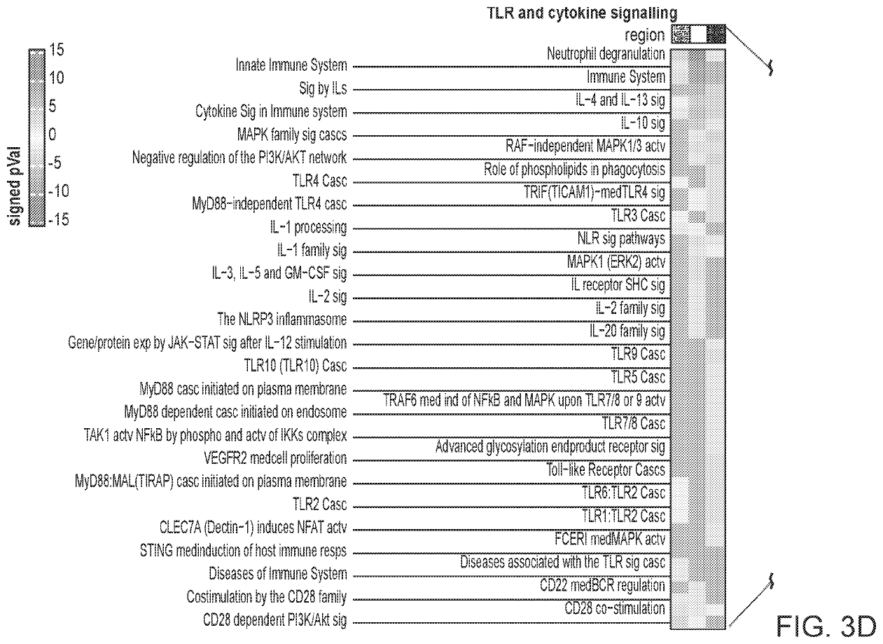

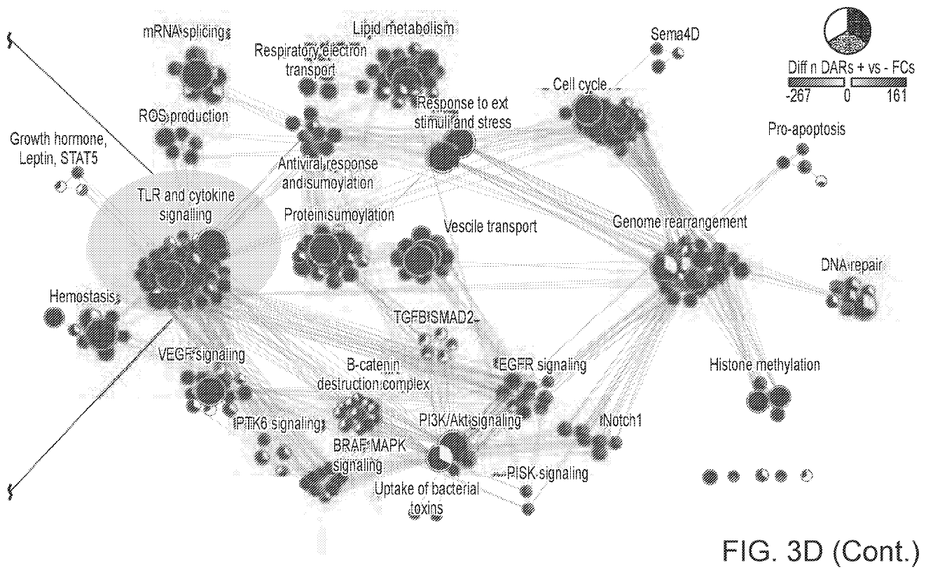

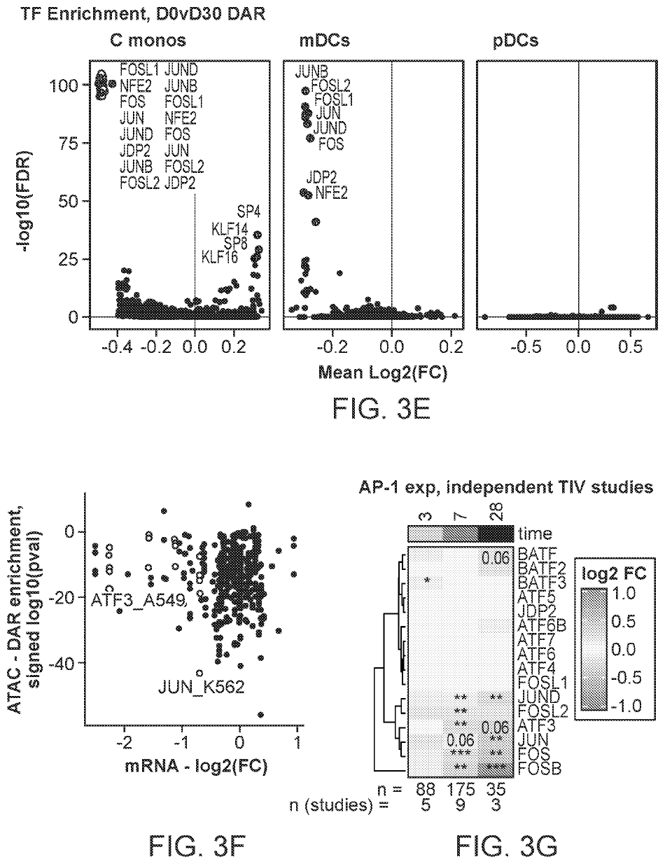

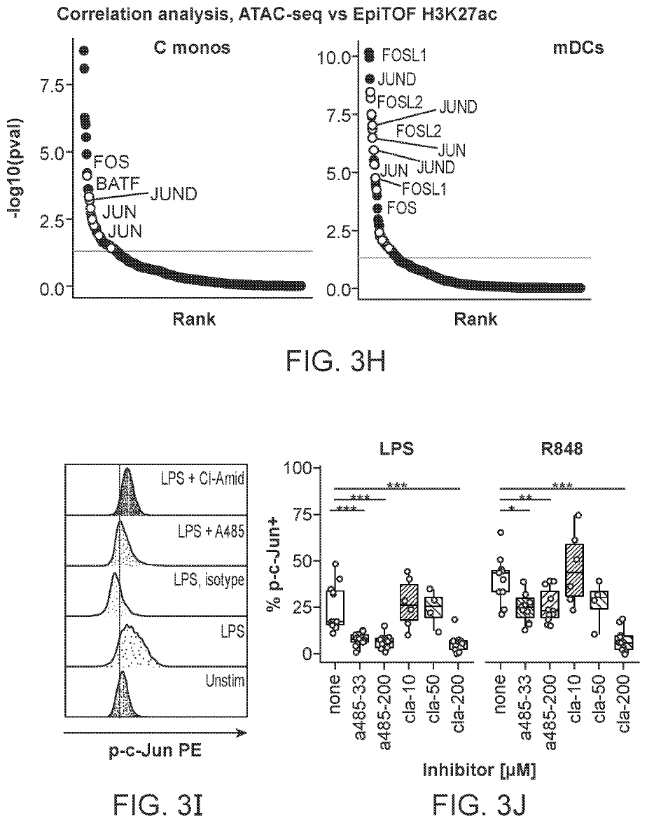

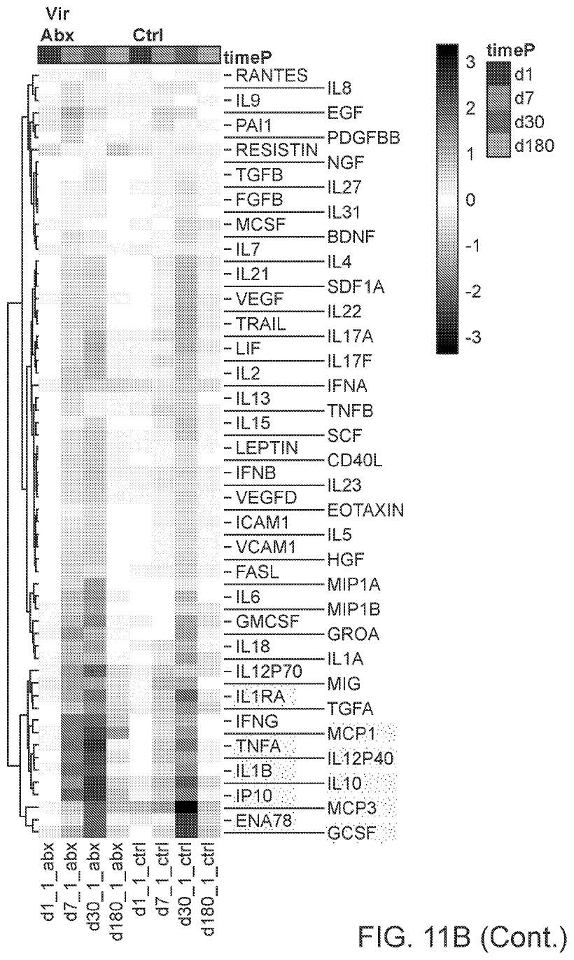

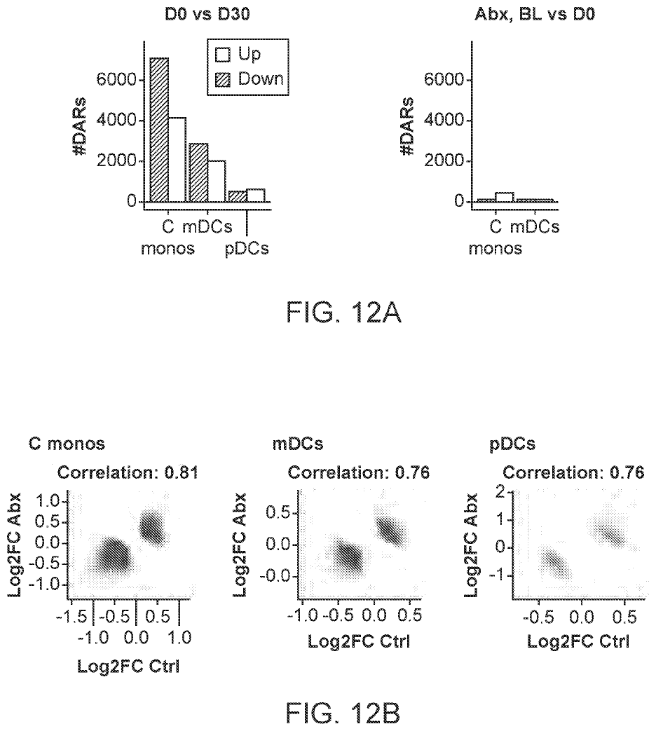

Vaccination against seasonal influenza induces reduced chromatin accessibility of AP-1 targeted loci in myeloid cells. To gain greater insight into the epigenomic changes induced by vaccination, we conducted ATAC-seq analysis of FACS purified innate immune cell subsets before and after vaccination (FIG. 3a). After preprocessing, we retained a high-quality dataset of 57 unique samples. To identify the molecular targets of the TIV-induced epigenomic changes, we determined genomic regions with significantly changed chromatin accessibility at day 30 after vaccination compared to day 0 before. Overall, we detected more than 10,000 differentially accessible regions (DARs) in CD14⁺ monocytes and about 4,500 DARs in mDCs while pDCs showed only minor changes (FIG. 3b). In line with reduced histone acetylation levels detected by EpiTOF, the majority of DARs in monocytes and mDCs showed a reduction in chromatin accessibility indicating reduced gene activity (FIG. 3b). In contrast, comparing samples from day −21 before antibiotics treatment and day 0 during antibiotics treatment showed no profound change in chromatin accessibility (FIG. 12a) and D0vD30 DARs correlated well between antibiotics and control subjects (FIG. 12b). Among the top 200 DARs in CD14⁺ monocytes, we identified many immune-related genes with reduced accessibility, including several cytokines and chemokines and their associated receptors (IL18, CCL20, CXCL8, CXCL3, IL4R, IL6R-AS1), pathogen recognition receptors (CLEC5A, CLEC17A), and adhesion molecules (CD44, CD38) (FIG. 3c). We also observed reduced accessibility in regions coding for molecules associated with Ras-MAPK-AP-1 signaling (RAP2B, ETS1, MAP3K8, DUSP5) (FIG. 3c). Importantly, genomic loci associated with seven of the ten cytokines with reduced concentrations during ex-vivo restimulation showed reduced accessibility (FIG. 2, FIG. 3c, right panel). Interestingly, these reduced DARs were predominantly located in non-promoter regions (FIG. 3c) suggesting the involvement of distal regulatory elements such as enhancers. Pathway analysis followed by network analysis of DARs in CD14⁺ monocytes determined two dominant biological themes: TLR and cytokine signaling, and genome rearrangement (FIG. 3d). The TLR and cytokine cluster was dominated by pathways with mostly reduced chromatin accessibility while terms in the genome rearrangement cluster were mixed. Notably, DARs associated with signaling pathways around Ras and MAPK signaling were enriched as well.

Next, to identify regulatory patterns, we determined whether the identified DARs in each cell type were enriched for transcription factor (TFs) binding motifs. Indeed, we observed an enrichment for bZIP TFs of the AP-1 family including c-Jun and c-Fos in DARs of monocytes and mDCs, and DARs carrying such a motif showed on average a reduction in chromatin accessibility after vaccination, especially in non-promoter regions (FIG. 3e). Gene set analysis of the DARs in classical monocytes further confirmed this finding and showed strong enrichment for target genes of c-Jun in DARs with reduced accessibility at day 30 (FIG. 3f). In addition, we observed reduced expression of several AP-1 family members, including c-Jun, at day 30 after vaccination (FIG. 3f). Using bulk transcriptomics from previous systems vaccinology studies, we confirmed the reduced c-Jun expression in up to 9 independent flu vaccine studies and additionally identified a reduction in expression of the AP-1 members JUND, ATF3, FOS, FOSL2, and FOSB (FIG. 3g). Similar to the histone acetylation changes, the reduction in AP-1 TF expression was first detected at day 7 after vaccination and was most pronounced at day 28 (FIG. 3g).

Based on this observation, we asked whether the observed reduction in AP-1 accessibility is related to the reduced levels of histone acetylation. To test this, we correlated the normalized accessibility levels of all genomic regions in every sample to histone mark levels in classical monocytes or mDCs of the same sample. Using enrichment analysis on the highly correlated peaks (cor coef>0.5), we identified a significant enrichment of target genes for multiple AP-1 family members, including c-Fos and c-Jun (FIG. 3h).

Together, these findings suggest the possibility of a causal link between reduced histone acetylation/PAD14 and reduced AP-1 accessibility. Indeed, previous studies described a direct physical interaction and functional co-dependence between AP-1 and the histone acetyl transferases CBP/P300. To investigate whether AP-1 activity and histone acetylation are also functionally linked in classical monocytes, we conducted an ex-vivo stimulation experiment using the same specific inhibitors of histone acetylation and PADI activity as in FIG. 2. To gauge AP-1 activity, we used an intracellular antibody staining able to detect the activated, phosphorylated form of c-Jun. While treatment with LPS or R848 alone induced a robust upregulation of p-c-Jun that can be readily detected by flow cytometry (FIG. 3i, j), pre-treatment with A-485 or CI-Amidine, which lead to reduced histone acetylation, abolished c-Jun activation completely (FIG. 3i, j).

Together, these results demonstrate that TIV also leads to reduced chromatin accessibility in classical monocytes and mDCs. Reduced accessibility is primarily found in regions that are associated with TLR- and cytokine-related genes and regions that carry the AP-1 TF binding motif. Furthermore, HAT/PADI activity is causally linked to AP-1 activation.

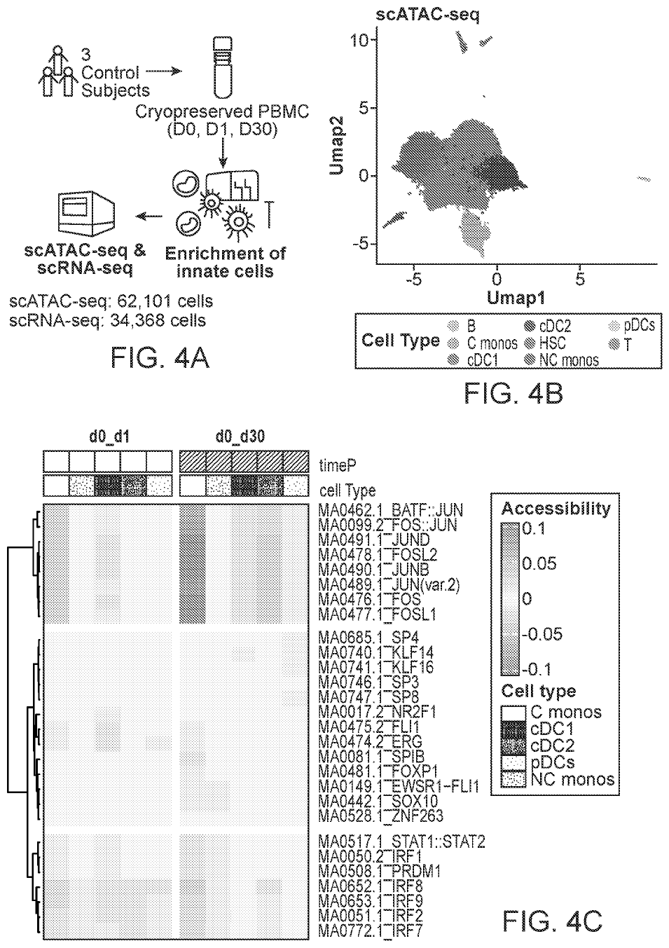

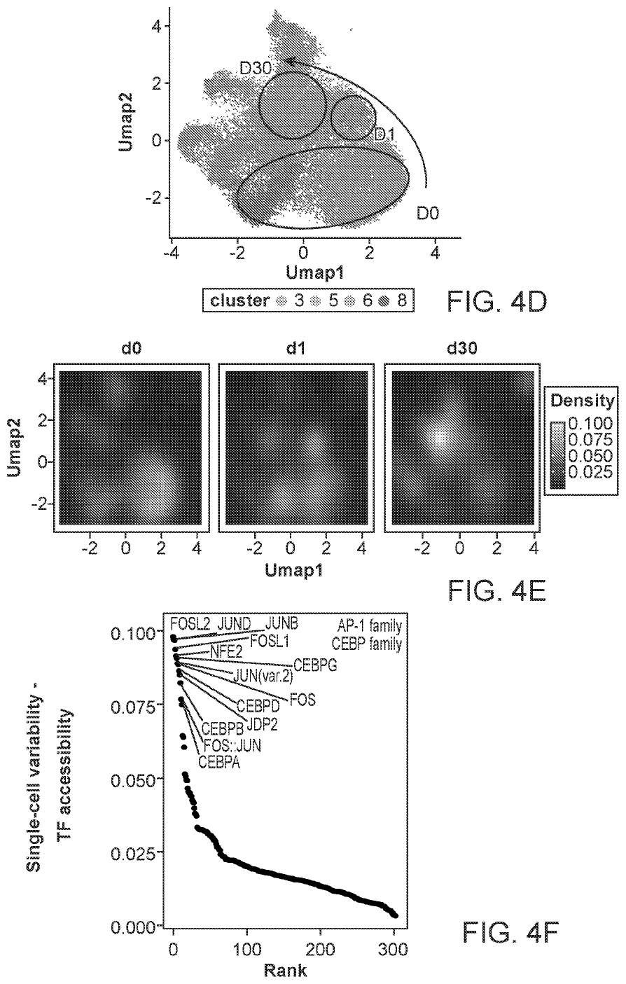

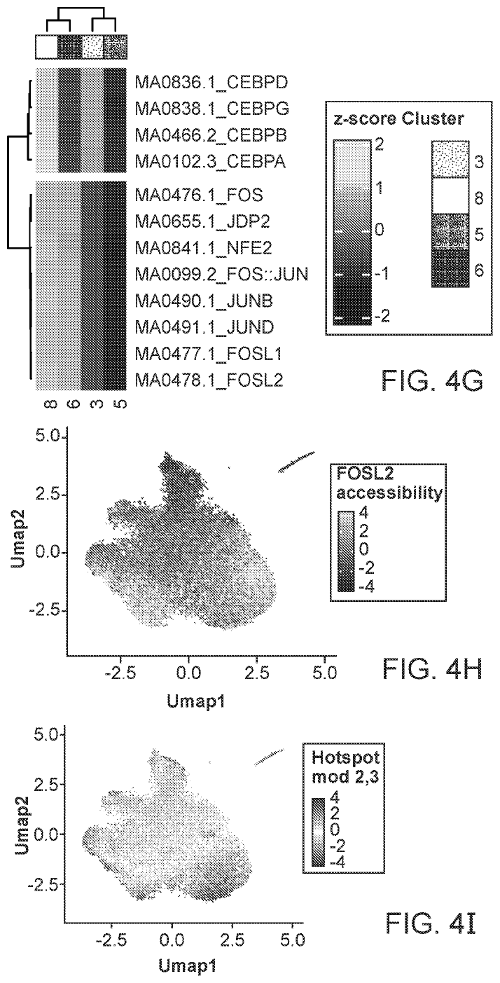

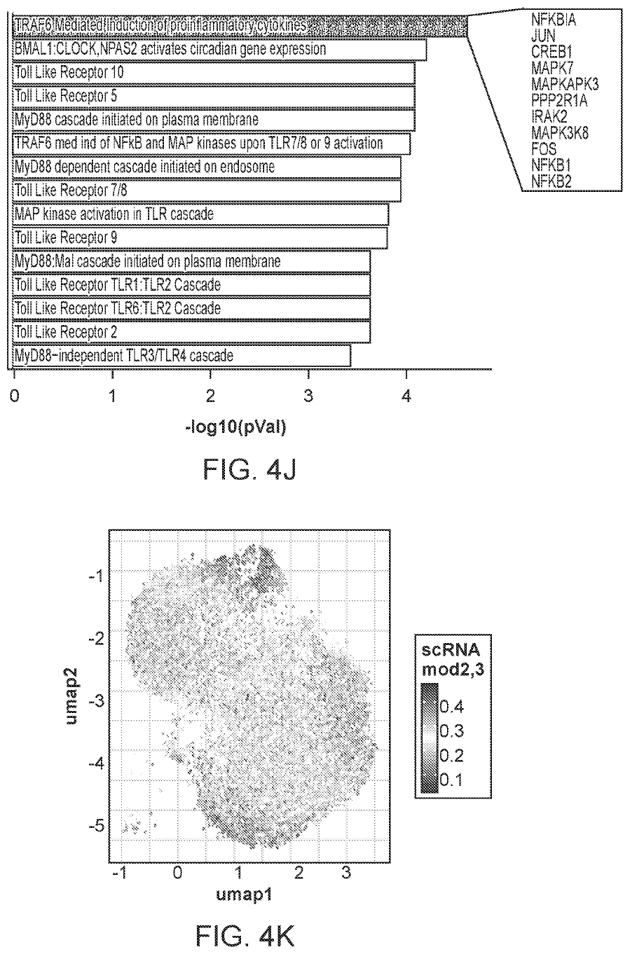

Single-cell epigenomic and transcriptional landscape of the innate response to seasonal influenza vaccination. Previous studies, using transcriptomics and proteomics approaches, detected ample heterogeneity within monocyte and dendritic cell populations at steady state. However, it is unclear how this heterogeneity affects the epigenomic landscape in these cells and their response to vaccination. To answer this question, we used scATAC-seq and scRNA-seq and constructed the single-cell landscape of the innate immune response to TIV at the epigenomic and transcriptional level. PBMCs from vaccinated individuals were isolated at day 0, 1, and 30 and enriched for DC subsets using flow cytometry and analyzed using droplet-based single-cell gene expression and chromatin accessibility profiling (FIG. 4a). After initial pre-processing, we obtained chromatin accessibility data from 62,101 cells with an average of 4,126 uniquely accessible fragments. These cells displayed the canonical fragment size distribution and showed high signal-to-noise ratio at transcription start sites. Using UMAP representation and chromVAR TF deviation patterns, we generated an epigenomic map of the innate immune system and identified clusters for all major innate immune cell subsets, including classical and non-classical monocytes, mDCs, and pDCs (FIG. 4b). In parallel, we used the scRNA-seq data to construct a gene expression map. After pre-processing, we retained 34,368 high-quality transcriptomes with an average of 2,477 genes and 8,951 unique transcripts detected per cell. UMAP representation in combination with clustering allowed us to identify all major innate immune cell subsets. All immune cell subsets were detected in all vaccine conditions and subjects.

First, we used chromVAR to determine the TIV-induced changes in TF chromatin accessibility. AP-1 accessibility was strongly reduced at day 30 after vaccination in classical monocytes and mDCs (both cDC1 and cDC2) (FIG. 4c) confirming our findings with bulk ATAC-seq (FIG. 3). In addition, using the single-cell dataset, we observed that the reduction in AP-1 accessibility starts early, at day 1 after vaccination (FIG. 4c) suggesting that the TIV-induced epigenomic reprogramming is imprinted during the acute phase of the vaccine response. On the gene level, we observed a reduction in the expression of multiple AP-1 members including ATF3, JUND, JUNB, FOS, and FOSL2.

Next, we determined the impact of cellular heterogeneity on TIV-induced epigenomic changes. Subclustering analysis of classical monocytes revealed the presence of four distinct populations based on chromatin accessibility (FIG. 4d) with different temporal patterns (FIG. 4e): while clusters 6 and 8 dominated the classical monocyte pool at day 0, most cells at day 30 belonged to cluster 5 (FIG. 4d, e). Notably, the observed heterogeneity between the classical monocyte populations was driven by differences in AP-1 and, to a lower extend, CEBP accessibility (FIG. 4f). While the dominating clusters at day 0 (cluster 6 and 8) were high in AP-1 accessibility, cells in cluster 5, which was predominantly found at day 30, were low in AP-1 (FIG. 4g, h). Cells in cluster 3 exhibited intermediate AP-1 and CEBP accessibility (FIG. 4g) and their relative abundance was stable throughout vaccination (FIG. 4e). Using, the Hotspot algorithm (DeTomaso and Yosef, 2020), we determined a set of genomic regions underlying the observed heterogeneity (FIG. 4i). This set was enriched for regions associated with the production of proinflammatory cytokines and TLR signaling (FIG. 4j), and included regions associated with the AP-1 members FOS and JUN, multiple MAP kinases, and NFKB. Importantly, cells with high AP-1 accessibility using the motif-based chromVAR analysis, also displayed high accessibility at these inflammatory genome regions (FIG. 4h, i). Finally, using the scRNA-seq dataset, we determined the cellular heterogeneity at the transcriptional level. Although genes in the Hotspot module varied in their expression between single classical monocytes, this heterogeneity was less distinct compared to the epigenomic landscape (FIG. 4k).

Together, these findings demonstrate that AP-1 accessibility drives epigenomic heterogeneity within the pool of classical monocytes and defines epigenomic subclusters. Importantly, changes in the relative abundance of these epigenomic subclusters underly the observed global reduction in AP-1 accessibility after vaccination and a population of AP-1 low, seemingly less inflammatory cells dominated the monocyte pool at day 30.

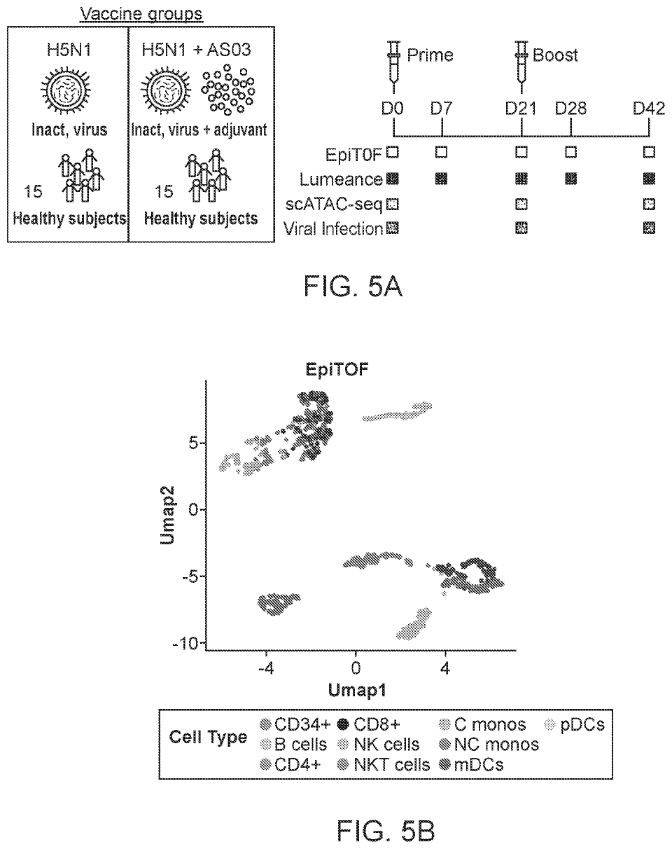

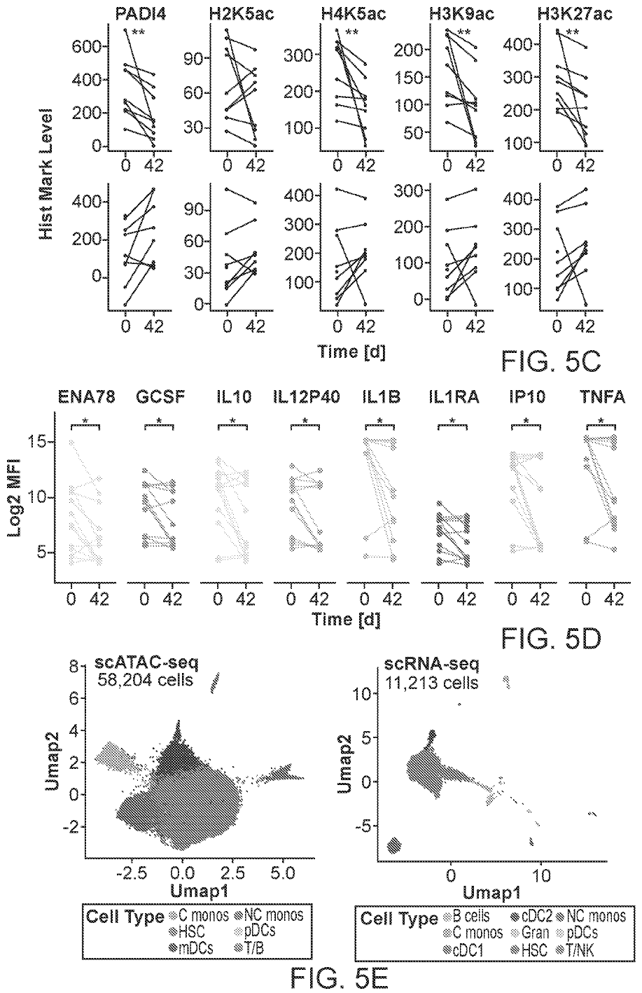

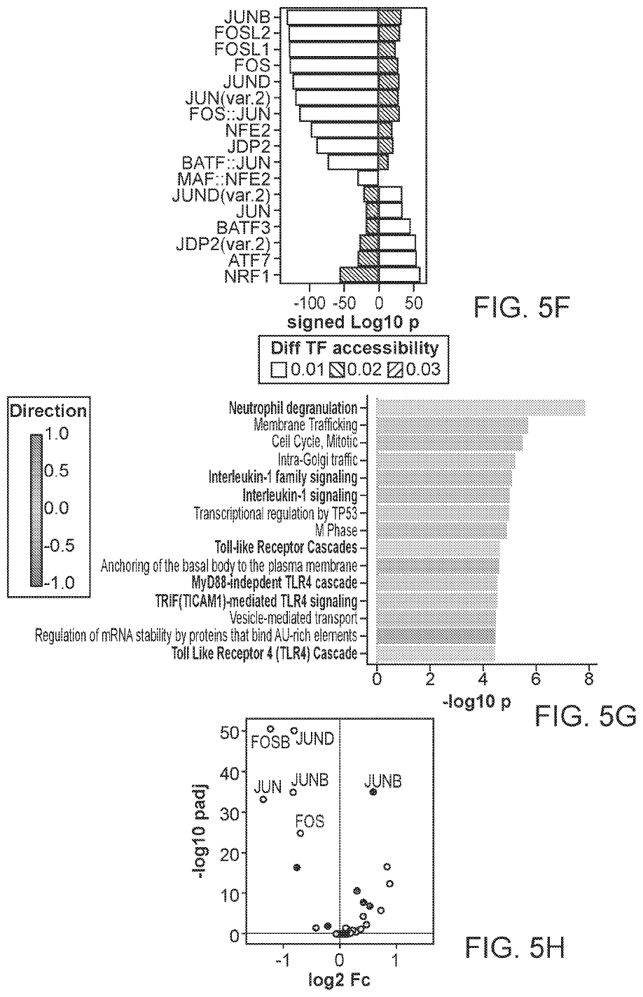

AS03 adjuvanted H5N1 influenza vaccine induces reduced chromatin accessibility of AP-1 loci in myeloid cells. The effects of the inactivated seasonal influenza vaccination in inducing reduced chromatic accessibility of AP-1 loci, and reduced H3K27ac and refractoriness to TLR stimulation by myeloid cells, was unexpected and seemingly at odds with a prior study with the live attenuated BCG vaccination that showed enhanced and persistent innate responses to vaccination, termed “trained immunity”. This raised the possibility that the live BCG vaccine delivered potent adjuvant signals that stimulated persistent epigenomic changes in myeloid cells, whereas the seasonal influenza vaccine used in the current study being an inactivated vaccine devoid of an adjuvant, was unable to stimulate trained immunity, but rather induced a form of trained tolerance. Therefore, we wondered how the addition of an adjuvant to an inactivated influenza vaccine would affect the epigenomic immune cell landscape. Adjuvants are stimulants designed to strongly activate the innate immune system during vaccination. AS03 is a squalene-based adjuvant and induces strong innate and adaptive immune responses and is included in the licensed H5N1 avian influenza vaccine. Here, we investigate the effect of AS03 on the epigenomic immune cell landscape in a cohort of healthy individuals that were vaccinated with an inactivated H5N1 influenza vaccine administered with or without AS03 (FIG. 5a). The vaccine was administered in a prime-boost regiment and individuals received injections at day 0 and day 21.

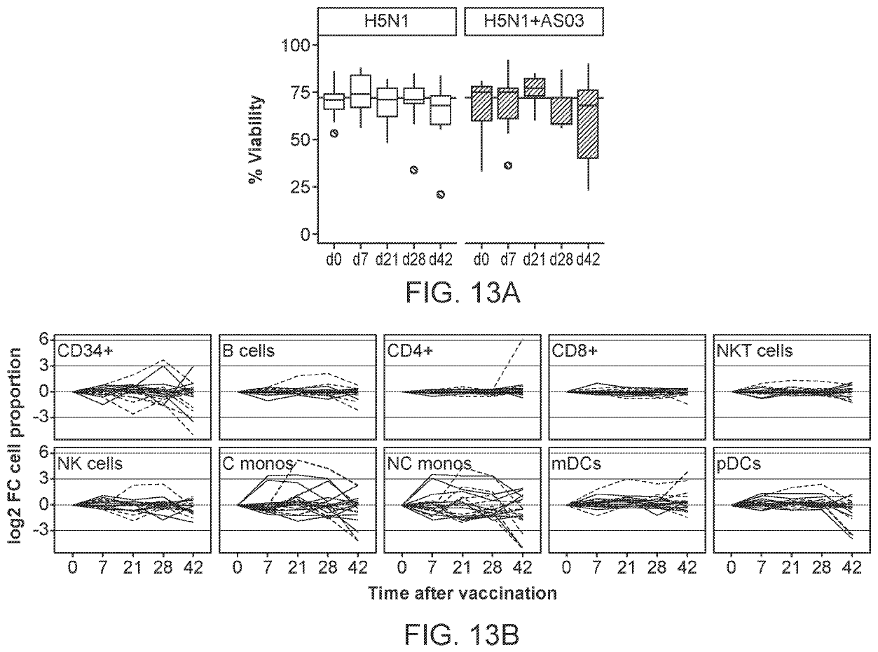

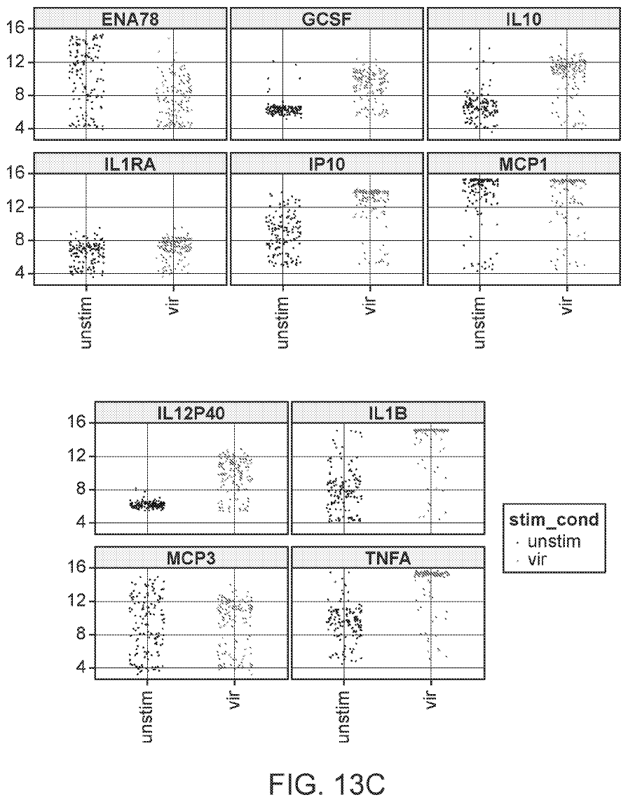

First, we asked how the presence of AS03 would affect the vaccine-induced chromatin mark changes observed after vaccination with TIV. Using EpiTOF, we analyzed PBMC samples from 18 vaccinated subjects (9 H5N1, 9 H5N1+AS03) at day 0, 7, 21, 28, and 42 and constructed the histone modification profile landscape (FIG. 5b, FIG. 13a). Unexpectedly, we observed that vaccination with H5N1+AS03 induced a significant reduction in H3K27ac, H4K5ac, H3K9ac, and PADI4 in classical monocytes, four of the five highly correlated marks associated with myeloid reprogramming after TIV (FIG. 5c). In contrast, vaccination with H5N1 alone did not induce significant changes in these chromatin marks. In line with these findings, in the H5N1+AS03 group but not the H5N1 group, we also observed significantly reduced production of most of the innate cytokines and chemokines that were diminished after vaccination with TIV (FIG. 5d). Notably, we did not detect a change in the frequency or viability of classical monocytes within the PBMC mixture and all of these cytokines were strongly induced by TLR stimulation (FIG. 13a, b).

Next, we analyzed subjects from this cohort using scATAC-seq and scRNA-seq. PBMC samples from 4 vaccinated individuals (2 H5N1, 2 H5N1+AS03) at day 0, 21, and 42 were enriched for DC subsets using flow cytometry and analyzed using droplet-based single-cell gene expression and chromatin accessibility profiling (FIG. 5a). After initial pre-processing, we obtained high quality chromatin accessibility data from 58,204 cells with an average of 2,745 uniquely accessible fragments which we used to generate an epigenomic map of the single immune cell landscape during H5N1 vaccination (FIG. 5e). In parallel, we used the scRNA-seq data to construct a gene expression map of the single immune cell landscape. We retained 11,213 high-quality transcriptomes with an average of 2,462 genes and 9,569 unique transcripts detected per cell and identified all major innate immune cell subsets (FIG. 5c). The different immune cell subsets were evenly distributed over all vaccine conditions and time points.

Notably, using the scATAC-seq data, we observed a significant reduction in AP-1 accessibility in H5N1+AS03 but not H5N1 alone (FIG. 5f). To further investigate the nature of the epigenomic changes after H5N1+AS03, we determined the differentially accessible regions at day 42 after vaccination compared to day 0 using a logistic regression model that corrects for library-size differences. Using overrepresentation analysis, we found that, similar to TIV, the predominantly negative DARs were enriched for TLR-, and cytokine-signaling pathways as well as innate immune activity (FIG. 5g). Additionally, using the scRNA-seq data, we observed a reduction in the expression of multiple AP-1 family members, including c-Fos and c-Jun as observed after vaccination with TIV (FIG. 5h). Together, these findings suggest that vaccination with H5N1+AS03 induces epigenomic changes very similar to those observed after vaccination with TIV while vaccination with H5N1 alone only causes minor alterations to the epigenomic landscape.

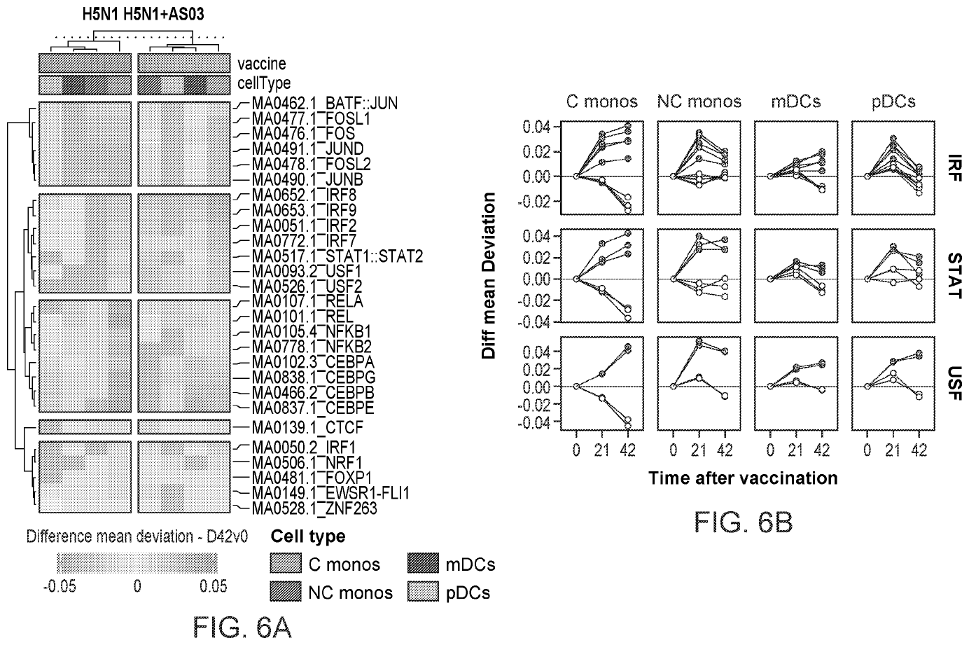

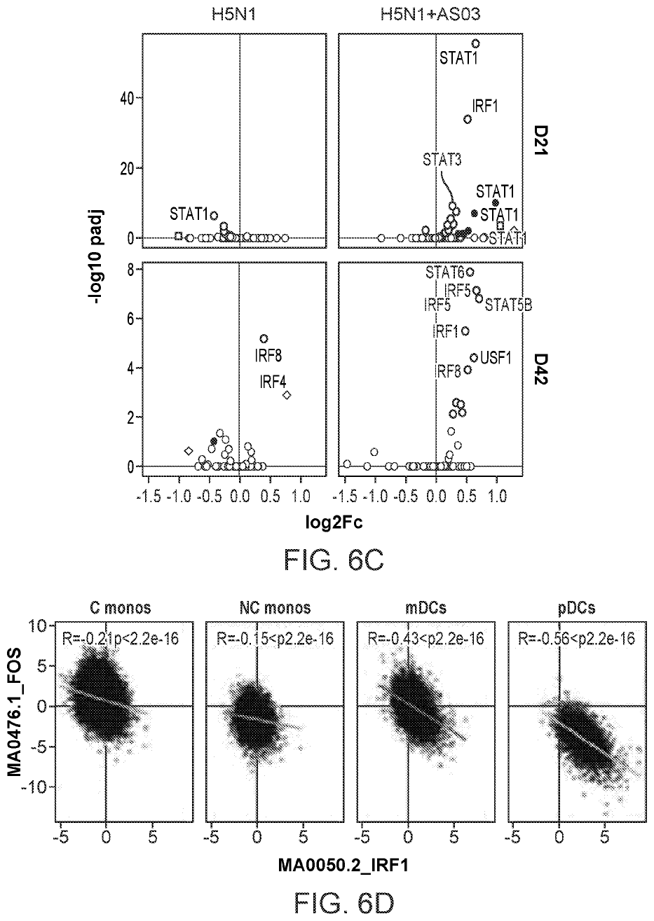

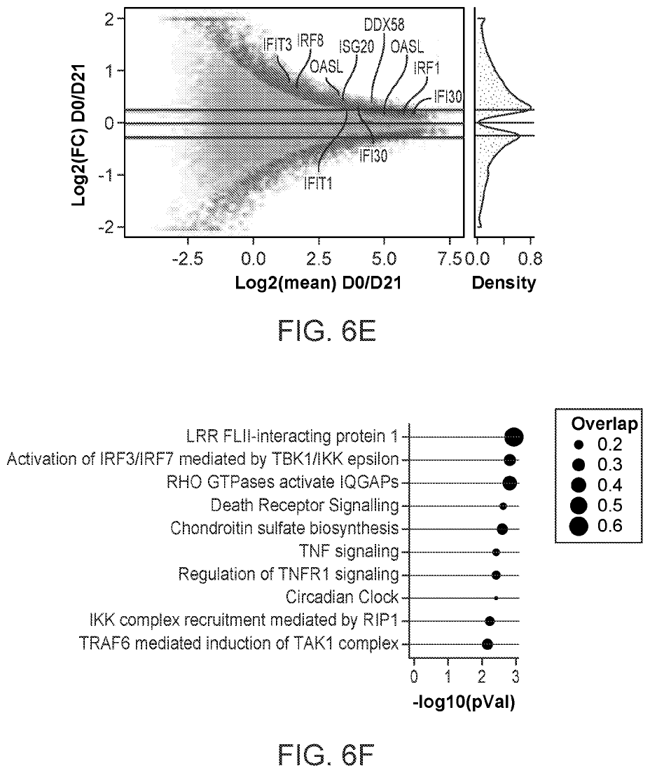

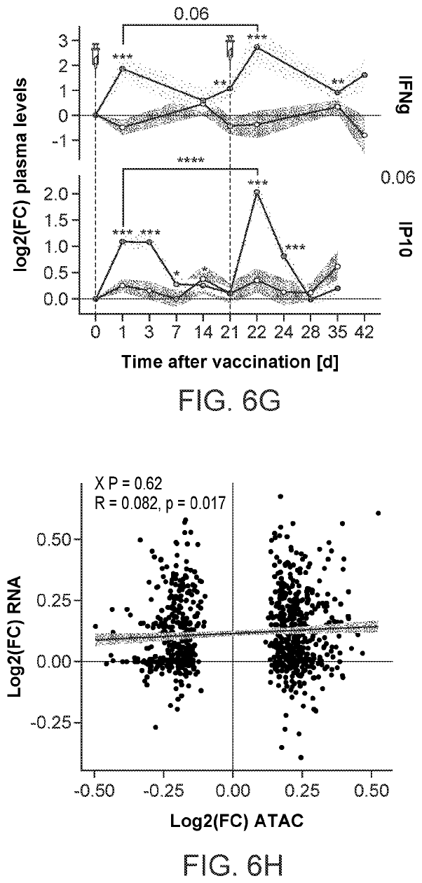

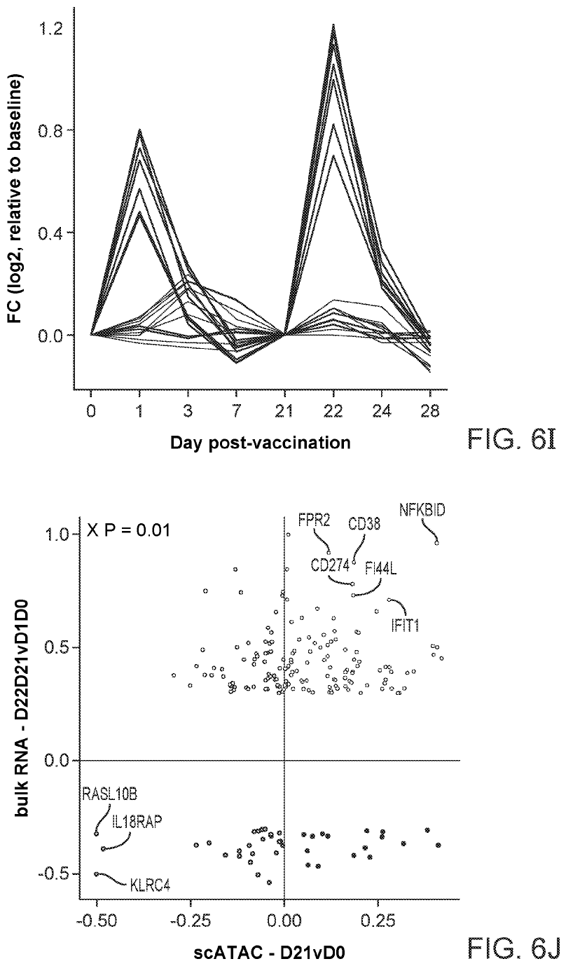

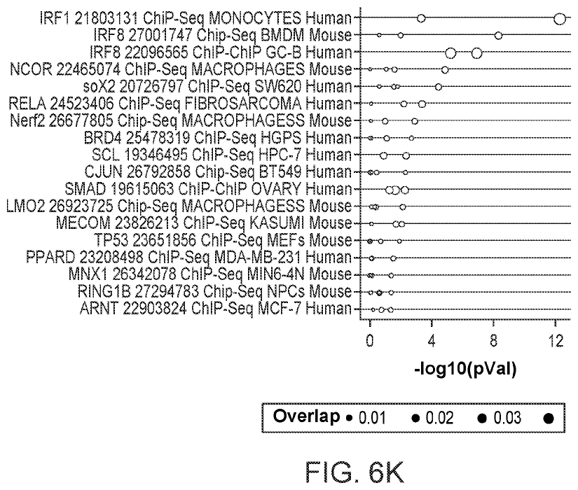

AS03 adjuvanted H5N1 influenza vaccine induces enhanced chromatin accessibility of the antiviral response loci. Despite the reduction in AP-1 accessibility, we observed an increase in chromatin accessibility at day 42 compared to day 0 for several TFs of the interferon-response factor (IRF) and STAT families (FIG. 6a). These changes were observed in innate cell populations of subjects vaccinated with H5N1+AS03, but not with H5N1 alone. Further analysis of the kinetics revealed that these IRF- and STAT-related changes are already present after administration of the first vaccine shot at day 21 (FIG. 6b). Using the scRNA-seq dataset, we compared the expression or IRF and STAT family TFs before and after prime (day 21) or boost (day 42) vaccination. We observed significant increases in the expression of IRF1, and STAT1 in multiple innate immune cell subsets after vaccination with H5N1+AS03, but not with H5N1 alone (FIG. 6c). Notably, at a single-cell level, IRF accessibility was generally negatively correlated with AP-1 accessibility (FIG. 6d), especially in dendritic cells. Next, we determined the log fold change in chromatin accessibility for peaks containing the IRF1 binding motif (FIG. 6e). Indeed, we observed a significant change in accessibility in many peaks, most of which showed increased accessibility (FIG. 6e). Importantly, amongst the genes with increased accessibility, we identified many interferon- and antiviral-related genes, including DDX58 (encoding the viral detector RIG-I), several interferon response genes (IFIT1, IFIT3, IFI30, ISG20, OASL), as well as the transcription factors IRF1 and IRF8. Enrichment analysis further demonstrated an enrichment of genes related to antiviral immunity (FIG. 6d). IRF1, together with STAT1 and IRF8, orchestrates monocyte polarization in response to interferon gamma exposure (Langlais et al., 2016) and IFN signaling, via JAK/TYK, leads to phosphorylation of IRF and STAT TFs (Tamura et al., 2008). Indeed, we observed an increase in IFN gamma levels in plasma of vaccinated subjects immediately after prime and boost vaccination with H5N1+AS03, but not with H5N1 alone (FIG. 6g). The levels of IP10, a cytokine that is produced by monocytes in response to IFN signaling, were elevated, too (FIG. 6g). This might suggest that IFN signaling could have induced the increased IRF accessibility.

Next, we determined whether the observed epigenomic changes were translated to changes in gene expression. We correlated the change in accessibility for significantly changed peaks carrying the IRF1 motifs with the change in gene expression for the same gene (FIG. 6h). Notably, we detected a significant but weak positive association between changes in accessibility and gene expression (R=0.082, p=0.017) indicating that the increased accessibility has limited impact on homeostatic gene expression in these cells. Instead, it might be that the changes in chromatin accessibility enhance the induced response to viral stimuli. To test this hypothesis, we analyzed bulk RNA-seq data from 50 (16 H5N1, 34 H5N1+AS03) vaccinated subjects at time points before and after the prime (days 0, 1, 3, 7) and booster (days 21, 22, 24, 28) vaccination. As expected, antiviral- and interferon-related genes were upregulated at day one after each vaccination, especially in the group that received H5N1+AS03 (FIG. 6i). Importantly, subjects receiving a H5N1+AS03 booster vaccination (day 22 vs day 21) displayed even higher levels of antiviral gene expression compared with the response to the prime vaccine (day 1 vs day 0) (FIG. 6i). The booster vaccine was given at a time when the chromatin accessibility landscape of the innate immune system was altered suggesting that the increased accessibility in IRF loci might enable the enhanced response to the booster vaccine. To further test this hypothesis, we compared the increase in gene expression of antiviral- and interferon-related genes during booster compared to prime with the change in chromatin accessibility at day 21 compared to day 0 (FIG. 6j). Indeed, we observed a highly significant association between both variables (Chi-square p-value=0.01) and most genes with increased expression after booster vaccination also showed increased chromatin accessibility at the time the booster vaccine was administered. Genes with increased accessibility and enhanced expression were enriched for IRF1 transcription factor target genes (FIG. 6k). In line with these observations, we also observed elevated levels of IP-10 and IFN gamma in plasma of individuals after the booster compared to prime vaccination (FIG. 6g).

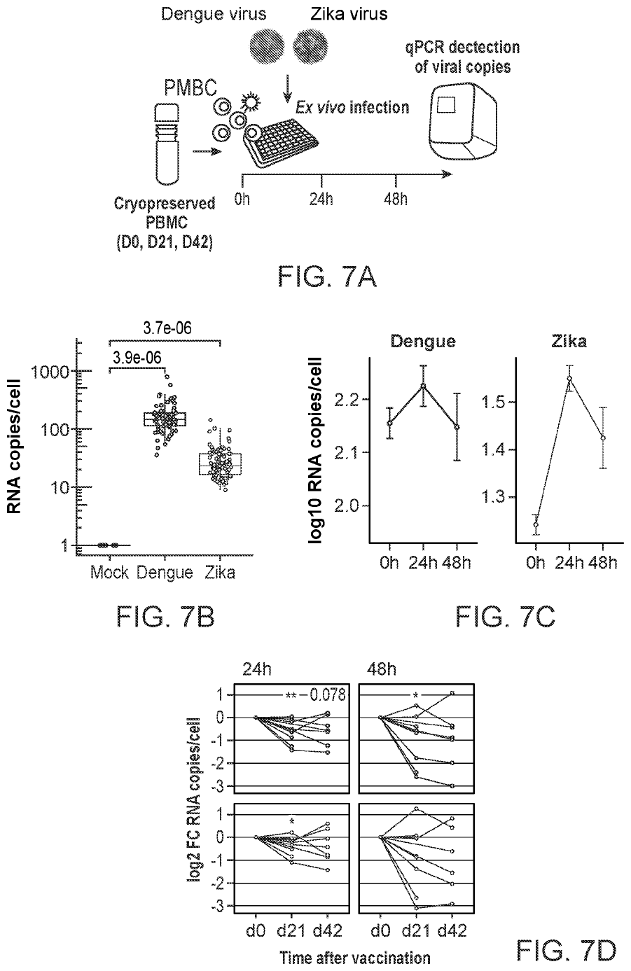

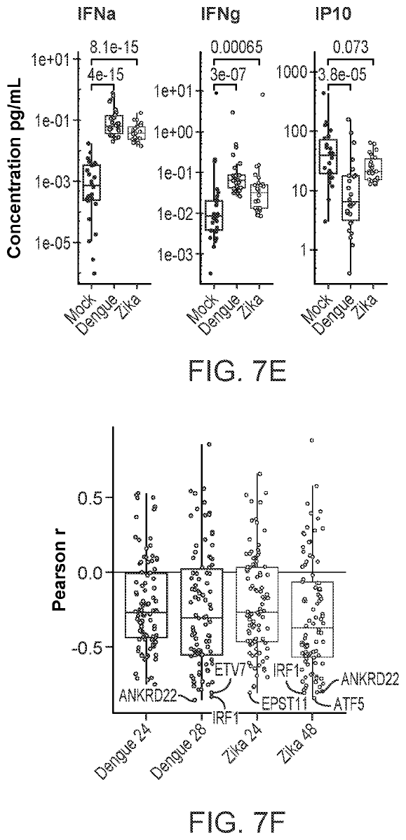

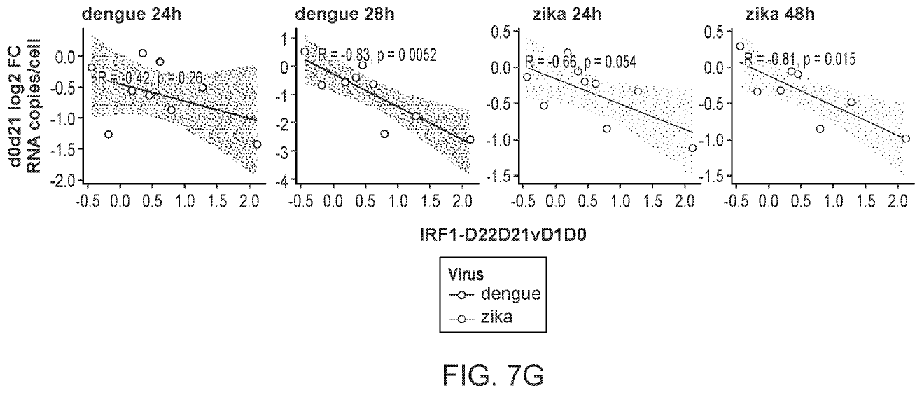

To determine if the observed epigenomic changes resulted in enhanced resistance to viral infections, we infected PBMCs at day 0, 21 and 42 with Dengue or Zika virus (FIG. 7a). After infection, we cultured cells for 0, 24, and 48 h and determined the viral copy number using qPCR (FIG. 7b). We observed increased numbers of Zika and Dengue virus copies at 24 h and reduction at 48 h following the expected cycle of infection, replication, and eventually death of the host cells (FIG. 7c). Next, we compared the viral titers at day 21 and 42 after vaccination with the pre-vaccination titers at day 0 for each subject. Strikingly, we observed a significant reduction in viral titers for both, Dengue and Zika virus, at day 21 after vaccination (FIG. 7d). Importantly, in many subjects, we observed reduced viral titers as late as 42 days after initial vaccination (FIG. 7d). Next, we determined the cytokine production of the infected PBMC cultures at 24 h after infection using ELISA (FIG. 7e). While Dengue and Zika virus induced the production of both IFNa and IFNg, we observed that Dengue virus suppressed the production of IP10 (FIG. 7e). Finally, we correlated the change in viral titers at d0 compared to d21 with the change in vaccine-induced expression of antiviral genes that were associated with open chromatin (FIG. 6j red quadrant). The majority of these genes correlated negatively with viral titers (FIG. 7f). Strikingly, IRF1 was amongst the top genes negatively correlating (r<−0.8) with both Dengue and Zika titers (FIG. 7f) and subjects with enhanced IRF1 expression at day 21 showed reduced viral titers at the same time point (FIG. 7g). In addition, the antiviral gene ANKRD22, which is involved in immunity to both Dengue and Chikungunya infection (Soares-Schanoski et al., 2019), was highly negatively correlated with Zika and Dengue titers, too.

Together, these data demonstrate that, despite the presence of AP-1-based suppression, AS03 induces an epigenomic state of enhanced antiviral immunity that enables increased production of interferons and enhanced control of heterologous viral infection.

Here, we used several bulk- and single-cell approaches to construct the single-cell epigenomic landscape of immunity to three distinct influenza vaccines in humans. Our results demonstrate that two vaccines, the seasonal influenza vaccine TIV and the pandemic influenza vaccine H5N1+AS03, induce profound and persistent global epigenomic changes in the peripheral immune system, especially in the myeloid compartment, and that these epigenomic changes alter the functionality of immune cells upon re-stimulation in-vitro and in-vivo. The observed changes were most pronounced at three to four weeks after vaccination but traces of an altered epigenomic landscape were still detected as late as 180 days after initial vaccination. Notably, single-cell analysis revealed that these alterations are driven by previously unappreciated epigenomic substrates within the monocyte population.

Based on their molecular and functional characteristics, the observed epigenomic changes can be broadly classified into two distinct types: 1) a state of innate immune refractoriness that is characterized by reduced histone acetylation, reduced PADI4 levels, reduced AP-1 accessibility and diminished production of innate cytokines; 2) a state of heightened antiviral vigilance defined by increased IRF accessibility, elevated antiviral gene expression, increased interferon production, and, most importantly, enhanced control of heterologous viral infections. Importantly, both states occur simultaneously and in the same single cell. While seemingly paradoxical, this superimposition might represent an evolutionary adaptation to avoid excess inflammatory host damage during late stages of infections, while maintaining a state of immunological vigilance against viral infections.

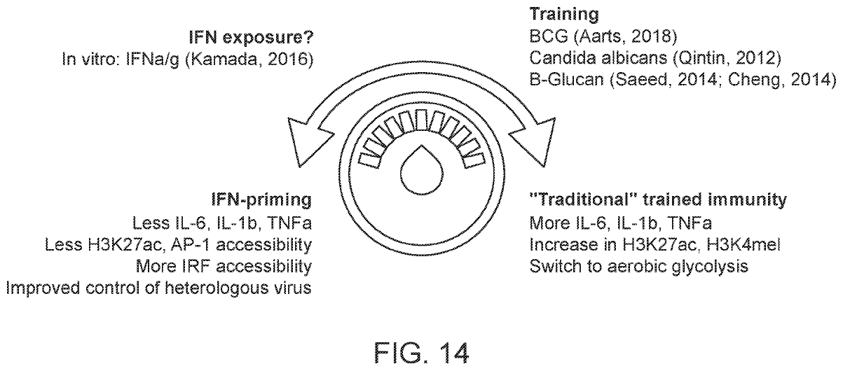

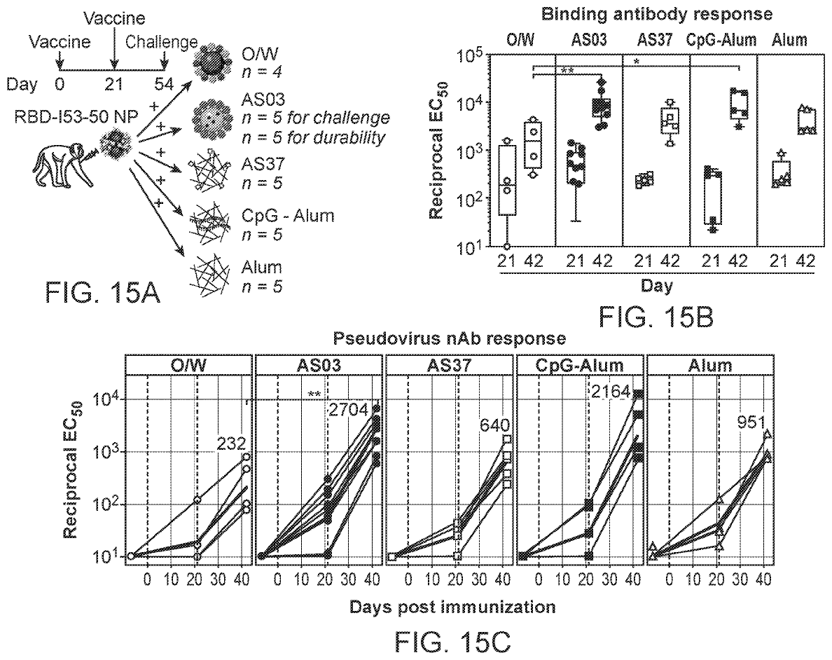

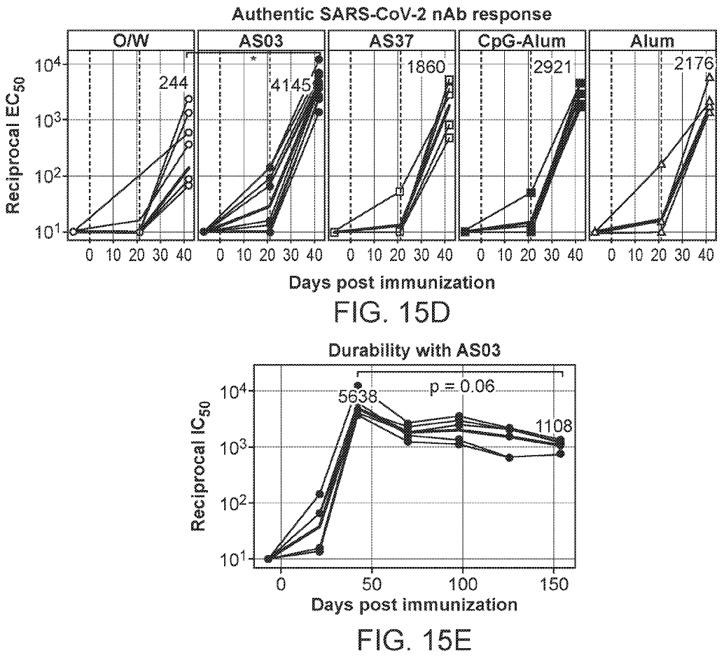

Our findings were unexpected as researchers previously observed that the live-attenuated BCG vaccine against tuberculosis induces elevated H3K27ac levels in CD14⁺ monocytes which coincided with enhanced cytokine production in these cells. Our results, in contrast, suggest that vaccine-induced epigenomic reprogramming of immune cells is more complex. Given the observed reduction in H3K27ac levels in association with immune refractoriness in this study, the possibility arises that histone acetylation could represent a bi-directional regulator, powered by epigenomic substrates at the single-cell level, that can be raised or lowered to manipulate monocyte cytokine production accordingly, akin to a thermostat dial (FIG. 14). In addition, our data demonstrate that multiple distinct epigenomic states, such as antiviral vigilance and immune refractoriness, can be superimposed within the same cell. This suggests a nuanced and compartmentalized reprogramming process that allows the independent adjustment of disparate chromatin loci to mediate distinct immunological processes in parallel. Importantly, this superimposition is encoded at the single-cell level as single monocytes and dendritic cells displayed elevated IRF and diminished AP-1 accessibility at the same time.

Single-cell analysis further revealed multiple clusters within the classical monocytes population based on differences in chromatin accessibility. Notably, all of these epigenomic subclusters existed before vaccination and their abundance within the pool of circulating cells shifted during the course of vaccination driving the observed bulk level changes. The transcription factor families underlying the observed heterogeneity, AP-1 and CEBP, were previously described as key players in monocyte-to-macrophage differentiation and classical-to-non classical monocyte differentiation, respectively. AP-1 is also a central regulator of inflammation and our Hotspot analysis revealed differences in accessibility at inflammatory loci between epigenomic subclusters. This might suggest that distinct functional and ontogenetic fates could be imprinted within the epigenome of single monocytes. Indeed, it was recently hypothesized that classical monocytes could represent a heterologous population of cells, some pre-committed to tissue infiltration and macrophage differentiation and others primed for differentiation into non-classical monocytes.

Our systems biology analysis also extends the current CD14⁺ monocyte-focused understanding of vaccine-induced epigenomic reprogramming with insights into other circulating cells of the innate immune system. Previously, it was not known whether CD14⁻ monocytes or dendritic cells would exhibit epigenomic changes after vaccination in humans. Here, we show that influenza vaccination induces lasting epigenomic changes also in mDCs which shared many of the molecular characteristics with classical monocytes. In contrast, non-classical CD14⁻CD16⁺ monocytes and pDCs presented less pronounced and more short-lived alterations in their epigenomic state.

With respect to the molecular mechanisms driving the epigenomic changes, we observed that the state of antiviral vigilance was associated with enhanced IRF1 and STAT1 activity. It is established that IRF and STAT signaling promotes antiviral immunity and KO models lacking IRF1 or STAT1 are more susceptible to viral infection. In contrast, the state of immune refractoriness was associated with a global reduction in histone acetylation and chromatin accessibility. The magnitude of the observed changes suggests a comprehensive switch towards a broadly restrictive chromatin state. Our TF motif-based analysis revealed that especially AP-1 loci are affected by this process. AP-1 is a dimeric TF composed of different members of the FOS, JUN, ATF, and JDP families and our gene expression analysis suggests that multiple members including FOS, JUN, JUNB, and ATF3 are involved. While the role as of AP-1 a key regulator of differentiation, inflammation and polarization in myeloid cells is well described, recent research position it as a central epigenomic regulator, too.

From a biological perspective, we made multiple observations that show a direct link between the epigenomic state and immune protection. Our results from the H5N1+AS03 vaccine revealed that PBMCs from vaccinated individuals control infection with the heterologous Dengue and Zika virus more efficiently than pre-vaccination PBMCs. These results, in combination with the enhanced expression of antiviral genes and increased levels of IP-10 and IFN gamma production in-vivo, show that the epigenomic state of antiviral vigilance can provide non-specific protection against viral infections unrelated to the vaccine virus. In contrast, TIV induced a profound state of immune refractoriness at four weeks after vaccination. This suggests that vaccination with TIV, in contrast to H5N1+AS03, could potentially increase the susceptibility to infections late after vaccination. It is important to highlight that there is ample evidence that TIV does prevent influenza and our own study found induction of robust anti-influenza antibody titers. Given the observed immune refractoriness, it could be beneficial to administer TIV together with an adjuvant, such as AS03. This adjuvanted TIV would overcome the induced immune refractoriness with an epigenomics-driven state of antiviral vigilance. Indeed, a phase 3 clinical trial comparing the response to TIV vs TIV+AS03 in more than 43,000 Elderly individuals demonstrated that TIV+AS03 led to a profound reduction in all-cause death and pneumonia compared to TIV alone while influenza-specific immunity was only somewhat increased. While subsequent placebo-controlled, double-blinded studies are needed to definitely proof the clinical benefit, our results demonstrate a previously unknown mechanism of action for adjuvants to provide non-specific protection via epigenomic reprogramming.

In conclusion, we investigated how vaccination with three distinct influenza vaccines alters the epigenomic immune cell landscape at the single-cell level. Our results demonstrate the ability of influenza vaccines to induce lasting epigenomic changes that drive altered functional immune cell profiles at the single-cell level and enable heterologous protection against non-vaccine viruses. Our findings have important implications for the design of future vaccines and provide for the development of “epigenomic” adjuvants that provide broadly specific protection by manipulating the epigenomic landscape.

### Experimental Subject Details

TABLE 1
Subject and vaccine information
Cohorts
TIV

| | Participants | Median Age | | |
|---|---|---|---|---|
| Treatment | (no.) | (range) | Gender (%) | Race (%) |
| Antibiotics | 10 | 29 | Male (70%) | White (50%) |
| | | (24-38) | Female (30%) | Black or African American (30%) |
| | | | | Other (20%) |
| Control | 11 | 27 | Male (64%) | White (63%) |
| | | (24-35) | Female (36%) | Black or African American (27%) |
| | | | | Other (10%) |

H5N1/H5N1 + AS03

| | Participants | Median Age | | |
|---|---|---|---|---|
| Vaccine | (no.) | (range) | Gender (%) | Race (%) |
| H5N1 | 16 | 26 | Male (44%) | White (44%) |
| | | (22-40) | Female (56%) | Black (25%) |
| | | | | Asian (19%) |
| | | | | Other (12%) |
| H5N1 + AS03 | 34 | 28 | Male (38%) | White (77%) |
| | | (21-44) | Female (62%) | Black (18%) |
| | | | | Asian (3%) |
| | | | | Other (2%) |

### Vaccines

TIV

| | Vaccine | | | |
|---|---|---|---|---|
| Vaccine | Brand/Season | H1N1 strain | H3N2 strain | B strain |
| TIV | Fluzone | A/California/ | A/Texas/ | B/Masschusetts/ |
| | 2014-2015 | 07/2009 | 50/2012 | 02/2012 |

TIV. The study design was as described in phase 1 of the original publication (Hagan et al., 2019) and the study was conducted in Atlanta, GA. In brief, during the 2014-2015 seasons, we enrolled a total of 21 healthy adults who were randomized into antibiotics-treated (n=10) and control (n=11) groups. Subjects were males and non-pregnant females between the ages of 18-40 who met the eligibility criteria as listed on clinicaltrials.gov (NCT02154061). Subject demographics are listed in. The antibiotics treatment consisted of a cocktail of neomycin, vancomycin, and metronidazole, all given orally, for five days. Antibiotic treatment started 3 days before the day of vaccination and continued until one day after for the antibiotics-treated group. All the study participants were vaccinated with Fluzone for the 2014-2015 season. Written informed consent was obtained from each subject and protocols were approved by Institutional Review Boards of Emory University.

H5N1/H5N1+AS03. This study was conducted in Atlanta, GA. Subjects were males and non-pregnant females who met the eligibility criteria as listed on clinicaltrials.gov (NCT01910519). We enrolled a total of 50 healthy adults who were randomized into two groups receiving either the adjuvanted (H5N1+AS03, n=34) or unadjuvanted (H5N1, n=16) GSK avian influenza vaccine. Subject demographics are listed in. Written informed consent was obtained from each subject and protocols were approved by Institutional Review Boards of Emory University.

In-vitro stimulation and intracellular flow cytometry experiments. Samples from healthy subjects were collected at Stanford Blood Center or derived from the before-vaccination time point of a previous vaccination trial (Nakaya et al., 2015). All subjects provided a confidential medical history card and completed informed consent to donate blood for clinical or research uses. We exclude subjects with known diseases, including but not limited to HIV, and hepatitis infections. Purification of buffy coat or LRS chamber from whole blood was performed at Stanford Blood Center to enrich for leukocytes prior to PBMC isolation.

From the vaccination trial (Nakaya et al., 2015), only samples from subjects aged 26-41 were selected for this paper. Samples were only selected from the before vaccination time point at day 0. Written informed consent was obtained from each subject with institutional review and approval from the Emory University Institutional Review Board.

### Method Details

Cells, plasma and RNA isolation. Peripheral blood mononuclear cells (PBMCs) and plasma were isolated from fresh blood (CPTs; Vacutainer with Sodium Citrate; BD), following the manufacturer's protocol. For samples from Stanford Blood Center, PBMCs isolated from whole blood, buffy coat or LRS chamber by Ficoll density gradient centrifugation using Ficoll-Paque PLUS (GE Healthcare, #17-1440-02). PBMCs were frozen in DMSO with 10% FBS and stored at −80 C and then transferred on the next day to liquid nitrogen freezers (−196 C). Plasma samples from CPTs were stored at −80 C. Trizol (Invitrogen) was used to lyse fresh PBMCs (1 mL of Trizol to −1.5×10\hat{ }6 cells) and to protect RNA from degradation. Trizol samples were stored at −80 C.

Mass cytometry sample processing, staining, barcoding and data acquisition. Cryopreserved PBMCs were thawed and incubated in RPMI 1640 media (ThermoFisher) containing 10% FBS (ATCC) at 37° C. for 1 hour prior to processing. Cisplatin (ENZO Life Sciences) was added to 10 μM final concentration for viability staining for 5 minutes before quenching with CyTOF Buffer (PBS (ThermoFisher) with 1% BSA (Sigma), 2 mM EDTA (Fisher), 0.05% sodium azide). Cells were centrifuged at 400 g for 8 minutes and stained with lanthanide-labeled antibodies against immunophenotypic markers in CyTOF buffer containing Fc receptor blocker (BioLegend) for 30 minutes at room temperature (RT). Following extracellular marker staining, cells were washed 3 times with CyTOF buffer and fixed in 1.6% PFA (Electron Microscopy Sciences) at 1×106 cells/ml for 15 minutes at RT. Cells were centrifuged at 600 g for 5 minutes post-fixation and permeabilized with 1 ml ice-cold methanol (Fisher Scientific) for 20 minutes at 4° C. 4 ml of CyTOF buffer was added to stop permeabilization followed by 2 PBS washes. Mass-tag sample barcoding was performed following the manufacturer's protocol (Fluidigm). Individual samples were then combined and stained with intracellular antibodies in CyTOF buffer containing Fc receptor blocker (BioLegend) overnight at 4° C. The following day, cells were washed twice in CyTOF buffer and stained with 250 nM 191/1931r DNA intercalator (Fluidigm) in PBS with 1.6% PFA for 30 minutes at RT. Cells were washed twice with CyTOF buffer and once with double-deionized water (ddH2O) (ThermoFisher) followed by filtering through 35 μm strainer to remove

Bulk stimulation experiment. Aliquots of thawed PBMCs from the EpiTOF experiment described above were washed and resuspended in RPMI 1640 (Corning, 10-040-CV) containing 10% FBS (Corning, 35-011-CV) and 1× Antibiotics/Antimycotics (Lonza, 17-602E) [complete media abx] at 4×10\hat{ }6 cells/mL. 100 μL of cell solution were added to each well of a 96-well round-bottomed tissue culture plate and mixed with 100 μL of either complete media abx (unstim), a cocktail of synthetic TLR ligands mimicking bacterial pathogens (bac: 0.025 μg/mL LPS, 0.3 μg/mL Flagellin, 10 μg/mL Pam3CSK4), or a cocktail of synthetic TLR ligands mimicking viral pathogens (vir: 4 μg/mL R848, 25 μg/mL pl:C). Depending on cell numbers, PBMCs from each sample were stimulated with all 3 conditions in duplicate. After 24 h of incubation at 37 C and 5% CO₂, cells were spun down, supernatant was carefully transferred into new plates, and immediately frozen at −80 C until further analysis using Luminex.

Luminex TIV. The Luminex assay was performed by the Human Immune Monitoring Center, Stanford University School of Medicine. Human 62-plex custom Procarta Plex Kits (Thermo Fisher Scientific) were used according to the manufacturer's recommendations with modifications as follows: Briefly, Antibody-linked magnetic microbeads were added to a 96-well plate along with custom Assay Control microbeads (Assay Chex) by Radix Biosolutions. The plates were washed in a BioTek ELx405 magnetic washer (BioTek Instruments). Neat Cell culture supernatants (25 ul) and assay buffer (25 ul) were added to the 96 well plate containing the Antibody-coupled magnetic microbeads, and incubated at room temperature for 1 h, followed by overnight incubation at 4° C. Room temperature and 4° C. incubation steps were performed on an orbital shaker at 500-600 rpm. Following the overnight incubation, plates were washed in a BioTek ELx405 washer (BioTek Instruments) and then kit-supplied biotinylated detection Ab mix was added and incubated for 60 min at room temperature. Each plate was washed as above, and kit-supplied streptavidin-PE was added. After incubation for 30 min at room temperature, wash was performed as described, and kit Reading Buffer was added to the wells. Each sample was measured in two technical replicates where cell numbers allowed. Plates were read using a FlexMap 3D Instrument (Luminex Corporation). Wells with a bead count<50 were flagged, and data with a bead count<20 were excluded.

Luminex H5N1/H5N1+AS03. This assay was performed by the Human Immune Monitoring Center at Stanford University. A custom 41 plex from EMD Millipore kits was assembled and included: 1. A Pre-mixed 38 plex Milliplex Human Cytokine/Chemokine kit (CAT#HCYTMAG-60K-PX38) 2. ENA78/CXCL5 (CAT #HCYP2MAG-62K-01) 3. IL-22 (CAT#HTH17MAG-14K-01). 4. IL-18 (HIL18MAG-66K). Manufacturer's recommendations were followed with modifications described. Briefly: neat supernatant samples (25 ul) were mixed with antibody-linked magnetic beads in a 96-well plate containing assay buffer, for an overnight incubation at 4° C. Cold and Room temperature incubation steps were performed on an orbital shaker at 500-600 rpm. Plates were washed twice with wash buffer in a BioTek ELx405 washer (BioTek Instruments). Following one-hour incubation at room temperature with biotinylated detection antibody, streptavidin-PE was added for 30 minutes. Plates were washed as above, and PBS was added to wells for reading in the Luminex FlexMap3D Instrument with a lower bound of 50 beads per sample per cytokine. Each sample was measured in duplicate wells where cell numbers allowed. Custom Assay Chex control beads were added to all well (Radix Biosolutions). Wells with a bead count<50 were flagged, and data with a bead count<20 were excluded.

H3K27ac antibody conjugation. α-H3K27ac antibody was labeled using the Lightning-Link Rapid DyLight 488 Antibody Labeling Kit according to manufacturer's instructions (Novus Biologicals, 322-0010). In brief, 100 μg of antibody was mixed with 10 μL of LL-Rapid modifier reagent and added onto the lyophilized dye. After mixing, solution was incubated at room temperature overnight in the dark. The next morning, 10 μL of LL-Rapid quencher reagent was added.

In-vitro stimulation and intracellular flow cytometry experiments. Cryopreserved PBMCs were thawed, counted, and resuspended in RPMI 1640 (Corning, 10-040-CV) supplemented with 10% FBS (Corning, 35-011-CV) [complete media] at a concentration of 4×10\hat{ }6 cells/mL. Next, 150 μL of cell suspension (6×10\hat{ }5 cells) was added to each well of a 96-well round-bottomed tissue culture plate and mixed with 50 μL of inhibitor solution containing either Trichostatin A (TSA; CST, 9950S), A-485 (Tocris, 6387), or CI-Amidine (EMD Millipore, 506282) in complete media. After 2 h of incubation at 37 C and 5% CO₂, the cells were stimulated by adding either LPS (0.025 μg/mL; Invivogen, tlrl-pb5lps) or R848 (4 μg/mL; Enzo Life Sciences, ALX-420-038-M005) to the cultures. After another 2 h of incubation, Brefeldin A (10 μg/mL; Sigma Aldrich, B7651-5MG) was added to all cultures and cells were incubated for a final 4 h. After a total of 8 h of incubation, cells were washed twice with 150 μL PBS (GE Life Sciences, SH30256.LS) and stained for viability using 100 μL of Zombie UV Fixable Viability Dye in PBS (1:1000; Biolegend, 423108). After incubating for 30 minutes at 4 C in the dark, cells were washed twice with 1504 PBS and blocked with 100 μL of PBS supplemented with 5% FBS, EDTA (2 mM; Corning, 46-034-c1), and human IgG (5 mg/mL; Sigma Aldrich, G4386-5G) [blocking buffer] for 15 minutes at 4 C in the dark. After incubation, cells were stained for surface markers with 100 μL of antibody cocktail containing α-CD14 BUV805, α-CD3, CD19, CD20 BUV737, α-CD123 BUV395, α-HLA-DR BV785, α-CD16 BV605, α-CD56 PE-CY7, α-CD11c APC-eFluor780 in blocking buffer for 20 minutes at 4 C in the dark. Next, cells were washed twice with 150 μL PBS, and fixed in 200 μL eBioscience Foxp3 Fixation/Permeabilization solution (ThermoFisher Scientific, 00-5523-00) for 30 minutes at 4 C in the dark. Afterwards, cells were washed twice with 100 μL eBioscience Foxp3 permeabilization buffer and blocked with 100 μL permeabilization buffer containing human IgG (5 mg/mL) overnight at 4 C in the dark. Cells were washed and stained for intracellular markers with 25 μL of antibody cocktail containing α-IL-1b Pacific Blue, α-H3K27ac DyLight 488, α-TNFa PE-Dazzle, α-p-c-Jun PE, and α-H3 AF647 in permeabilization buffer containing human IgG (5 mg/mL) for 60 minutes at 4 C in the dark. Finally, cells were washed twice with 150 μL of permeabilization buffer, resuspended in 100 μL PBS containing 0.5% FBS and 2 mM EDTA [FACS buffer], and acquired using a BD FACSymphony flow cytometer. Data was analyzed using Flowjo X software (BD). Briefly, cells were identified via FSC/SSC, doublets were discarded via SSC-A/SSC-H and FSC-A/FSC-H gates, and dead cells were discarded as Zombie UV Fixable Viability Dye high. Monocytes were then identified as CD3⁻CD19⁻CD20⁻ and CD14⁺.

FACS sorting—bulk ATAC-seq/RNA-seq. Cryopreserved PBMCs were thawed, washed, counted, and resuspended in PBS (GE Life Sciences, SH30256.LS). 5-10×10⁶ cells were washed once more with 2 mL of PBS and stained for viability using 500 μL of Zombie UV Fixable Viability Dye in PBS (1:1000; Biolegend, 423108). After incubating for 30 minutes at 4 C in the dark, cells were washed with 2 mL of PBS and resuspended in 500 μL blocking buffer. After spinning cells down, supernatant was discarded, and cells were resuspended in 50 μL antibody cocktail containing α-CD3, CD19, CD20 BUV737, α-CD123 BUV395, α-HLA-DR BV785, α-CD14 BV605, α-CD56 BV510, α-CD1c BV421, α-CD327 AF488, α-CD370 PE, α-CD11c APC-eFluor780, α-CD15 AF700, and α-Axl APC in blocking buffer. Cells were stained for 15 minutes at 4 C in the dark. Finally, cells were washed with 2 mL of FACS buffer, resuspended in PBS containing 5% FBS at 10-20×10\hat{ }6 cells/mL, and stored at 4 C before sorting on a FACS Aria Fusion (BD). During sort, live innate cells were identified by gating on Viability Dye⁻CD3⁻CD19⁻CD20⁻ cells. Within this population, CD14⁺ monocytes were identified as CD14⁺, mDCs were identified as CD14⁻CD56⁻HLA-DR⁺CD16⁻CD11c⁺CD123⁻, and pDCs were identified as CD14⁻CD56⁻HLA-DR⁺CD16⁻CD11c⁻CD123⁺.

Omni ATAC-seq of purified immune cells. Atac was performed on purified innate immune cell subsets immediately after sorting based on the low-input Omni-Atac protocol described before (Corces et al., 2017). In brief, 1,500-5,500 cells were washed with ATAC resuspension buffer (10 mM Tris-HCl pH 7.5 [Invitrogen, 15567027], 10 mM NaCl [Invitrogen, AM9760G], 3 mM MgCl₂ [Invitrogen, AM9530G], in water [Invitrogen, 10977015]) and supernatant was carefully aspirated, first using a P1000, then a P200 pipette. Next, 10 μL transposition mix (0.5 μL Tn5, 0.1 μL 10% Tween-20, 0.1 μL 1% Digitonin, 3.34 PBS, 14 water, and 5 μL tagmentation buffer) was added to the pellet and cells were resuspended by pipetting up and down 6 times. Tagmentation buffer was prepared locally by resuspending 20 mM Tris-HCl pH 7.5, 10 mM MgCl₂, and 20% Dimethyl Formamide (Sigma Aldrich, D4551-250ML) in water. Cells were incubated at 37 C for 30 minutes under constant mixing. After tagmentation, the reaction was cleaned up using the MinElute PCR Purification Kit (Qiagen, 28006) according to manufacturer's instructions. Cleaned DNA was eluted in 21 μL of elution buffer, stored at −20 C, and shipped to Active Motif for sequencing library preparation. At Active Motif, tagmented DNA was amplified with 10 cycles of PCR using customized Nextera PCR Primers 1 and 2 (see Key Resource table), and purified using Agencourt AMPure SPRI beads (Beckman Coulter, A63882). Resulting material was quantified using the KAPA Library Quantification Kit for Illumina platforms (Roche, 07960255001), and sequenced with PE42 sequencing on the NextSeq 500 sequencer (Illumina).

Bulk RNA-seq of purified immune cells. Bulk RNA-seq was performed on purified CD14⁺ monocytes after sorting. In brief, after sorting, 5,500 cells were washed, resuspended in 350 μL chilled Buffer RLT (Qiagen, 79216) supplemented with 1% beta-Mercaptoethanol (Sigma, M3148-25ML), vortexed for 1 minute, and immediately frozen at −80 C. RNA was isolated using the RNeasy Micro kit (Qiagen, 74004) with on-column DNase digestion. RNA quality was assessed using an Agilent Bioanalyzer and total RNA was used as input for cDNA synthesis using the Clontech SMART-Seq v4 Ultra Low Input RNA kit (Takara Bio, 634894) according to the manufacturer's instructions. Amplified cDNA was fragmented and appended with dual-indexed bar codes using the NexteraXT DNA Library Preparation kit (Illumina, FC-131-1096). Libraries were validated by capillary electrophoresis on an Agilent 4200 TapeStation, pooled at equimolar concentrations, and sequenced on an Illumina NovaSeq6000 at 100SR, yielding 20-25 million reads per sample.

FACS sorting—scATAC-seq/RNA-seq. Cryopreserved PBMCs were thawed and innate immune cell subsets were isolated using FACS as described above (FACS sorting—bulk ATAC-seq/RNA-seq). Within the live gated cells, CD14⁺ monocytes were identified as CD14⁺ (fraction A) while a mixture of the remaining monocyte and dendritic cell subsets was identified as CD14⁻CD56⁻HLA-DR⁺ (fraction B). After sorting and depending on the number of isolated cells, fraction A and B were mixed at a 2:1 ratio to yield a solution of monocytes and dendritic cells enriched for CD14⁻ cells.

scRNA-seq. FACS-purified cells were resuspended in PBS supplemented with 1% BSA (Miltenyi), and 0.5 U/μL RNase Inhibitor (Sigma Aldrich). About 9,000 cells were targeted for each experiment. Cells were mixed with the reverse transcription mix and subjected to partitioning along with the Chromium gel-beads using the 10× Chromium system to generate the Gel-Bead in Emulsions (GEMs) using the 3′ V3 chemistry (10× Genomics). The RT reaction was conducted in the C1000 touch PCR instrument (BioRad). Barcoded cDNA was extracted from the GEMs by Post-GEM RT-cleanup and amplified for 12 cycles. Amplified cDNA was subjected to 0.6×SPRI beads cleanup (Beckman, B23318). 25% of the amplified cDNA was subjected to enzymatic fragmentation, end-repair, A tailing, adapter ligation and 10× specific sample indexing as per manufacturer's protocol. Libraries were quantified using Bioanalyzer (Agilent) analysis. Libraries were pooled and sequenced on an NovaSeq 6000 instrument (Illumina) using the recommended sequencing read lengths of 28 bp (Read 1), 8 bp (i7 Index Read), and 91 bp (Read 2).

scATAC-seq. FACS-purified cells were processed for single nuclei ATAC-seq according to the manufacturer's instructions (10× Genomics, CG000168 Rev D). Briefly, nuclei were obtained by incubating PBMCs for 3.20 minutes in freshly prepared Lysis buffer following manufacturer's instructions for Low Cell Input Nuclei Isolation (10× Genomics, CG000169 Rev C). Nuclei were washed and resuspended in chilled diluted nuclei buffer (10× Genomics, 2000153). Next, nuclei were subjected to transposition for 1 h at 37 C on the 01000 touch PCR instrument (BioRad) prior to single nucleus capture on the 10× Chromium instrument. Samples were subjected to post GEM cleanup, sample index PCR, cleanup and library QC prior to sequencing according to the protocol. Samples were pooled, quantified and sequenced on NovaSeq 6000 instrument (Illumina) with at least minimum recommended read depth (25000 read pairs/nucleus).

IFNα SIMOA. Frozen plasma was shipped to Qunaterix and analyzed using the Simoa® IFN-α Advantage Kit (Quanterix, 100860) according to manufacturer's instructions. In brief, plasma and reagents were thawed at room temperature. Cailbrators, controls, and plasma were transferred to assay plates. Beads were vortexed for 30 seconds and prepared reagents and samples were loaded into a HD-1/HD-X instrument and analyzed with standard settings. All samples were run in duplicate.

Detection of IFNα and IFNγ in plasma and cell culture supernatants. Frozen plasma or supernatant was thawed at room temperature and analyzed using the IFNα and IFNγ Human ProQuantum Immunoassay Kits according to manufacturer's instructions. In brief, samples were mixed with assay dilution buffer at a 1:5 or 1:2 ratio and protein standard was serially diluted in assay dilution buffer. Next, Antibody-conjugates A and B were mixed with Antibody-conjugate dilution buffer and added to each well of a 96-well qPCR plate (Bio-Rad, #HSP9601). Next, diluted sample or standard were added to each well and mixtures were incubated for 1 h at room temperature in the dark. Finally, Master max and Ligase were mixed and added to each well. QPCR was conducted on a CFX96 Touch Real-Time Detection System (Biorad) using the recommended instrument settings. After measurements were completed, CT values were calculated using a regression model and exported to the ProQuantum Cloud app that accompanied the kit (apps.thermofisher.com/apps/proquantum). ProQuantum Cloud app was then used to construct a standard curve and calculate protein concentrations from CT values.

IP-10 plasma Luminex. Plasma biomarker concentrations were assayed using a 10-analyte multiplex bead array (fractalkine, IL-12P40, IL-13, IL-1 RA, IL-1b, IL-6, IP-10, MCP-1, MIP-1α, INFβ; Millipore) prepared according to the manufacturer's recommended protocol and read using a Bio-Plex 200 suspension array reader (Bio-Rad). Data were analyzed using Bio-Plex manager software (Bio-Rad).

Viral infection assay. Dengue virus (DENY-2, Strain Thailand/16681/84) and Zika virus (PRVABC59) were propagated and titrated on Vero cells and stored at −80 C until infection. Cryopreserved human PBMCs were thawed, washed, counted, and resuspended in RPMI 1640 (Thermo Fisher, 72400-047) supplemented with 10% FBS (Corning, 35-011-CV), 1 mM Sodium pyruvate (Lonza, 13-115E), and 1× Penicillin/Streptomycin (Lonza, 17-602E) at 1.5×10\hat{ }6 cells/mL. 200 μL of cell solution (3×10\hat{ }5 cells) was added to each well of a 96-well round-bottomed tissue culture plate and cells were rested in plates for 4 h at 37 C and 5% CO₂. After resting, PBMCs were infected with DENV-2 or ZIKV at MOI 1. At 0 h, 24 h, 48 h post infection, PBMCs and supernatant were collected for RNA purification and cytokine analysis, respectively. Supernatants were immediately frozen at −20 C and stored until analysis. Cells were suspended in RNA lysis buffer and kept at −20 C until analysis. RNA was purified using the Purelink RNA kit according to manufacturer recommendations (Thermo Fisher Scientific, #12183052). For viral load detection, quantitative reverse transcription PCR (qRT-PCR) was conducted using Luna universal probe one-step RT-PCR kit (NEB, #E3006) on a CFX96 C1000 Touch Real-Time Detection System with 96-well plates (Bio-Rad, #HSP9601). RNA standards (ATCC, #VR-3229SD, VR-1843DQ) were used to generate standard curves. Viral RNA copies were normalized by cell number. Utilized primers and probes are listed in the Key Resources table.

Detection of IP-10 in culture supernatant. Culture supernatants were thawed at room temperature and analyzed using the IP-10 enzyme-linked immunosorbent assay (R&D Systems, DIP100) according to the manufacturer's instructions. In brief, samples were thawed at room temperature and mixed with assay dilution buffer at 1:2 ratio. Protein standard was serially diluted in assay dilution buffer. Samples and standards were incubated in plate for 2 h at room temperature. Plate were washed and then incubated with human IP-10 conjugate for 2 h at room temperature. After wash, substrate solution was added for 30 min. Finally, stop solution was added, A450 and A595 were read on a plate reader (Bio-Rad, iMARK). The concentration of IP-10 was determined by the number of A450-A595 based on the standard curve.

### Quantification and Statistical Analysis

Immune Cell Population Definitions and EpiTOF Data Pre-Processing. Raw data were pre-processed using FlowJo (FlowJo, LLC) to identify cell events from individual samples by palladium-based mass tags, and to segregate specific immune cell populations by immunophenotypic markers. (TIV & H5N1/H5N1+AS03). Single-cell data for various immune cell subtypes from individual subjects were exported from FlowJo for downstream computational analyses.

EpiTOF analysis. The exported Flowjo data were then normalized following the approach described in (Cheung et al., 2018). In brief, the value of each histone mark was regressed against the total amount of histones, represented by measured values of H3 and H4. For sample level analyses, the values of each histone mark were averaged for each cell type in each sample. Distances of HSC from lymphoid and myeloid epigenetic profiles were obtained by first computing centers of the epigenetic profiles for the two lineages, and then computing Euclidean distances from the centers for each individual HSC. Distances of HSC from epigenetic profiles of specific cell types were similarly obtained by computing Euclidean distances from the centers of the epigenetic profiles for each cell type. Statistical significance of the differences between groups at the sample level was assessed by computing an effect size with Hedges′g formula (Hedges and Olkin, 2014). All p-values were corrected for multiple comparisons with the Benjamini-Hochberg method. Dimensionality reduction was performed with applying UMAP. For single cell analyses, the normalized values were used as input. Correlation between variables was computed using Pearson's correlation coefficient. All the analyses were performed using the R framework for statistical computing (Version 3.6.3) (R Core Team, 2020).

TIV bulk gene expression analysis. Processed data and normalized in Bioconductor by RMA, which includes global background adjustment and quantile normalization. Samples from phase1 subjects in the antibiotics and control arm of the study were selected and statistical tests and correlation analyses were performed using MATLAB. Test details and significance cutoffs are reported in figure legends.

Luminex analysis. Statistical analysis was conducted in R (v 4.0.2) (R Core Team, 2020). First, MFI data was log 2 transformed and average MFI and CV was calculated from duplicate cultures where available. For samples with CV>0.25, the duplicate that was closer to the average of all samples of that subject was kept and the other discarded. In case no other sample was available and CV>0.25, the sample was discarded. Wells without indication of cytokine production were excluded. Statistical tests, correlation analysis, and hierarchical clustering were performed using the R packages stats (v 4.0.2), ggpubr (v 0.4.0) and pheatmap (v 1.0.12). Test details and statistical cutoffs are reported in the figure legends.

Bulk ATAC-seq pre-processing. Analysis of ATAC-seq data was very similar to the analysis of ChIP-Seq data. Reads were aligned using the BWA algorithm (mem mode; default settings; v 0.7.12). Duplicate reads were removed, only reads mapping as matched pairs and only uniquely mapped reads (mapping quality>=1) were used for further analysis. Alignments were extended in silico at their 3′-ends to a length of 200 bp and assigned to 32-nt bins along the genome. The resulting histograms (genomic “signal maps”) were stored in bigWig files. Peaks were identified using the MACS algorithm (v 2.1.0) (Zhang et al., 2008) at a cutoff of p-value 1 e-7, without control file, and with the −nomodel option. Peaks that were on the ENCODE blacklist of known false ChIP-Seq peaks were removed. Signal maps and peak locations were used as input data to Active Motif's proprietary analysis program, which creates Excel tables containing detailed information on sample comparison, peak metrics, peak locations and gene annotations. For differential analysis, reads were counted in all merged peak regions (using Subread), and the replicates for each condition were compared using DESeq2 (v 1.24.0) (Love et al., 2014).

Bulk ATAC-seq analysis. Quality control analysis of ATAC-seq data was performed using Rockefeller University workshop on analysis of ATAC-seq data in R and Bioconductor. Of 57 unique samples processed, 52 passed QC criteria and, on average, we detected more than 42,000 genomic regions and more than 15×10⁶ unique ATAC tags per sample while the average fraction of reads in peaks was larger than 35%. Passed samples showed the characteristic fragment length and TSS enrichment distribution. DARs were annotated as promoter, distal and trans regulatory peak for a particular gene based on the distance from the middle of the peak to the nearest transcription start site (TSS) using the ChlPpeakAnno package in R (v. 3.24.1). Promoter, distal and trans regulatory peaks were defined as −2000 bp to +500 bp, −10 kbp to +10 kbp—promoter, and <−10 kbp or >+10 kbp from TSS, respectively. The hypergeometric distribution-based enrichment analysis was performed to identify the significance of the DARs. Reactome pathways and TF-target relationship using Chip-seq data from ENCODE (both downloaded from https://maayanlab.cloud/chea3/) were used to identify overrepresented pathways and TFs.

EnrichmentMap Pipeline Collection (v 1.1.0) (Merico et al., 2010) for CytoScape (v 3.8.2) (Shannon et al., 2003) was used to create the pathway network. Significantly enriched Reactome pathways (p<=0.05) for each genomic region were used as input. Pathways were clustered and annotated using the AutoAnnotate function within the pipeline. To test for enrichment of TF motifs in DARs, the chromVAR (v 1.8.0) (Schep et al., 2017) and motifmatchr (v 1.8.0) packages were used in R (v 3.6.0) (R Core Team, 2020). In brief, TF motifs were downloaded from the JASPAR2016 core Homo sapiens database (Mathelier et al., 2016) and merged regions were annotated for the presence of all TF binding motifs using the matchMotifs (motifmatchr) function with standard settings. Hypergeometric distribution-based enrichment analysis was then performed to identify enrichment of TF motifs in DARs. To determine the relationship between EpiTOF and ATAC-seq data, the Pearson correlation was computed between EpiTOF H3K27ac levels and normalized read counts in each merged peak region. Positively correlated merged peak regions with p-value<=0.05 were selected for functional annotation. Enrichment analysis was performed as described above.

Bulk RNA-seq of purified immune cells. Alignment was performed using STAR version 2.7.3a (Dobin et al., 2013) and transcripts were annotated using GRCh38 Ensembl release 100. Transcript abundance estimates were calculated internal to the STAR aligner using the algorithm of htseq-count (Anders et al., 2015). DESeq2 version 1.26.0 (Love et al., 2014) was used for differential expression analysis using the Wald test with a paired design formula and using its standard library size normalization.

Analysis of bulk transcriptomics data from previous TIV studies. Processed bulk transcriptomics data from nine independent TIV studies conducted between 2007 and 2012 were obtained from GEO (accessions: GSE47353, GSE59635, GSE29619, GSE74813, GSE59654, GSE59743, GSE74811, GSE29617, GSE74816). After removing samples and genes with missing values as well as extraordinary vaccine time points, we selected only samples from subjects matching the same age range as the current study: 18-45 years of age. The remaining samples were batch corrected using ComBat from the sva package in R (v 3.36.0) with study as batch, no covariates, and otherwise standard settings. Statistical tests were performed using the R base and ComplexHeatmap (v 2.4.3) packages. Test details and statistical cutoffs are reported in the figure legends.

scATAC analysis. The CellRanger-atac pipeline (v1.1.0) by 10× Genomics was used for alignment (GRCh38 reference genome), de-duplication, and identification of cut sites for each sample. The samples were then combined using the CellRanger-atac aggregation procedure without depth normalization (--normalize=none). The resulting fragment file was read into SnapATAC (Fang et al., 2020). SnapATAC was used to bin the genome (bin size of 5K) and create a cell-by-bin count matrix. We called cells as barcodes with at latest 1000 UMIs, and a promotor ratio (defined as: (fragments in promoter regions+1)/(total fragments+1)) of at least 0.1, resulting in a total of [state total number of cells in each experiment], as stated in the results section. We removed bins that mapped to chrY, mitochondrial DNA, and bins that overlap with ENCODE blacklist regions (Amemiya et al., 2019). The remaining bins were used for dimensionality reduction using Truncated SVD with the irlba R package (Baglama et al., 2019), and the first 50 dimensions were then used for clustering. We then used MACS2 (Zhang et al., 2008) to call peaks within each cluster using recommended parameters for ATACseq data (--nomodel --shift 100 --ext 200 --qval 5e-2 -B --SPMR), and merged the resulting peaks to a single combined set. We then used SnapATAC to map the fragments to the combined peaks set and create a peak-by-cell binary matrix. In the H5N1/H5N1+AS03 dataset, we downsampled deeply-sequenced libraries to an average of 1500 fragments per barcode by randomly removing counts from these samples at a probability p=1500/(mean fragments per cell in the sample). We then repeated the dimensionality reduction and clustering procedure on the peak-by-cell matrix. ChromVAR (Schep et al., 2017) was used with default parameters and the JASPAR2016 (Mathelier et al., 2016) motif database to calculate motif accessibility scores and compute differentially accessible motifs in the data. Hotspot was used to identify informative gene modules that explain heterogeneity within the monocyte population (DeTomaso and Yosef, 2020). Differentially accessible regions were identified using logistic regression with the glm function in R with the design: y˜timepoint+donor+log_fragments to control for donor and library size effects. The coefficient corresponding to the time point was then used as the log FC value, and a Wald test was computed to get p-values. For numerical stability, we only included peaks that were detected in at least 5% of the cells included in each comparison. All custom scripts for preprocessing, correlation analysis, and differential accessibility analysis are posted in zenodo. The hypergeometric distribution-based enrichment analysis was performed to identify the significance of the DARs (p<=0.05 and detected in at least 5% of cells). Reactome pathways database (both downloaded from https://maayanlab.cloud/chea3/) were used to identify overrepresented pathways. Enrichr (Kuleshov et al., 2016) was used to conduct enrichment analysis of genomic regions within Hotspot modules 2, 3. Enrichr was also used to conduct enrichment analysis of DARs containing an IRF1 motif. Briefly, significant DARs (p<=0.05 and detected in at least 5% of cells) carrying an IRF1 motif, as determined by chromVAR, were selected. Next, gene names with multiple associated DARs were collapsed in case all DARs changed in the same direction or otherwise discared. Subsequently, gene list was submitted to Enrichr for enrichment using the Reactome_2016 database. Similarly, we used Enrichr together with the ChEA_2016 databases to identify TF target genes enriched in genes that were enhanced after booster vaccination with H5N1+AS03 and that overlapped with changes in accessibility at promoter regions.

scRNA analysis. The CellRanger pipeline (v3.1.0) by 10× Genomics was used for alignment (GRCh38 reference genome), demultiplexing, cell-calling, and filtering. The filtered count matrices from each sample were then aggregated using the CellRanger aggregation procedure without depth normalization (--normalize=none). The resulting count matrix was analyzed with scVI (scvi-tools v0.7.1)(Lopez et al., 2018) with default hyperparameters to fit a low-dimensional latent space, using the experiment annotation for each sample as a batch label for batch correction. Visualization, clustering, and exploratory analyses were performed with VISION (v2.1.0). Differential expression analysis between time points was performed with edgeR as described in the package documentation, using the exactTest hypothesis testing for each pairwise analysis.

Bulk transcriptomics vax010. Initial data quality was assessed by background level, 3′ labeling bias, and pairwise correlation among samples via the arrayQualityMetrics package in Bioconductor (Kauffmann et al., 2009). CEL files were normalized via RMA (Irizarry et al., 2003), which includes global background adjustment and quantile normalization. Probes mapping to multiple genes were discarded, and the remaining probes were collapsed to gene level by selecting the probe for each gene with the highest mean expression across all subjects. Statistical tests were performed in MATLAB and R.

### Example 2

### Adjuvanting a Subunit SARS-CoV-2 Nanoparticle Vaccine to Induce Protective Immunity in Non-Human Primates

The development of a portfolio of SARS-CoV-2 vaccines to vaccinate the global population remains an urgent public health imperative. Here, we demonstrate the capacity of a subunit vaccine under clinical development, comprising the SARS-CoV-2 Spike protein receptor binding domain displayed on a two-component protein nanoparticle (RBD-NP), to stimulate robust and durable neutralizing antibody (nAb) responses and protection against SARS-CoV-2 in non-human primates. We evaluated five different adjuvants combined with RBD-NP including Essai O/W 1849101, a squalene-in-water emulsion; AS03, an alpha-tocopherol-containing squalene-based oil-in-water emulsion used in pandemic influenza vaccines; AS37, a TLR-7 agonist adsorbed to Alum; CpG 1018-Alum (CpG-Alum), a TLR-9 agonist formulated in Alum; or Alum, the most widely used adjuvant. All five adjuvants induced substantial nAb and CD4 T cell responses after two consecutive immunizations. Durable nAb responses were evaluated for RBD-NP/AS03 immunization and the live-virus nAb response was durably maintained up to 154 days post-vaccination. AS03, CpG-Alum, AS37 and Alum groups conferred significant protection against SARS-CoV-2 infection in the pharynges, nares and in the bronchoalveolar lavage. The nAb titers were highly correlated with protection against infection. RBD-NP immunization with AS03, AS37 and CpG-Alum groups cross-neutralized B.1.1.7 UK variant efficiently but showed a reduced response against the B.1.351 (SA variant). Of note, the AS03 group showed only a 4.5-fold reduction in cross-neutralization of the B.1.351 strain, while the AS37 group showed a 16-fold reduction, suggesting that different adjuvants vary in their capacity to induce nAbs that provide a greater breadth of neutralization. Furthermore, RBD-NP when used in conjunction with AS03 was as potent as the prefusion stabilized Spike immunogen, HexaPro. Taken together, these data highlight the efficacy of the RBD-NP formulated with clinically relevant adjuvants in promoting robust immunity against SARS-CoV-2 in non-human primates.

Subunit vaccines are amongst the safest and most widely used vaccines ever developed. They have been highly effective against a multitude of infectious diseases such as Hepatitis-B, Diphtheria, Pertussis, Tetanus and Shingles in diverse age groups, from the very young to the very old. An essential component of subunit vaccines is the adjuvant, an immune-stimulatory agent which enhances the magnitude, quality and durability of the immune responses induced by vaccination even with lower doses of antigen. Therefore, the development of a safe and effective subunit vaccine against SARS-CoV-2 would represent an important step in controlling the COVID-19 pandemic. The most widely used adjuvant, Aluminium salts (Alum), has been used in billions of doses of vaccines over the last century. During the past decade, several novel adjuvants have been developed including the α-tocopherol containing squalene-based oil-in-water adjuvant AS03, and the toll-like receptor (TLR)-9 ligand CpG 1018, which are included in licensed vaccines against pandemic influenza and Hepatitis-B, respectively. Alum has been known to skew immune responses to Th2-type, more so in mice, which was a potential concern for many vaccine makers to include Alum in SARS-CoV-2 vaccines. Oil-in-water emulsions such as AS03, on the other hand, are known to induce a more balanced Th1/Th2-type responses and elicit high magnitude of antibody responses making them a more attractive target for adjuvanting SARS-CoV-2 vaccines. The Toll-like receptor (TLR) agonists such as AS37 (TLR-7 agonist) and CpG 1018 (TLR-9 agonist) are known to induce efficient Th1-type immune response and potent antibody responses; however, their effects when formulated with Alum are not well established. In particular, under the commitment of GSK and Dynavax, AS03 and CpG 1018 are currently being developed as adjuvants for use in candidate subunit SARS-CoV-2 vaccines; however, their capacity to stimulate protective immunity against SARS-CoV-2 remains unknown.

We recently described SARS-CoV-2 RBD-16GS-I53-50 (RBD-NP), a subunit vaccine in which 60 copies of the SARS-CoV-2 RBD are displayed in a highly immunogenic array using a computationally designed self-assembling protein nanoparticle (hereafter designated RBD-NP). Pre-clinical evaluation in mice showed that the vaccine elicits 10-fold higher nAb titers than the two-proline (2P) prefusion-stabilized spike (which is used by most vaccines being developed) at a 5-fold lower dose and protects mice against mouse-adapted SARS-CoV-2 challenge. In the current study, we evaluated the capacity of AS03, CpG 1018 formulated in Alum, as well as the squalene-in-water emulsion (O/W), the TLR-7 agonist adsorbed to Alum (AS37) and Alum to promote protective immunity against SARS-CoV-2 in non-human primates (NHPs).

### Results

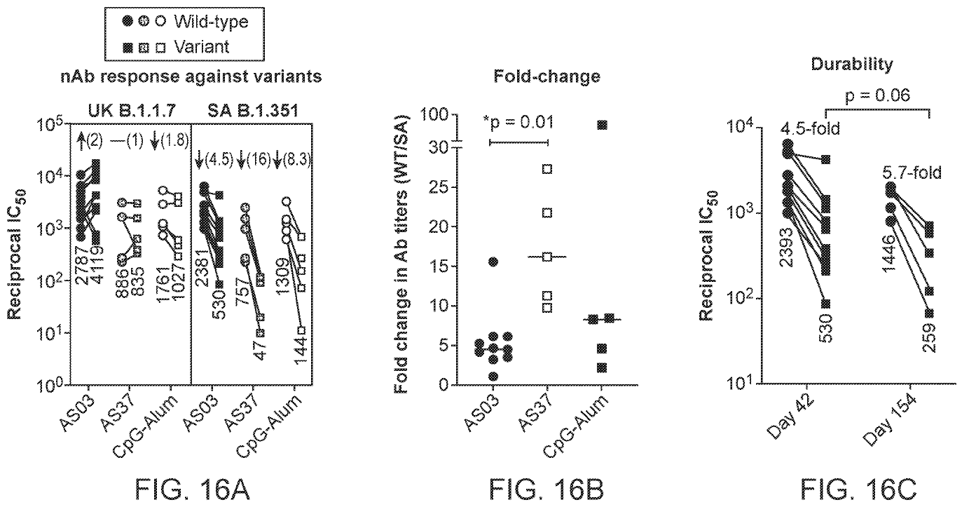

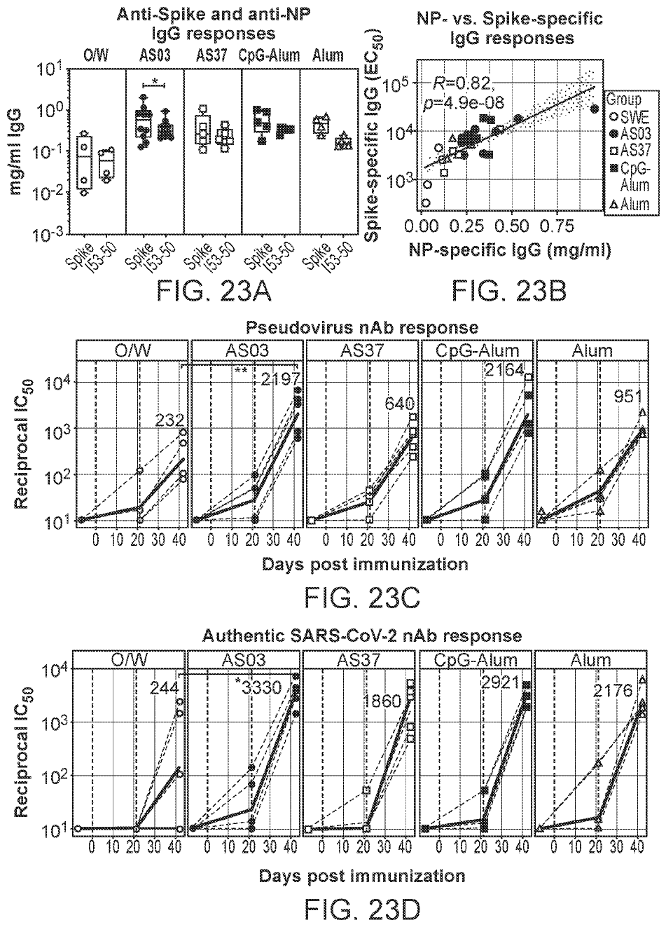

Robust antibody responses to RBD-NP formulated with different adjuvants. To assess the immunogenicity and protective efficacy of RBD-NP vaccination with different adjuvants, we immunized 29 male Rhesus macaques (RMs) with 25 μg RBD antigen (71 μg of total RBD-NP immunogen; FIG. 23) formulated with one of the following five adjuvants: O/W, AS03, AS37, CpG 1018-Alum (CpG-Alum) or Alum (FIG. 16a). Four additional animals were administered with saline as a control. All the immunizations were administered via intramuscular route on days 0 and 21 in forelimbs. Four weeks after the booster immunization, we challenged the animals with SARS-CoV-2 via intratracheal/intranasal (IT/IN) routes. Five of the ten animals immunized with AS03-adjuvanted RBD-NP were not challenged to allow evaluation of the durability of the vaccine-elicited immune responses and will be challenged at a distal time point.

Evaluation of binding antibody responses to vaccination showed that S-specific IgG was detected 21 days after primary immunization in all vaccination groups and increased in magnitude after boosting (FIG. 16b). AS03 induced the highest magnitude of binding IgG (GMT EC₅₀ 1:8,551) on day 42, and O/W induced the lowest (GMT EC₅₀ 1:1,308) response. Binding antibodies in the AS37, CpG-Alum, and Alum groups were comparable to AS03 in magnitude. In addition to S-specific IgG, we also measured antibody response to the 153-50 protein nanoparticle (NP) scaffold. Anti-NP antibody titers were elicited in all the groups albeit at a lower magnitude (1.7-fold lower on average) in comparison to the anti-Spike antibody titers among the different adjuvant groups at day 42 (FIG. 23a). The anti-NP antibody response correlated strongly with S-specific binding antibody responses (FIG. 23b).

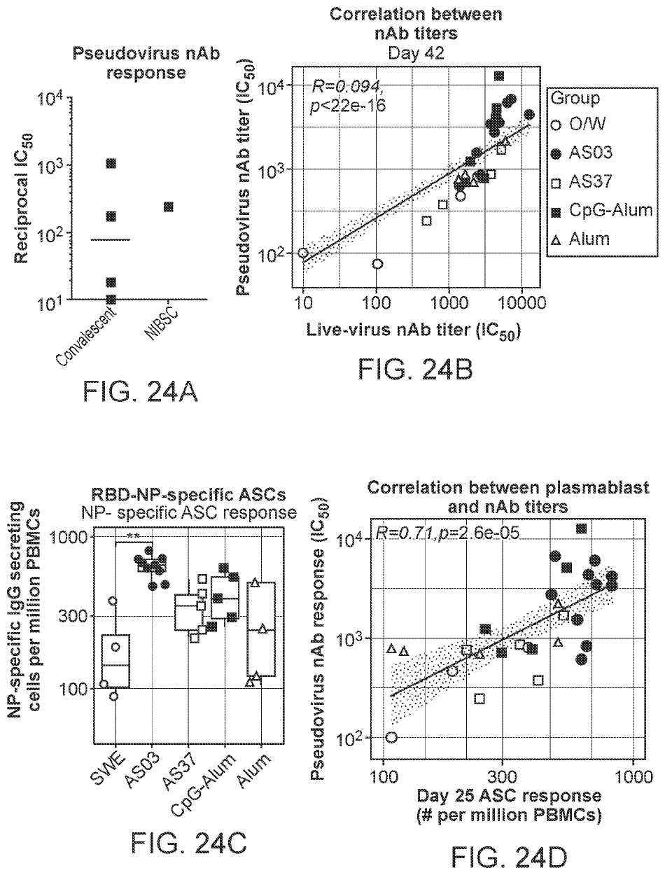

RBD-NP immunization induced detectable nAb responses against a SARS-CoV-2 S pseudotyped virus in most animals except in O/W group after primary immunization, which significantly increased in all groups after the booster immunization (FIG. 16c and FIG. 23c). In particular, the RBD-NP/AS03 immunization induced a geometric mean titer (GMT) of 1:63 on day 21 (3 weeks after primary immunization) that increased to 1:2,704 (43-fold) on day 42. The other groups, O/W, AS37, CpG-Alum, and Alum induced a GMT of 1:232, 1:640, 1:2,164, and 1:951 on day 42, respectively. These responses were remarkably higher than the nAb titers of 4 convalescent human samples (GMT 1:76) and the NIBSC control reagent (NIBSC code 20/130, nAb titer 1:241) (FIG. 24a) assayed simultaneously. Next, we measured the nAb responses against the authentic SARS-CoV-2 virus using a recently established Focus Reduction Neutralization Titer (FRNT) assay, which was used to analyze the recent clinical trials of the Moderna mRNA vaccine. Consistent with the pseudovirus neutralization assays, all adjuvants induced robust live-virus nAb titers after the secondary immunization (FIG. 16d and FIG. 23d). The RBD-NP/AS03 group showed the highest nAb titers (GMT 1:4,145) followed by the rest of the adjuvants. Furthermore, there was a strong correlation between pseudovirus and live-virus nAb titers, as seen in other studies (FIG. 24b). Lastly, we measured the RBD-NP-specific plasmablast response using ELISPOT four days after secondary immunization (FIG. 24c). The magnitude of antigen-specific IgG-secreting cells in blood correlated with the observed antibody responses (FIG. 24d).

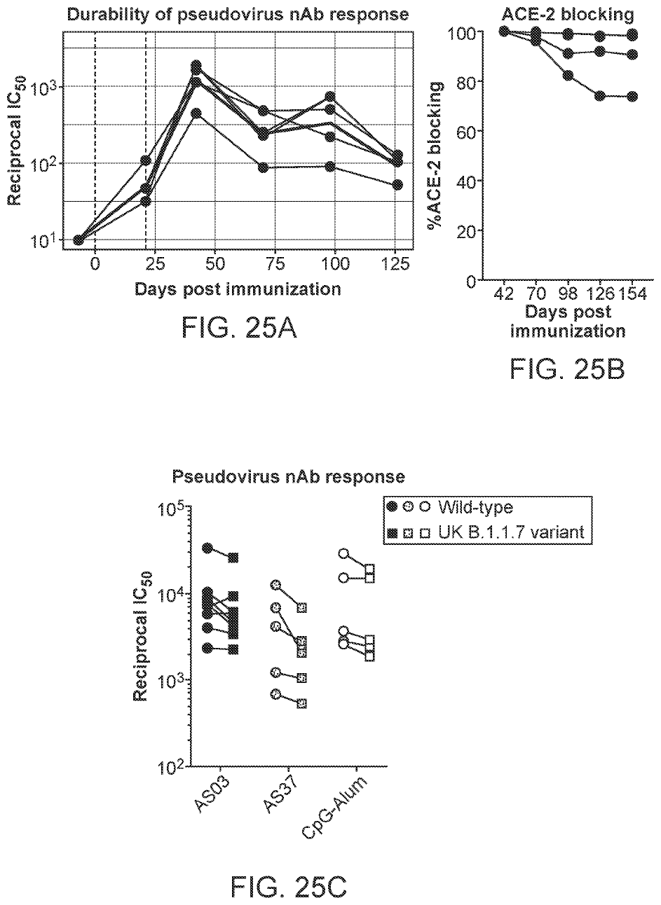

RBD-NP/AS03 immunization induces durable live-virus nAb responses. Inducing potent and durable immunity is critical to the success of a vaccine and determines the frequency with which booster immunizations need to be administered. To determine durability of nAb responses, we followed five animals immunized with RBD-NP/AS03 without challenge for 5 months. The pseudovirus nAb titers measured until day 126 declined moderately but did not differ significantly between days 42 and 126 (FIG. 25a). Strikingly, nAb response measured against the authentic SARS-CoV-2 virus using FRNT assay was durably maintained up to day 154 (FIG. 16e). Of note, the FRNT assay was performed in the same laboratory that measured durability in the Moderna vaccine study. The GMT titers decreased by 5-fold between day 42 (GMT 5.638 in the 5 animals that were followed) and day 154 (GMT 1,108), although this was not statistically significant (FIG. 16e). Furthermore, we observed little to no reduction in the efficiency of blocking of ACE-2 binding to RBD by sera collected at these time points (FIG. 25b). These results demonstrate that the RBD-NP/AS03 immunization induces potent and durable nAb responses.

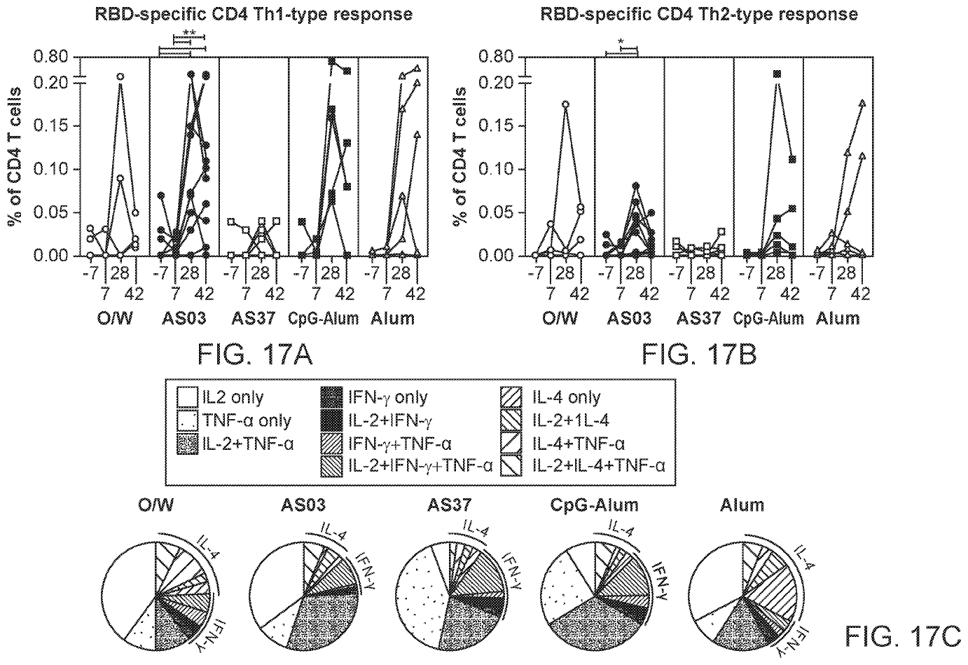

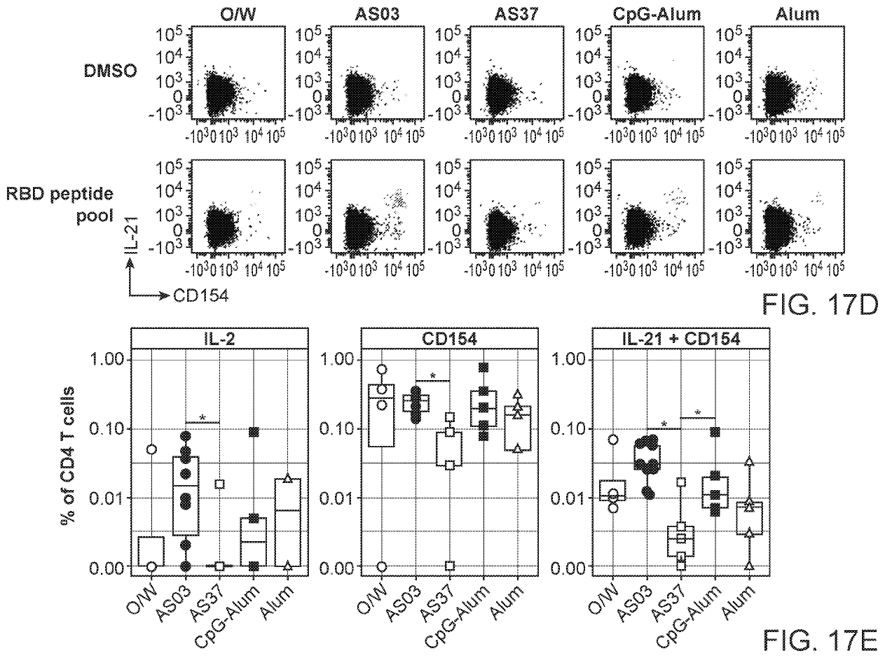

Adjuvanted RBD-NP immunization elicits nAb response against emerging variants. Variants of SARS-CoV-2 have been emerging recently, causing concerns that vaccine-induced immunity may suffer from a lack of ability to neutralize the variants. Two variants, B.1.1.7 and B.1.351, were first identified in the United Kingdom and South Africa, respectively, and have since been found to be circulating globally. We evaluated if sera from animals immunized with RBD-NP+AS03, AS37, or CpG-Alum, neutralizes the B.1.1.7 and B.1.351 variants. Using live-virus neutralization as well as pseudovirus neutralization assays, we determined that all the three groups induced nAb titers against the variants. While the nAb titers against the B.1.1.7 variant was comparable to that of the wild-type (WT) SARS-CoV-2 (FIG. 16a, left panel and FIG. 25c), the nAb titers against the B.1.351 South African variant was significantly reduced in comparison to that of WT (FIG. 17a, right panel, Table 1) as seen in vaccinated humans. Notably, the reduction was higher in the AS37 group (median 16-fold) compared to the AS03 (4.5-fold) and CpG-Alum (8.3-fold) groups. These data suggest that the adjuvants not only enhance immunogenicity, but different adjuvants may vary in their potential to elicit nAbs that provide a greater breadth of neutralization. Furthermore, the nAb response against the B.1.351 variant was as durable as that of the WT responses (FIG. 17c).

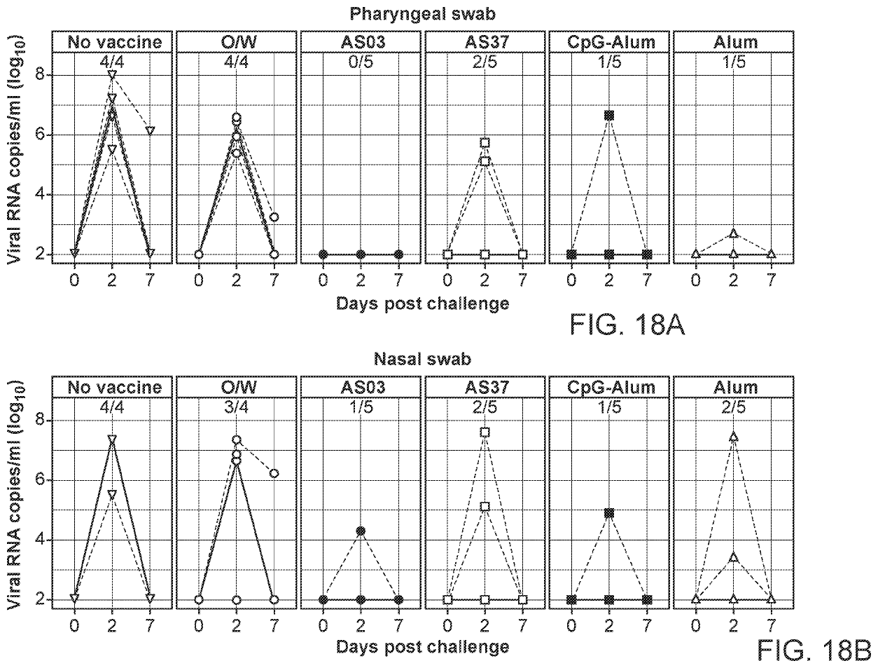

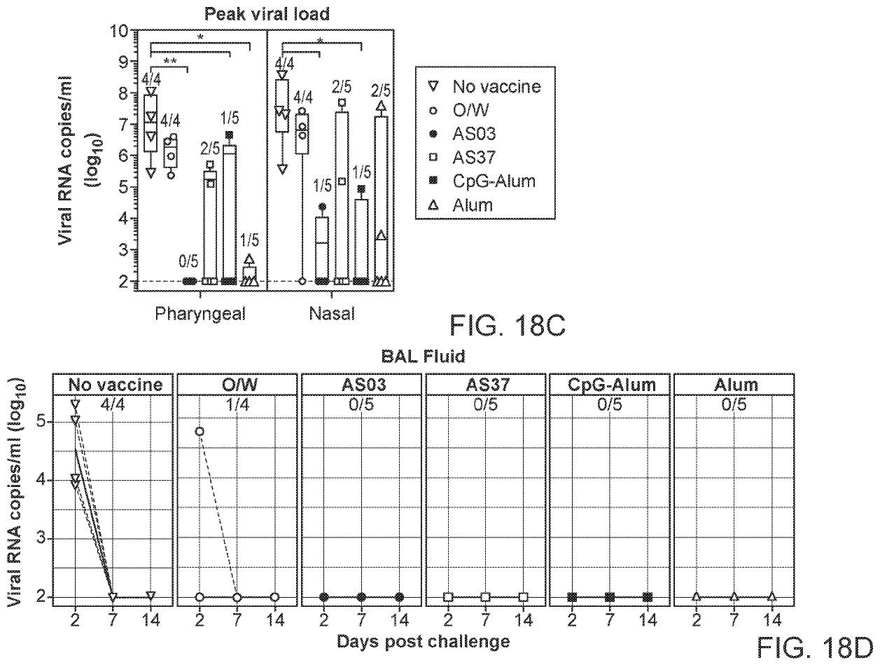

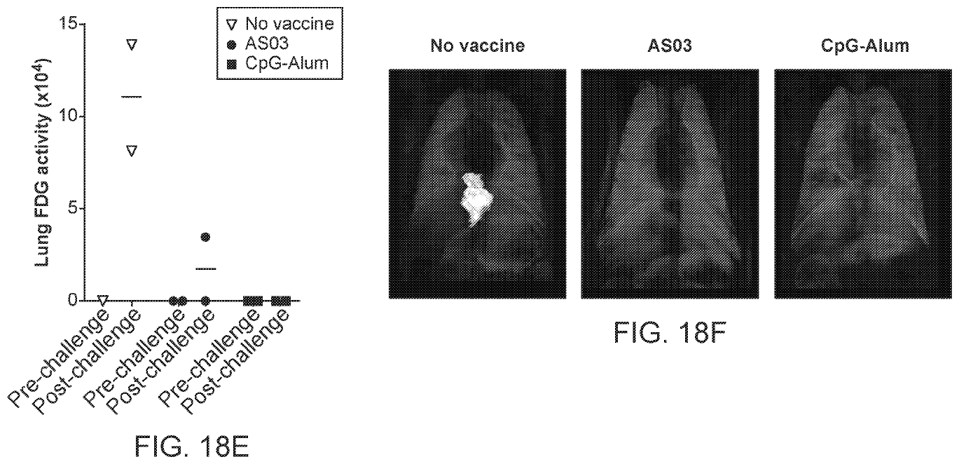

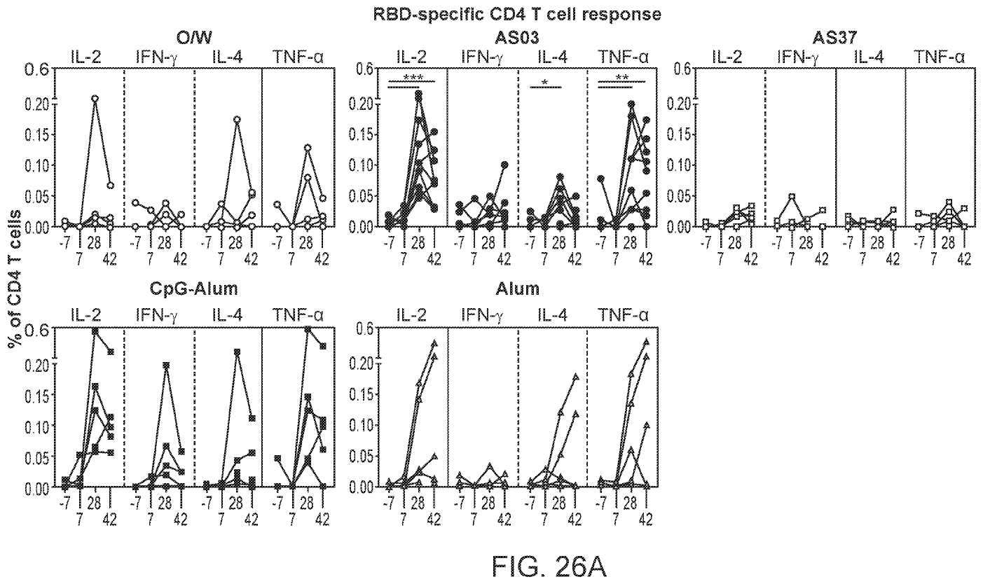

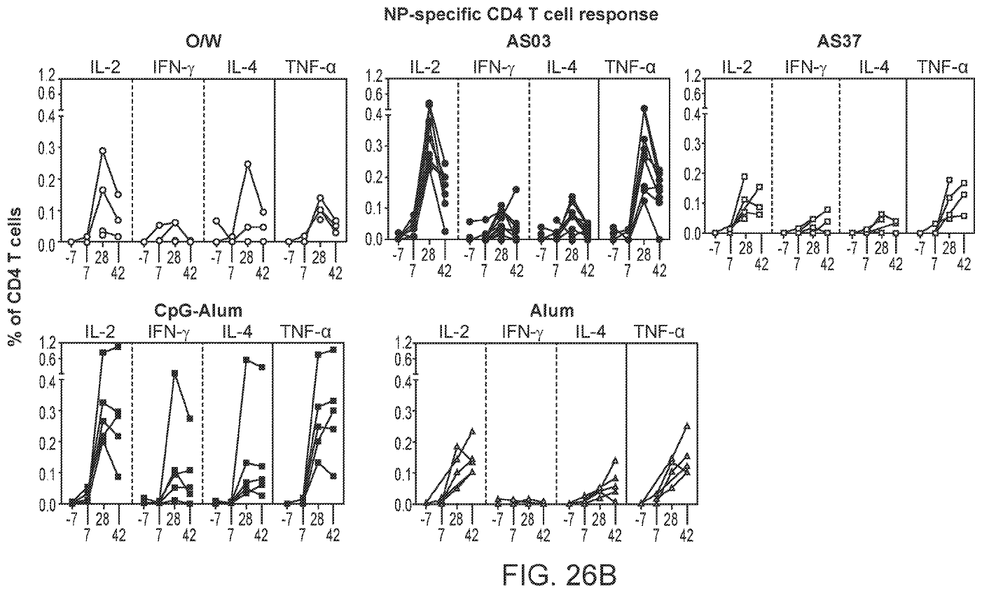

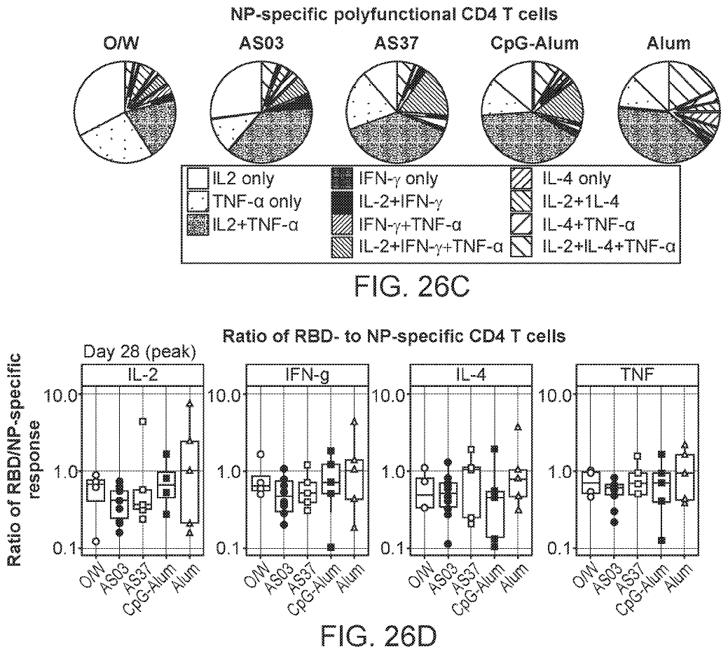

Adjuvanted RBD-NP immunization induces robust CD4 T cell responses. We assessed antigen-specific T cell responses by intracellular cytokine staining (ICS) assay using a 21-parameter flow cytometry panel (Supplementary Table. 2). We first measured RBD-specific T cells after ex vivo stimulation with a peptide pool (15-mer peptides with 11-mer overlaps) spanning the SARS-CoV-2 RBD. RBD-NP immunization induced an antigen-specific CD4 T cell response but limited CD8 T cell response. RBD-specific CD4 responses were highest in the AS03 and CpG-Alum groups (FIG. 18a, b), and were significantly enhanced after secondary immunization. These responses were dominated by IL-2 or TNF-α-secreting CD4 T cells (FIG. 26a), which remained detectable at day 42 (3 weeks post-secondary immunization). The median frequencies of IL-2⁺ and TNF-α⁺ CD4 T cell responses in the AS03 group were 0.1% and 0.08%, respectively, on day 28 and reduced to ˜0.07% on day 42. There was also a low but detectable IL-4 response in both the AS03 and CpG-Alum groups that peaked on day 28 but declined nearly to baseline levels by day 42 (FIG. 18b). Next to AS03 and CpG-Alum groups, Alum also induced a potent CD4 T cell response. Whereas 75% and 50% of animals in the Alum and O/W groups showed induction of RBD-specific CD4 T cells, respectively, the TLR-7 agonist AS37 induced a weak T cell response despite inducing potent antibody response in all the animals.

We assessed the polyfunctional profile of antigen-specific CD4 T cells expressing IL-2, IFN-γ IL-4, and TNF-α (FIG. 18c). Although IL-2⁺, TNF-α⁺, and IL-2⁺INF-α⁺ double-positive cells formed the majority (˜70%) in all adjuvant groups, differences between the groups were apparent. In particular, AS03 elicited similar proportions of polyfunctional Th1-type and Th2-type CD4 T cells, a balanced Th1/Th2 profile, CpG-Alum showed a slightly higher Th1-type response, and Alum a higher Th2-type response. We further extended our analyses to measure IL-21 and CD154, markers of circulating T_{FH}-like cells for their critical role in germinal center formation and generation of durable B cell responses. We observed detectable IL-21 responses in the AS03 and CpG-Alum groups (FIG. 18d). All cells secreting IL-21 were CD154⁺. The IL-21⁺CD154⁺ double-positive cells were significantly higher in the AS03 and CpG-Alum groups in comparison with the AS37 group (FIG. 18e).

We also stimulated peripheral blood mononuclear cells (PBMCs) with a peptide pool spanning the 153-50A and 153-50B nanoparticle component sequences to determine if RBD-NP immunization induces T cells targeting the nanoparticle scaffold. We observed a significant proportion of CD4 T cells targeting the 153-50 subunits with a response pattern, including polyfunctional profiles, similar to that of the RBD-specific T cells (FIG. 26b,c). The frequencies of NP-specific CD4 T cells were ˜3-fold higher than that of RBD-specific CD4 T cells (FIG. 26d), an observation that is consistent with the RBD making up approximately one third of the total peptidic mass of the immunogen. In summary, the RBD-NP immunization with adjuvants induced vaccine-specific CD4 T cells of varying magnitude. While IL-2 and TNF-α were the major cytokines induced by antigen-specific CD4 T cells, we also observed IL-21 and CD154 responses.

RBD-NP immunization with different adjuvants protects NHPs from SARS-CoV-2 challenge. The primary endpoint of the study was protection against infection with SARS-CoV-2 virus, measured as a reduction in viral load in upper and lower respiratory tracts. To this end, we challenged the animals four weeks post-secondary immunization with 3.2×10⁶ PFU units via intratracheal and intranasal (IT/IN) routes. Viral replication was measured by subgenomic PCR quantitating the E gene RNA product on the day of the challenge, as well as 2-, 7- and 14-days post-challenge in nares, pharynges and BAL fluid.

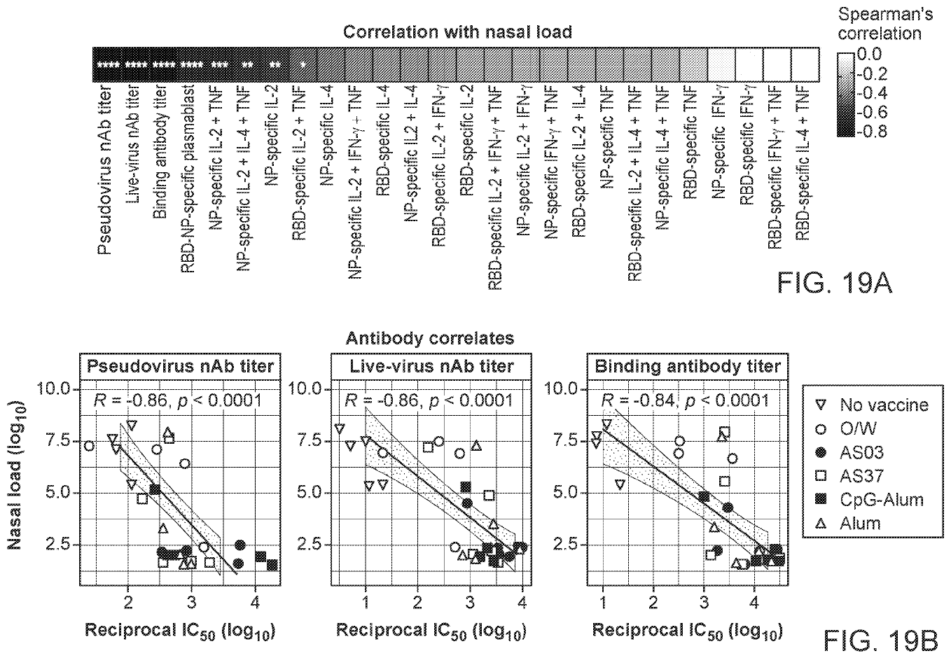

Two days after challenge, 4 out of 4 control animals had detectable subgenomic viral RNA (E gene, range 3.1×10⁵-3.5×10⁸ viral copies) in the pharyngeal and the nasal compartments. By day 7, the viral RNA quantities reduced to baseline, consistent with previous studies. All adjuvanted groups, except O/W, afforded protection from infection (FIG. 19a, b). In particular, none of the five animals challenged in the AS03 group had detectable viral RNA in pharyngeal swabs at any time and only one animal had detectable viral RNA in nasal swabs, at a level ˜1,000-fold lower than the median in control animals (2.2×10⁴ vs. 2.5×10⁷ viral copies). In contrast, viral RNA was detectable in pharyngeal swabs from all four animals in the O/W group, albeit at lower levels than the control group, and three out of four animals had detectable viral RNA in nasal swabs. Only one out of five animals in the CpG-Alum group had detectable viral RNA in pharyngeal or nasal swabs. The AS37 group and, remarkably, the Alum group also showed undetectable viral RNA in 3 of the 5 animals in both compartments (FIG. 19c).

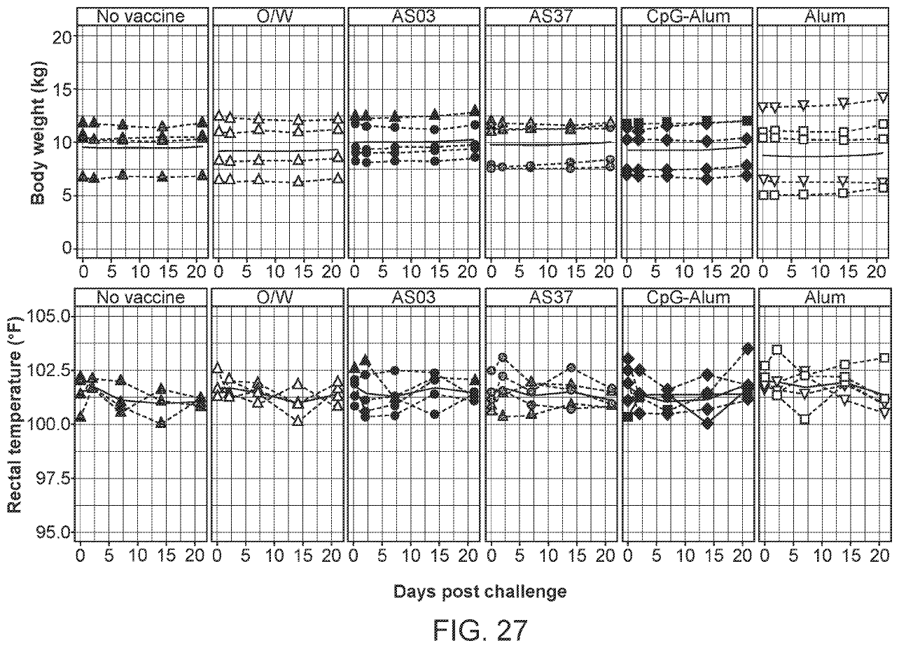

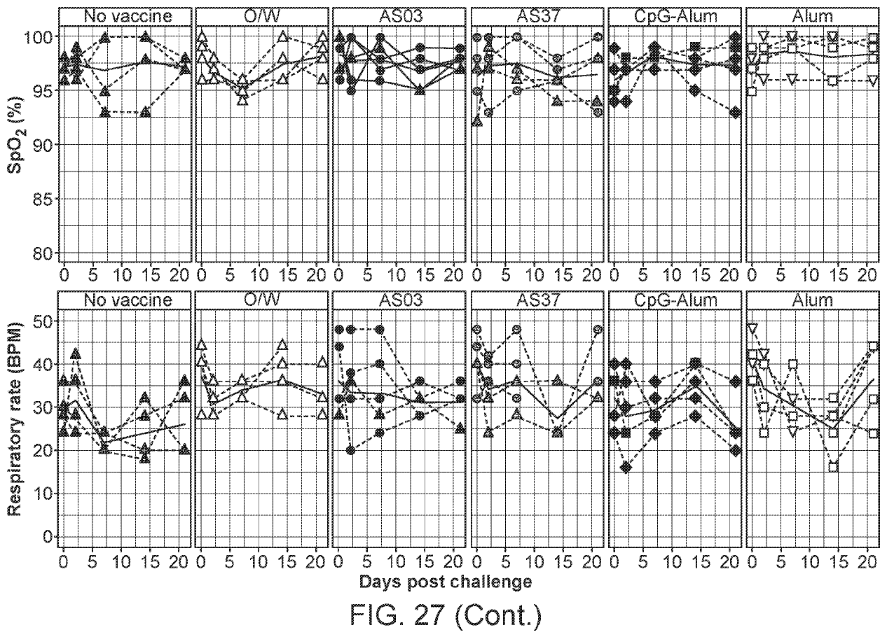

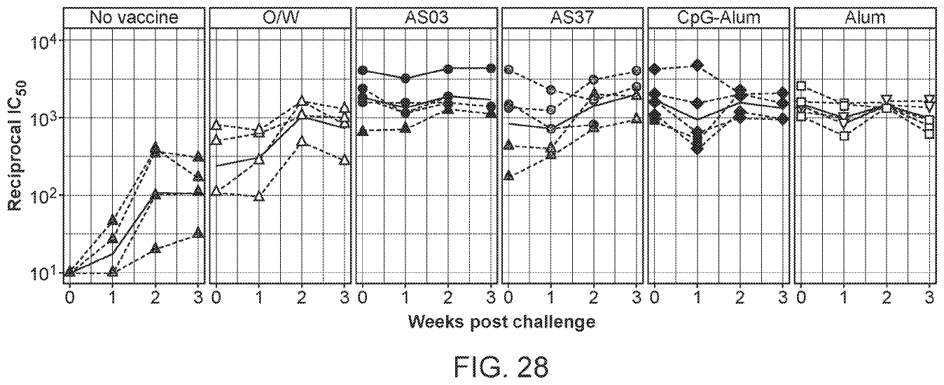

We measured the subgenomic viral RNA in bronchoalveolar lavage (BAL) fluid to assess protection in the lung. We used a more sensitive PCR assay measuring the N gene product as we found only 2 control animals showing a positive viral load in the BAL using E subgenomic RNA. Two days after the challenge, all 4 of the four control animals showed a viral load in the range of 10⁴-10⁶ viral copies. In contrast, none of the animals in the vaccinated groups except one animal in the O/W group showed any detectable virus (FIG. 19d), suggesting effective protection in the LRT of all vaccinated groups, including the O/W group. There were no signs of clinical disease in any animals, whether or not vaccinated disease (FIG. 27); however, the control animals but not vaccinated animals responded with an increase in nAb titers (FIG. 28), consistent with the idea that SARS-CoV-2 infection of Rhesus macaques results in a mild disease. Overall, the RBD-NP vaccination with adjuvants offered varying degree of protection against SARS-CoV-2 challenge in upper and lower respiratory tracts.

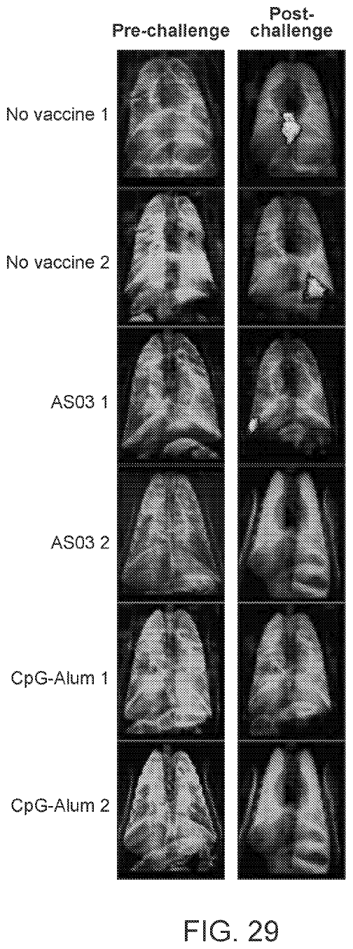

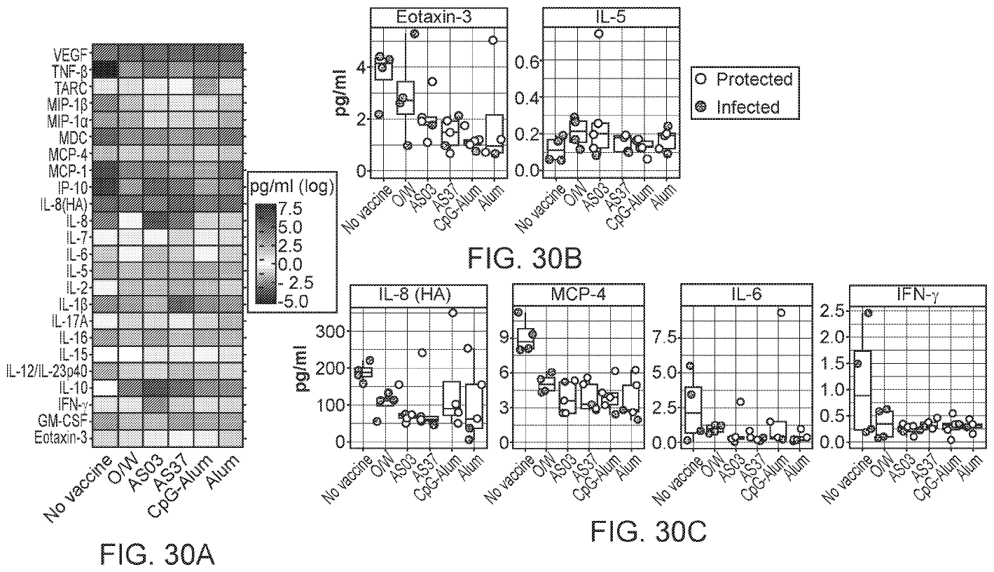

Vaccine-associated enhanced respiratory disease (VAERD) has previously been described for respiratory infections with respiratory syncytial virus and SARS-CoV. We evaluated inflammation in the lung tissues of a subset of animals using PET-CT on the day of the challenge and 4-5 days post-challenge. Of the six animals evaluated (2 from no vaccine, 2 from AS03, and 2 from CpG-Alum groups selected randomly), we found inflammation in both control animals on day four compared to baseline, as measured by enhanced 2-Deoxy-2-[18F]fluoroglucose (FDG) uptake. In contrast, only one of the four vaccinated animals showed FDG uptake, to a much lesser extent than the control animals (FIG. 19e and FIG. 29). Additionally, we evaluated inflammation in the lung by performing a comprehensive analysis of cytokine responses measuring 24 cytokines including Th2-polarizing and eosinophilic cytokines such as IL-5 and Eotaxin in all the animals one week post challenge. The data demonstrate that there was no enhanced inflammation in the lungs of any vaccinated animal (FIGS. 30a and b). There was also an increased abundance of cytokines such as IL-6, IL-8, IFN-γ and MCP-4 known to be induced by SARS-CoV-2 infection in humans in the lungs of control but not vaccinated animals (FIG. 30c). These data are consistent with an absence of VAERD in these animals.

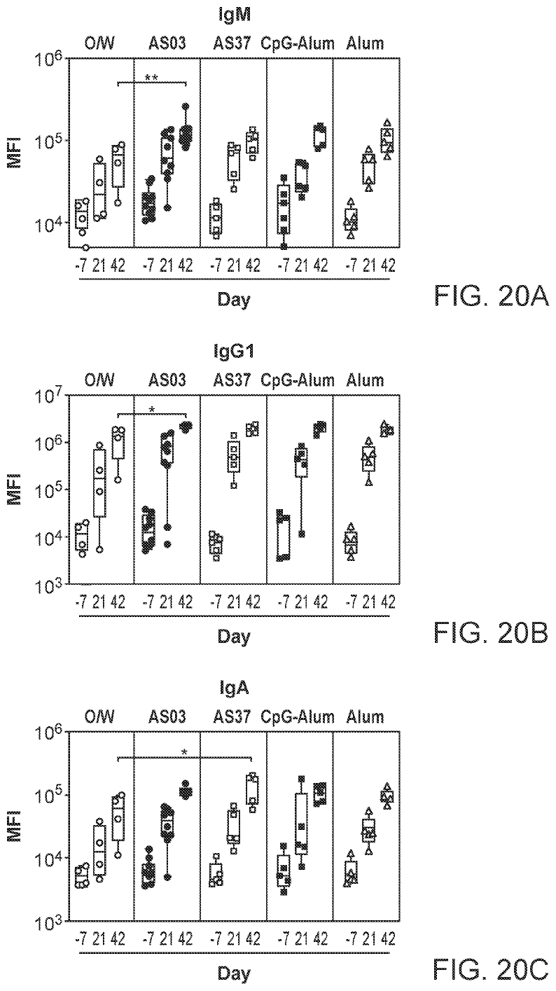

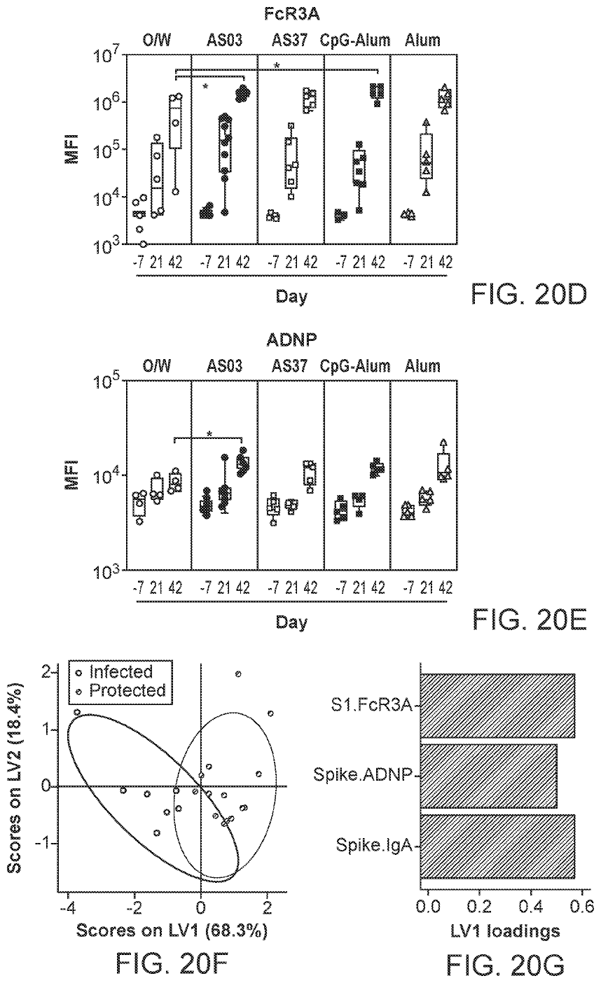

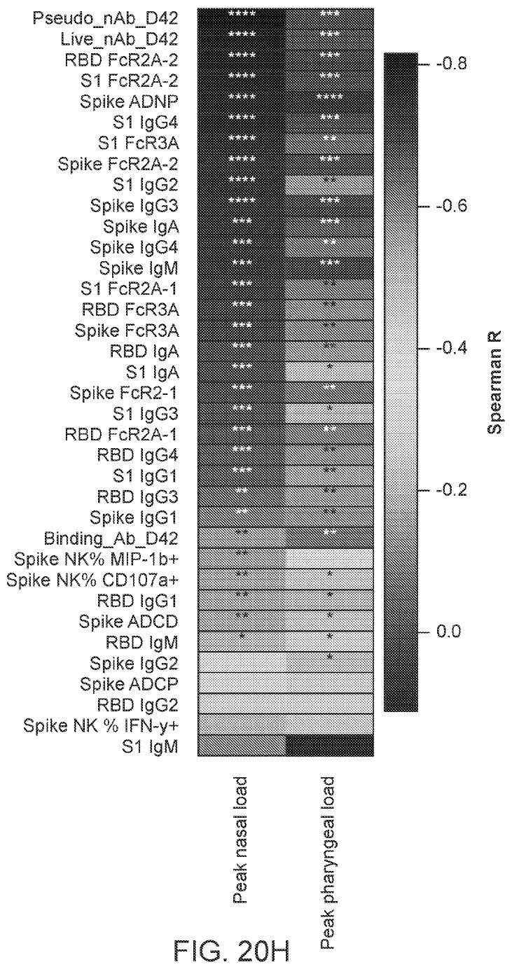

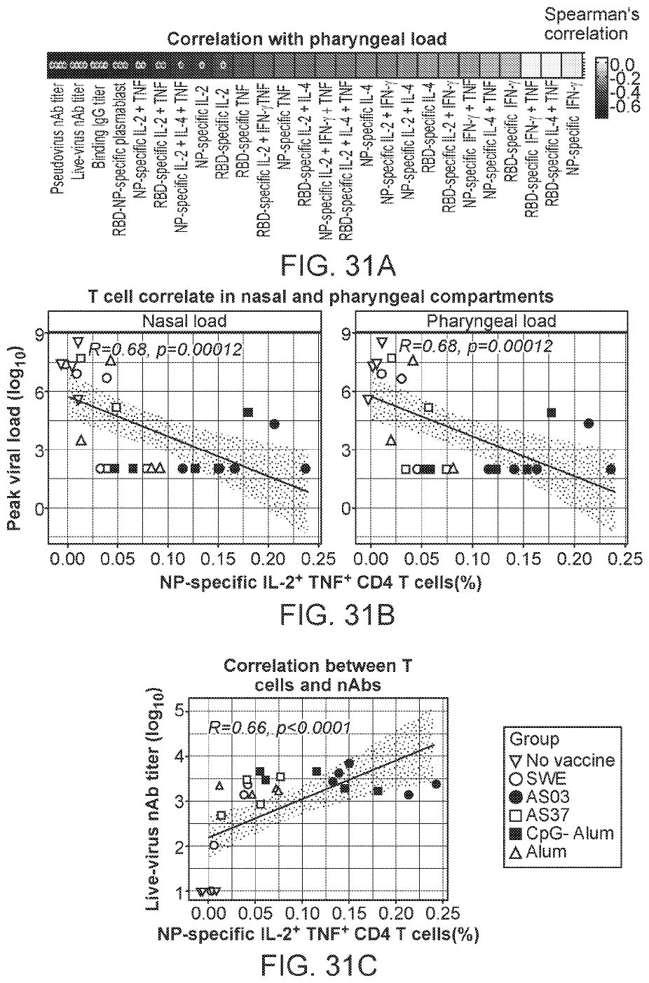

Immune correlates of protection. Next, we set out to identify immune correlates of protection. Since we had five different adjuvant groups showing different protection levels within each group, we analyzed the correlations by combining animals from all the groups. We correlated humoral and cellular immune responses measured at peak time points (day 42 for antibody responses and day 28 for T cell responses) with the viral load (nasal or pharyngeal) to determine the putative correlates of protection in an unbiased approach. Neutralizing, both live and pseudovirus, titers emerged as the top statistically significant correlates of protection (FIG. 20a, b, and FIG. 31a) in both nasal and pharyngeal compartments. Interestingly, NP-specific IL-2⁺TNF⁺CD4 T cell response also emerged as a statistically significant correlate of protection in both compartments (FIG. 20a and FIG. 31b), the frequencies of which positively correlated with nAb titers (FIG. 31c). This is consistent with the possibility that NP-specific CD4 T cells could offer T cell help to RBD-specific B cells.

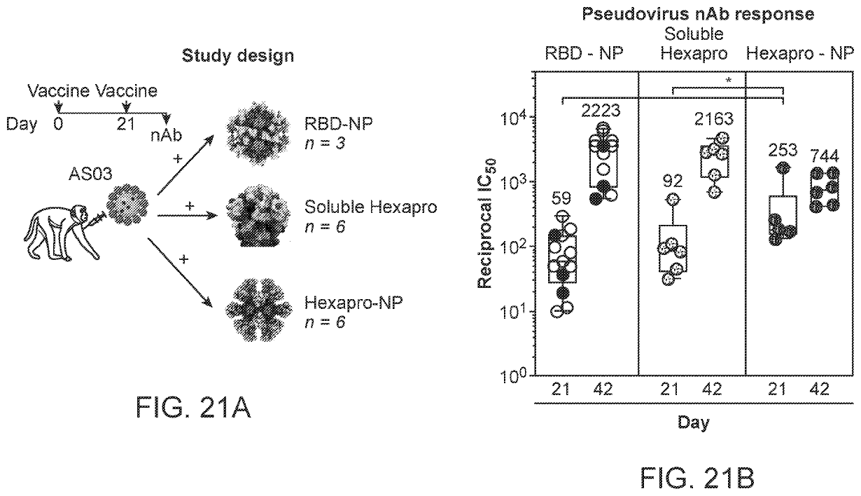

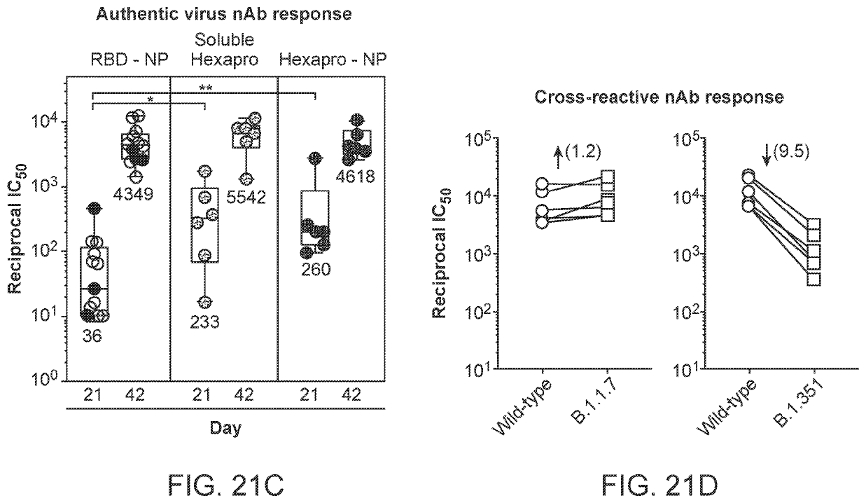

Systems serology profiling reveals functional antibody responses to RBD-NP vaccination. In addition to characterizing nAb and T cell responses to vaccination, we sought to understand the humoral functional profile elicited by each adjuvant. Vaccines rapidly induced a humoral immune response against SARS-CoV-2 spike with a profound increase in different anti-spike antibody isotypes (FIG. 21a-c) and FcR-binding (FIG. 21d) and ADNP (FIG. 21e) at day 21 and day 42. The IgM response followed the same pattern as IgG and IgA isotypes, a detectable response after primary immunization which was significantly boosted after the second dose (FIG. 21a) in all groups. AS03 has the highest response followed by the rest of the adjuvants. To understand how differences in the humoral response may lead to viral breakthrough, we performed a partial least square discriminant analysis (PLSDA) on the antibody features measured at day 42, using least absolute shrinkage and selection operator (LASSO) to select features to prevent overfitting (FIG. 21f). The PLSDA analysis showed separation between animals that had viral breakthrough in the nasal and pharyngeal and those that showed no viral breakthrough (FIG. 21f), marked by an enrichment in IgA, FcR3A and antibody-dependent neutrophil phagocytosis (ADNP) against spike in the protected animals (FIG. 21g).

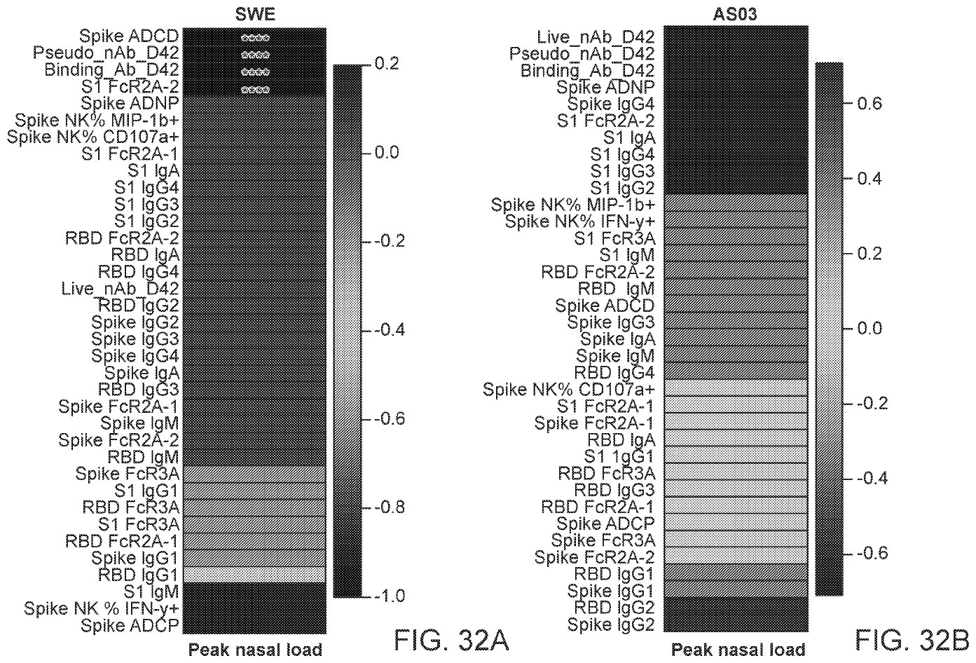

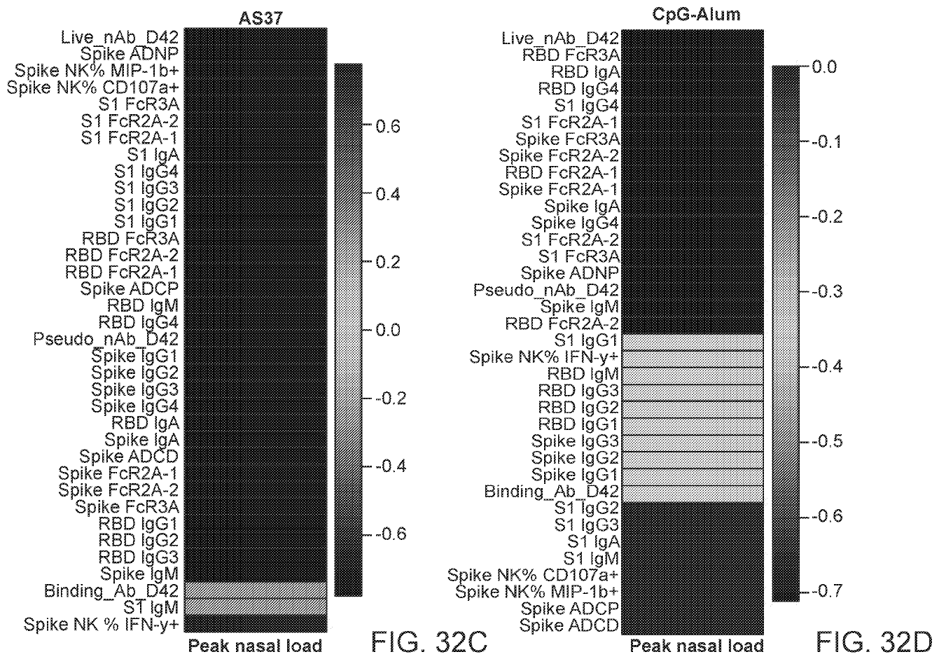

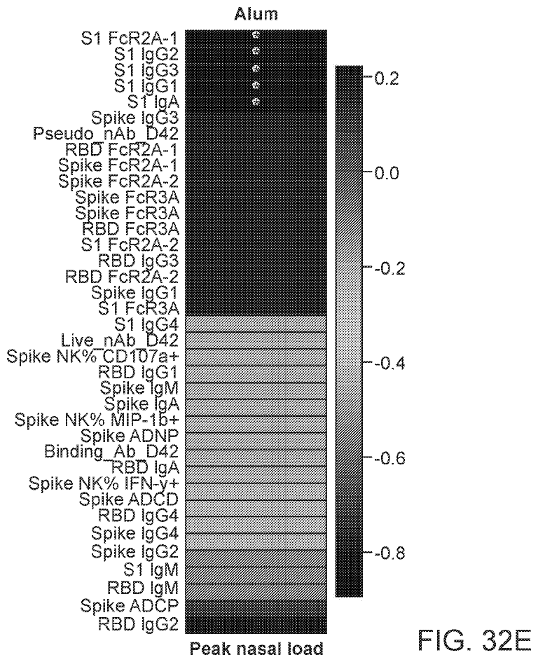

We determined the correlation of each measured antibody feature and the peak nasal and pharyngeal viral load to further dissect the antibody features that provide protection against viral break-through. Whereas neutralizing Ab response still represents the strongest correlate of protection, we observed additional functional features including FcR binding (RBD FcR2A-2 and 51 FcR2A-2), and antibody-dependent neutrophil phagocytosis (ADNP) that were negatively correlated with nasal or pharyngeal viral loads (FIG. 21h). These data demonstrated an additive role for functional antibody responses in protection. Furthermore, each adjuvant group mounted a distinct profile of antibody response that correlated with protection against the virus (FIG. 32). These differences between groups highlight that different adjuvants can elicit unique functional antibody responses to coordinate a protective antiviral response.

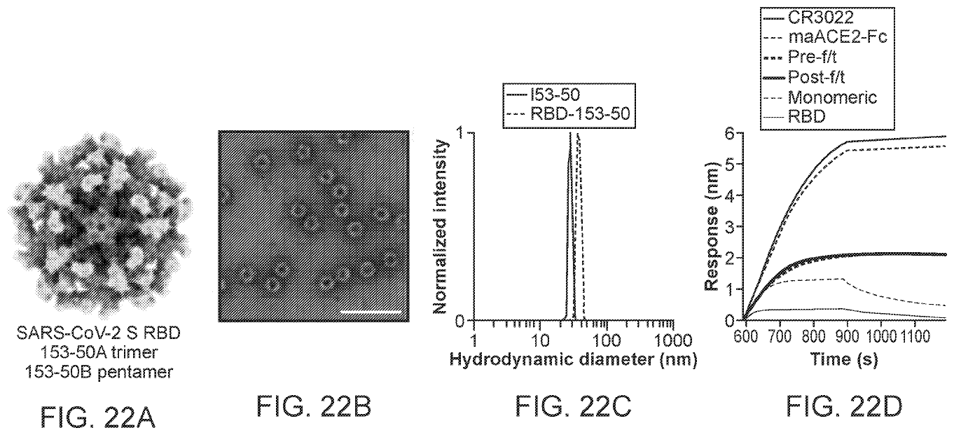

Comparison of different Spike-based immunogens with AS03. The data described thus far demonstrate that RBD-NP immunogen when adjuvanted with AS03, AS37, CpG-Alum and Alum induce robust protective immunity. As a next step, we compared the immunogenicity of the RBD-NP immunogen to that of HexaPro, a highly stable variant of the prefusion Spike trimer, in soluble or in a nanoparticle form. To this end, we designed a second study in which we immunized an additional 15 male RMs, distributed into three groups, with RBD-NP, soluble HexaPro or 20 Hexapro trimers displayed on the 153-50 nanoparticle (Hexapro-NP) (FIG. 22a). All three groups were adjuvanted with AS03, the adjuvant that provided the highest magnitude of nAb responses and protection in all animals in the upper and lower respiratory tracts in previous experiments. The RBD-NP/AS03 immunization induced nAb titers comparable to that of the previous study, with a detectable titer on day 21 that boosted robustly at day 42. In comparison to the RBD-NP, soluble Hexapro or Hexapro-NP immunization induced notably higher nAb titers against the matched pseudovirus or authentic virus after one immunization (FIG. 22b, c). The RBD-NP, however, boosted strongly such that the magnitude of the nAb titers was not statistically different between the three groups on day 42 (FIG. 22b, c). Furthermore, soluble Hexapro immunization with AS03 also elicited cross-reactive nAb response against the B.1.1.7 and B.1.351 variants (FIG. 22d), as was the case for RBD-NP (FIG. 17a). Taken together, these data indicate that the RBD-NP was as potent an immunogen as this highly stable version of the prefusion Spike trimer, consistent with previous observations that the vast majority of the neutralizing antibody response elicited by infection or immunization with trimeric Spike targets the RBD. Moreover, these data suggest AS03 can be considered as a suitable adjuvant for clinical use with various forms of the Spike protein.

The recent emergency use authorization of two messenger RNA (mRNA) vaccines against SARS-CoV-2 represents a major milestone in the fight against the COVID-19 pandemic. However, manufacturing several billion doses of vaccines to vaccinate the entire world's population will require a portfolio of different vaccine candidates. In particular, vaccinating special populations such as infants and the elderly could benefit from the use of subunit adjuvanted vaccine platforms with a demonstrable history of safety and efficacy in such populations. The primary objective of this study was to select adjuvants for clinical development of the novel RBD-NP subunit vaccine candidate. We evaluated five different adjuvants, including two, Alum and AS03, that have been used in several millions of doses of licensed vaccines, for their capacity to elicit enhanced responses with the SARS-CoV-2 RBD-NP immunogen. All adjuvants tested induced substantial nAb titers (FIG. 16c, d). Surprisingly, O/W induced relatively lower nAb titers compared to the other adjuvants. O/W is an oil-in-water emulsion similar to AS03 but does not contain α-tocopherol, a potent immunomodulator shown to be required to achieve high magnitude of antibody responses. While the absence of α-tocopherol could be an explanation for a lack of response after primary immunization and the relatively lower titers induced after boosting in the O/W group, parallel studies in mice demonstrated O/W was as immunogenic as AS03. Alum, on the other hand, induced nAb responses statistically comparable to that of AS03. While the peak immune responses were comparable, future studies should aim to characterize long-lasting immunity to vaccination with Alum. In general, all adjuvants induced detectable antigen-specific CD4 T cell responses, with AS03 and CpG-Alum inducing the highest frequencies. A notable finding was the induction of a high magnitude of CD4 T cell responses specific to the NP-scaffold. It is likely that these NP-scaffold-specific CD4 T cells could provide T cell help to RBD-specific B cells and promote B cell responses.

Concomitant with these immune responses, we observed varying levels of protection against IN/IT challenge with SARS-CoV-2 virus in the various adjuvant groups. Furthermore, we did not observe any inflammation in the lungs 4 days post-challenge precluding the possibility of VAERD. The varying responses observed between the different groups allowed us to analyze putative correlates of protection. Using an unbiased correlation approach, we determined the nAb response as the primary correlate of protection; however, NP-specific IL-2⁺/INF⁺ responses also showed a correlation. While this correlation is because of an indirect effect of correlation between T cells and nAb responses as showed in FIG. 31C, or that the T cell represent an independent correlate of protection by synergizing with nAb response needs to be ascertained in future studies. Finally, and importantly, the nAb response induced by RBD-NP/AS03 immunization was durable and led to efficient blocking of viral spike RBD binding to human ACE2 for up to 5 months post-immunization. SARS-CoV-2 spike RBD mediates viral infection by binding to host membrane receptors, with ACE2 being described as the primary receptor for viral cell entry. It remains to be investigated if vaccine responses provide similar durable blocking activities in the host in the context of host protease activation of spike protein and the presence of other host cell receptors that may have a role in SARS-CoV-2 entry such as the transmembrane glycoprotein CD147 (basigin), and the 78 kDa glucose-regulated protein (GRP78) receptor. Overall, the magnitude of nAb responses induced in the AS03 and CpG-Alum groups were comparable to that of Moderna and Pfizer mRNA vaccines in macaques. The Alum and AS37 groups were not too far behind but O/W elicited only a moderate response. In contrast to the Moderna (mRNA1273) where the authors observed no Th2-type responses, we observed a Th2 (IL-4) response in CD4 T cells. There was also a difference in the Th1/Th2 profiles induced by different adjuvants. While AS03 induced a balanced mix of Th1 and Th2 type response, CpG-Alum showed a higher Th1-type response and Alum skewed the response towards Th2 type. A modest IL-4 response was also seen in macaques immunized with the Pfizer vaccine, BNT162b2 which has been demonstrated to be safe in humans. Furthermore, we saw no evidence of VAERD in the challenged animals suggesting that the Th2 type response may not be a concern in humans. The adjuvanted RBD-NP immunization also induced cross-neutralization of emerging variants B.1.1.7. as well as the SA strain B.1.351, that appears to be more resistant to neutralization by convalescent serum indicating immune escape. While nAb responses in all three groups showed an overall decline, the AS37 group (16-fold) showed a steeper decline in comparison to the AS03 group (4.5-fold) (FIG. 17b and Table 1) indicating that different adjuvants may vary in their ability to promote breadth of neutralization by antibodies. While further experiments are warranted to confirm these findings and delineate the mechanisms underlying these results, our observation that different adjuvants can promote varying degrees of breadth of nAb responses may have a broader relevance for vaccinology in general.

In addition to evaluating clinically relevant adjuvants, we also compared the immunogenicity of RBD and prefusion-stabilized trimeric Spike immunogens. Our results demonstrate that the RBD-NP immunogen is as potent as immunogens based on the prefusion Spike trimer in inducing nAb titers. Whether differences in immunogenicity become apparent at lower doses of antigen warrants further investigation. Nonetheless, these data are encouraging as vaccine candidates in both antigenic formats (i.e., RBD vs. prefusion-stabilized trimeric Spike) were highly immunogenic with AS03, each with distinct manufacturing considerations, move forward to the clinic. Of particular interest to the field will be to evaluate whether the nAb responses elicited by RBD-NP or HexaPro-based immunogens induces breadth not only against the new SARS-CoV-2 variants, but also against other coronaviruses. We have not tested other adjuvants with Spike immunogens. It is formally possible that the immunogenicity may differ between immunogens when combined with different adjuvants.

Overall, the current study represents the most comprehensive comparative immunological assessment of a set of clinically relevant vaccine adjuvants and antigens in promoting robust and highly efficacious immune responses against a candidate subunit SARS-CoV-2 vaccine. These data reveal the promising performance of several adjuvants including AS03 and CpG 1018 (with Alum), which have been used in licensed vaccines, when used in conjunction with the SARS-CoV-2 RBD-NP immunogen. These results bode well for the clinical development of RBD-NP and other SARS-CoV-2 subunit vaccines with these adjuvants.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method for modulating the epigenome of innate immune cells, the method comprising:
administering an immunostimulatory composition comprising an adjuvant to an individual to stimulate broad and persistent innate immunity against pathogens unrelated to antigens present in the composition.

2. The method of claim 1, wherein the immunostimulatory composition comprises adjuvant but lacks additional antigens.

3. The method of claim 1, wherein the immunostimulatory composition consists of an adjuvant.

4. The method of claim 1, wherein the immunostimulatory composition comprises adjuvant and a non-pathogen antigen, or an antigen derived from a pathogen.

5. The method of claim 1, wherein the immunostimulatory composition comprises adjuvant and a antigenic material for seasonal or pandemic influenza virus.

6. The method of claim 1, wherein the adjuvant is a water-in-oil emulsion.

7. The method of claim 6, wherein the emulsion comprises squalene, optionally AS03 and/or MF59.

8. (canceled)

9. The method of claim 1, wherein the adjuvant is a TLR ligand, including a TLR7/8 or TLR3 or TLR4 ligand, or any combination thereof.

10. The method of claim 1, wherein the adjuvant is encapsulated in a nanoparticle or in other formulations.

11. The method of claim 1, wherein the adjuvant stimulates an epigenetic state in monocytes, mDCs or myeloid cells that imprints enhanced antiviral resistance and/or makes them refractory to producing pro-inflammatory cytokines such as TNF, IL-6, IL-1 beta.

12. (canceled)

13. The method in claim 12 for use to stimulate an epigenetic state in monocytes and mDCs to suppress excessive inflammation or sepsis in infection.

14. (canceled)

15. The method of claim 1, wherein administration is prophylactic for a viral infection optionally performed prior to a period of time in which the individual will be at increased risk of pathogen exposure.

16. (canceled)

17. The method of claim 1, wherein an individual selected for administration has reduced adaptive immune responses.

18. The method of claim 1, wherein administration is repeated at suitable intervals as the immune responsive state fades.

19. The method of claim 1, wherein the individual is monitored for epigenetic changes in myeloid cells, optionally classical monocytes and myeloid dendritic cells.

20. (canceled)

21. The method of claim 19, wherein a responsive state is characterized by increased chromatin accessibility at IRF loci, enhanced antiviral gene expression, and elevated interferon production in myeloid cells.

22. The method of claim 19 wherein epigenetic monitoring is performed with a single cell technique, optionally EpiTOF (Epigenetic landscape profiling using cytometry by Time-Of-Flight), single-cell ATAC-seq, or single-cell RNA-seq.

23-24. (canceled)

25. The method of claim 22, where EpiTOF, ATAC-seq, or single-cell ATAC-seq is used to determine the epigenetic state of innate cells, which is used as a biomarker to assess susceptibility or resistance to viral infection.

26. The method of claim 25, where an epigenetic signature in monocytes and mDCs or other cells, characterized by enhanced chromatin accessibility of the IRF1, IRF2, STAT1, STAT2, IRF7, IRF8, STAT5b, USF1 loci, is used as a biomarker to predict susceptibility or resistance to viral infection.

27. The method of claim 26, where an epigenetic signature in monocytes and mDCs or other cells, characterized by reduced chromatin accessibility of the AP-1, Jun, Fos loci, is used as a biomarker to predict susceptibility or enhanced inflammation or sepsis in viral or bacterial infections.
